SEQUENCE LISTING (89 sequences)

SEQ ID NO: 1 - DNA, 456 bp, Homo sapiens
tctttcgcacagtaatatacggccgtgtcctcggctctcaggctgttcatttgcagatacagcgtgttcttggaattgtctctggagatggtgaaccggcccttcacggagtctgcgtagtatgtgctaccaccactaccactaatagctgagacccactccagccccttccctggagcctggcggacccagctcatggcatagctgctaaaggtgaatccagaggctgcacaggagagtctcagggaccccccaggctgtaccaagcctcccccagactccaacagctgcacctcacactggacacctgcaaacaaaaagaaaccctggtcagaaactgccacacgtatccactgtttctctcactcttatccattcacactcaatttttctatttctccatgaattaccttttaaaatagccacaagaaaaagccagctcagcccaaactccat

SEQ ID NO: 2 - DNA, 296 bp, Homo sapiens
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaga

SEQ ID NO: 3 - protein, 98 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 4 - DNA, 481 bp, Homo sapiens
atgaccctgctgcaggtggatgggctcggcggggctgaaatccccccacacagtgctcatgtgctcacactgccttagggctctttcatccctggatctgtgtccaggccaggcacgtgggaagatttacttggagttcagctcctcagtttcaagccttttctctcccgttttctctcctgtaggatccgtggcctcctatgagctgactcagccaccctcagtgtccgtgtccccaggacagacagccagcatcacctgctctggagataaattgggggataaatatgcttgctggtatcagcagaagccaggccagtcccctgtgctggtcatctatcaagatagcaagcggccctcagggatccctgagcgattctctggctccaactctgggaacacagccactctgaccatcagcgggacccaggctatggatgaggctgactattactgtcaggcgtgggacagcagcactgca

SEQ ID NO: 5 - DNA, 285 bp, Homo sapiens
tcctatgagctgactcagccaccctcagtgtccgtgtccccaggacagacagccagcatcacctgctctggagataaattgggggataaatatgcttgctggtatcagcagaagccaggccagtcccctgtgctggtcatctatcaagatagcaagcggccctcagggatccctgagcgattctctggctccaactctgggaacacagccactctgaccatcagcgggacccaggctatggatgaggctgactattactgtcaggcgtgggacagcagcactgca

SEQ ID NO: 6 - protein, 95 aa, Homo sapiens
QSVLTQPPSVSVSPGQTASITCSGDKLGDKYACWYQQKPGQSPVLVIYQDSKRPSGIPERFSGSNSGNTATLTISGTQAMDEADYYCQAWDSSTA

SEQ ID NO: 7 - DNA, 781 bp, Homo sapiens
atggcctggaccgttctcctcctcggcctcctctctcactgcacaggtgatccccccagggtctcaccaacctgcccagcccaagggttctgggtccagcgtgtccttgattctgagctcaggagggcccttcctgtggtgggcaggatgctcatgaccctgctgcagggtgggaggctggtggggctgaactccccccaaactgtgctcaaaggcttgtgagagcctgagggactgcacctgccaggagagagtagtgagttttcagttcaaagtctccatacaacaggaaagtcatgggccactggggctggggctgattgcaggggataccctgagggttcacagactctctggagcttgtctgggacagcagggcaagggatttcataagaagcatctttcacctgcaagccaacctctctcttatttatttatttatttatttatttatttatttatttatttttatctttgcaggctctgtgacctcctatgtgctgactcagccaccctcggtgtcagtggccccaggacagacggccaggattacctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcatcc

SEQ ID NO: 8 - DNA, 290 bp, Homo sapiens
tcctatgtgctgactcagccaccctcggtgtcagtggccccaggacagacggccaggattacctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcatcc

SEQ ID NO: 9 - protein, 97 aa, Homo sapiens
QSVLTQPPSVSVAPGQTARITCGGNNIGSKSVHWYQQKPGQAPVLVVYDDSDRPSGIPERFSGSNSGNTATLTISRVEAGDEADYYCQVWDSSSDHP

SEQ ID NO: 10 - DNA, 478 bp, homo sapiens
atggcctgggctccactacttctcaccctcctcgctcactgcacaggtggctgcctgcaaggaattcagggagcgttcctggatgtcacctgggctgatgatctgttcctcctgcctgggaaccagtcttcatctctccccactgatctctgtgttgctctcttcttgcaggttcttgggccaattttatgctgactcagccccactctgtgtcggagtctccggggaagacggtaaccatctcctgcaccggcagcagtggcagcattgccagcaactatgtgcagtggtaccagcagcgcccgggcagtgcccccaccactgtgatctatgaggataaccaaagaccctctggggtccctgatcggttctctggctccatcgacagctcctccaactctgcctccctcaccatctctggactgaagactgaggacgaggctgactactactgtcagtcttatgatagcagcaatca

SEQ ID NO: 11 - DNA, 296 bp, Homo sapiens
aattttatgctgactcagccccactctgtgtcggagtctccggggaagacggtaaccatctcctgcaccggcagcagtggcagcattgccagcaactatgtgcagtggtaccagcagcgcccgggcagtgcccccaccactgtgatctatgaggataaccaaagaccctctggggtccctgatcggttctctggctccatcgacagctcctccaactctgcctccctcaccatctctggactgaagactgaggacgaggctgactactactgtcagtcttatgatagcagcaatca

SEQ ID NO: 12 - protein, 98 aa, Homo sapiens
NFMLTQPHSVSESPGKTVTISCTGSSGSIASNYVQWYQQRPGSAPTTVIYEDNQRPSGVPDRFSGSIDSSSNSASLTISGLKTEDEADYYCQSYDSSN

SEQ ID NO: 13 - DNA, 462 bp, homo sapiens
atgacctgctcccctctcctcctcacccttctcattcactgcacaggtgcccagacacagggtcaggggaggggtccaggaagcccatgaggccctgctttctccttctctctctagaccaagaatcaccgtgtctgtgtctctcctgcttccagggtcctgggcccagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatagcagcctgagtgctgg

SEQ ID NO: 14 - DNA, 296 bp, Homo sapiens
cagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatagcagcctgagtgctgg

SEQ ID NO: 15 - protein, 99 aa, Homo sapiens
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAG

SEQ ID NO: 16 - DNA, 466 bp, homo spaiens
atggcctggtctcctctcctcctcactctcctcgctcactgcacaggtgactggatacaggtccaggggaggggccctgggaagcctatggattcttgctttctcctgttgtctctagaagccgaataatgatgcctgtgtctctcccacttccagggtcctgggcccagtctgtgctgacgcagccgccctcagtgtctggggccccagggcagagggtcaccatctcctgcactgggagcagctccaacatcggggcaggttatgatgtacactggtaccagcagcttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacagcagcctgagtggttc

SEQ ID NO: 17 - DNA, 299 bp, Homo sapiens
cagtctgtgctgacgcagccgccctcagtgtctggggccccagggcagagggtcaccatctcctgcactgggagcagctccaacatcggggcaggttatgatgtacactggtaccagcagcttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacagcagcctgagtggttc

SEQ ID NO: 18 - protein, 100 aa, Homo sapiens
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGS

SEQ ID NO: 19 - DNA, 468 bp, homo sapiens
atggccagcttccctctcctcctcaccctcctcactcactgtgcaggtgacaggatggggaccaagaaaggggccctgggaagcccatggggccctgctttctcctcttgtctccttttgtctcttgtcaatcaccatgtctgtgtctctctcacttccagggtcctgggcccagtctgtgctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggtcc

SEQ ID NO: 20 - DNA, 296 bp, Homo sapiens
cagtctgtgctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggtcc

SEQ ID NO: 21 - protein, 99 aa, Homo sapiens
QSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGP

SEQ ID NO: 22 - DNA, 468 bp, homo sapiens
atggccggcttccctctcctcctcaccctcctcactcactgtgcaggtgacaggatggggaccaagagaggggccctgggaagcccatggggccctgctttctcctcttgtctcctttcgtctcttgtcaatcaccatgtctgtgtctctctcacttccagggtcctgggcccagtctgtgctgactcagccaccctcagcgtctgggacccccgggcagagggtcaccatctcttgttctggaagcagctccaacatcggaagtaattatgtatactggtaccagcagctcccaggaacggcccccaaactcctcatctatagtaataatcagcggccctcaggggtccctgaccgattctctggctccaagtctggcacctcagcctccctggccatcagtgggctccggtccgaggatgaggctgattattactgtgcagcatgggatgacagcctgagtggtcc

SEQ ID NO: 23 - DNA, 296 bp, Homo sapiens
cagtctgtgctgactcagccaccctcagcgtctgggacccccgggcagagggtcaccatctcttgttctggaagcagctccaacatcggaagtaattatgtatactggtaccagcagctcccaggaacggcccccaaactcctcatctatagtaataatcagcggccctcaggggtccctgaccgattctctggctccaagtctggcacctcagcctccctggccatcagtgggctccggtccgaggatgaggctgattattactgtgcagcatgggatgacagcctgagtggtcc

SEQ ID NO: 24 - protein, 99 aa, Homo sapiens
QSVLTQPPSASGTPGQRVTISCSGSSSNIGSNYVYWYQQLPGTAPKLLIYSNNQRPSGVPDRFSGSKSGTSASLAISGLRSEDEADYYCAAWDDSLSGP

SEQ ID NO: 25 - DNA, 493 bp, homo sapiens
atggcctggacccctctctggctcactctcctcactctttgcataggtgctgcctcccagggctcaaccccatattatcatgctagctgtgccaacctggccctgagcttcggctcaacacagggagtagtgtagggtgtgggactctaggcgtgaaacccttatcctcacctcttctgtcctcttttgcaggttctgtggtttcttctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaaccatct

SEQ ID NO: 26 - DNA, 290 bp, Homo sapiens
tcttctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaaccatct

SEQ ID NO: 27 - protein, 97 aa, Homo sapiens
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHL

SEQ ID NO: 28 - protein, 8 aa, Artificial Sequence
  CDR Variant Sequence 1
XXXGGXXX

SEQ ID NO: 29 - protein, 7 aa, Artificial Sequence
  CDR Variant Sequence
XXXGXXX

SEQ ID NO: 30 - protein, 11 aa, Homo sapiens
VFGTGTKVTVS

SEQ ID NO: 31 - protein, 11 aa, Homo sapiens
VFGGGTKLTVS

SEQ ID NO: 32 - protein, 11 aa, Homo sapiens
VFGGGTKLTVS

SEQ ID NO: 33 - protein, 11 aa, Homo sapiens
VFGGGTQLIIS

SEQ ID NO: 34 - protein, 11 aa, Homo sapiens
VFGEGTELTVS

SEQ ID NO: 35 - protein, 11 aa, Homo sapiens
VFGSGTKVTVS

SEQ ID NO: 36 - protein, 11 aa, Homo sapiens
VFGGGTQLTAS

SEQ ID NO: 37 - protein, 11 aa, Homo sapiens
VFGTGTKLIIS

SEQ ID NO: 38 - DNA, 105 bp, Artificial Sequence
  PCR primer
gatcagggtctgagacgagaccgtcactttcgtaccggtgccgaacaccacagtannannanntccgccannannanngtcccacgcctgacagtaatagtcagc

SEQ ID NO: 39 - protein, 31 aa, Homo sapiens
  VARIANT (10)..(12): X is any amino acid other than Trp, Gln, Lys, Glu, Met
ADYYCQAWDXXXGGXXXTVVFGTGTKVTVSS

SEQ ID NO: 40 - DNA, 105 bp, Homo sapiens
  misc_feature (65)..(66): n is a, c, g, or t
gatcagggtctgagacccgctgctcacggtaaccatggtaccttgaccccaaatatcaaacgcannannanngccannannanntttcgcacagtagtaaacagc

SEQ ID NO: 41 - protein, 25 aa, Homo sapiens
  VARIANT (7)..(9): X is any amino acid other than Trp, Gln, Lys, Glu, Met
VYYCAKXXXGXXXAFDIWGQGTMVT

SEQ ID NO: 42 - DNA, 771 bp, Homo sapiens
  misc_feature (286)..(287): n is a, c, g, or t
atgggagacggtcagtctgtgctgactcagccaccctcagtgtccgtgtccccaggacagacagccagcatcacctgctctggagataaattgggggataaatatgcttgctggtatcagcagaagccaggccagtcccctgtgctggtcatctatcaagatagcaagcggccctcagggatccctgagcgattctctggctccaactctgggaacacagccactctgaccatcagcgggacccaggctatggatgaggctgactattactgtcaggcgtgggacnntnntnntggaggtnntnntnntactgtggtgttcggcacgggcaccaagctcatcatttcgtctcagaccggtggttctggtggtggtggttctggcggcggcggctccggtggtggtggatccgaagtccaactgctggagtccggcggtggcctggtgcagccaggtggcagcctgcgcctgagctgcgccgcatccggttttactttcagcagctacgcgatgtcgtgggtgcgccaggcaccgggcaagggcctggagtgggtcagcgccatcagcggtagcggcggttctacgtattatgcggacagcgtcaagggccgtttcaccatcagccgtgacaattccaaaaacaccctgtacttgcagatgaacagcttgcgtgcggaagatacggctgtttactactgtgcgaaanntnntnntgganntnntnntgcctttgatatttggggtcaaggtaccatggttaccgtgagcagc

SEQ ID NO: 43 - protein, 257 aa, Homo sapiens
  misc_feature (96)..(98): Xaa can be any naturally occurring amino acid
MGDGQSVLTQPPSVSVSPGQTASITCSGDKLGDKYACWYQQKPGQSPVLVIYQDSKRPSGIPERFSGSNSGNTATLTISGTQAMDEADYYCQAWDXXXGGXXXTVVFGTGTKVTVSSQTGGSGGGGSGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKXXXGXXXAFDIWGQGTMVTVSS

SEQ ID NO: 44 - DNA, 361 bp, Homo sapiens
atgggagacggtcagtctgtgctgactcagccaccctcagtgtccgtgtccccaggacagacagccagcatcacctgctctggagataaattgggggataaatatgcttgctggtatcagcagaagccaggccagtcccctgtgctggtcatctatcaagatagcaagcggccctcagggatccctgagcgattctctggctccaactctgggaacacagccactctgaccatcagcgggacccaggctatggatgaggctgactattactgtcaggcgtgggactgagacctagacggtctctgcgtttgatatttggggtcaaggtaccatggttaccgtgagcagctcgtctcagacc

SEQ ID NO: 45 - DNA, 396 bp, Homo sapiens
actgtggtgttcggcaccggtacgaaagtgactgtctcatctcagaccggtggttctggtggtggtggttctggcggcggcggctccggtggtggtggatccgaagtccaactgctggagtccggcggtggcctggtgcagccaggtggcagcctgcgcctgagctgcgccgcatccggttttactttcagcagctacgcgatgtcgtgggtgcgccaggcaccgggcaagggcctggagtgggtcagcgccatcagcggtagcggcggttctacgtattatgcggacagcgtcaagggccgtttcaccatcagccgtgacaattccaaaaacaccctgtacttgcagatgaacagcttgcgtgcggaagatacggctgtttactactgtgcgaaa

SEQ ID NO: 46 - protein, 8 aa, Homo sapiens
PFGGGGYV

SEQ ID NO: 47 - protein, 7 aa, Homo sapiens
PPHGAPA

SEQ ID NO: 48 - protein, 8 aa, Homo sapiens
LCIGGVAS

SEQ ID NO: 49 - protein, 7 aa, Homo sapiens
HNSGNNF

SEQ ID NO: 50 - protein, 8 aa, Homo sapiens
FVSGGIST

SEQ ID NO: 51 - protein, 7 aa, Homo sapiens
FNFGNAY

SEQ ID NO: 52 - protein, 8 aa, Homo sapiens
INSGGASF

SEQ ID NO: 53 - protein, 7 aa, Homo sapiens
  misc_feature (1)..(3): Xaa can be any naturally occurring amino acid
XXXGTNY

SEQ ID NO: 54 - protein, 8 aa, Homo sapiens
SRAGGCNG

SEQ ID NO: 55 - protein, 7 aa, Homo sapiens
FDYGHCI

SEQ ID NO: 56 - protein, 8 aa, Homo sapiens
TNRGGVCA

SEQ ID NO: 57 - protein, 7 aa, Homo sapiens
TAAGVPF

SEQ ID NO: 58 - protein, 8 aa, Homo sapiens
FSTGGCAF

SEQ ID NO: 59 - protein, 7 aa, Homo sapiens
AICGATA

SEQ ID NO: 60 - protein, 8 aa, Homo sapiens
  misc_feature (2)..(2): Xaa can be any naturally occurring amino acid
FXGGGDGT

SEQ ID NO: 61 - protein, 7 aa, Homo sapiens
PYRGSFF

SEQ ID NO: 62 - protein, 8 aa, Homo sapiens
IIPGGLYA

SEQ ID NO: 63 - protein, 7 aa, Homo sapiens
PVIGSNT

SEQ ID NO: 64 - protein, 266 aa, Homo sapiens
MGDGQSVLTQPPSVSVSPGQTASITCSGDKLGDKYACWYQQKPGQSPVLVIYQDSKRPSGIPERFSGSNSGNTATLTISGTQAMDEADYYCQAWDFNLGGCGDTVVFGTGTKVTVSSQTGGSGGGGSGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKHIDGPVAAFDIWGQGTMVTVSSSSQTSILVA

SEQ ID NO: 65 - protein, 89 aa, Homo sapiens
QSVLTQPPSVSVSPGQTASITCSGDKLGDKYACWYQQKPGQSPVLVIYQDSKRPSGIPERFSGSNSGNTATLTISGTQAMDEADYYCQA

SEQ ID NO: 66 - protein, 93 aa, Homo sapiens
MGDGQSVLTQPPSVSVSPGQTASITCSGDKLGDKYACWYQQKPGQSPVLVIYQDSKRPSGIPERFSGSNSGNTATLTISGTQAMDEADYYCQA

SEQ ID NO: 67 - protein, 93 aa, Homo sapiens
MGDGQSVLTQPPSVSVSPGQTASITCSGDKLGDKYACWYQQKPGQSPVLVIYQDSKRPSGIPERFSGSNSGNTATLTISGTQAMDEAHYYCQT

SEQ ID NO: 68 - protein, 93 aa, Homo sapiens
MGDGQSVLTQPPSVSVSPGQTATITCSGDKLGDKFACWYQQKPGQSPVLVIYQDYQRPSGIPERFSGSNSGNTATLTISGTQVMDEADYYCQA

SEQ ID NO: 69 - protein, 87 aa, Homo sapiens
  misc_feature (41)..(41): Xaa can be any naturally occurring amino acid
MGDGQSVLTQPPAVSVAPEKTATIACGGNRIGSKSVHWYQXKPGQAPVLVIYNDNDRPSGXPERFSGSNSGNTATLTISRVEVGDEA

SEQ ID NO: 70 - protein, 87 aa, Homo sapiens
MGDGQSVLTQPPSVSVAPGKTATIACGGNRIGSKSVHWYQQKPGQAPVLVIYNDNDRPSGIPERFSGSNSGNTATLTISRVEVGDEA

SEQ ID NO: 71 - protein, 83 aa, Homo sapiens
QSVLTQPPSVSVAPGKTARITCGGNNIGSKSVHWYQQKPGQAPVLVIYYDSDRPSGIPERFSGSNSGNTATLTISRVEAGDEA

SEQ ID NO: 72 - protein, 87 aa, Homo sapiens
MGDGQSVLTQPPSVSVAPGKTATITCGGNNIEDKSVHWYQQKPGQAPVLVIYYDTDRPSGIPERFSGSNSGNTATLTINRVEAGDEA

SEQ ID NO: 73 - protein, 95 aa, Homo sapiens
NFMLTQPHSVSESPGKTVTISCTGSSGSIASNYVQWYQQRPGSAPTTVIYEDNQRPSGVPDRFSGSIDSSSNSASLTISGLKTEDEADYYCQSYD

SEQ ID NO: 74 - protein, 99 aa, Homo sapiens
MGDGNFMLTQPHSVSESPGKTVTIPCTGSSGSIASNYVQWYQQRPGSAPTTVIYEDKQKPSGVPDRFSGSIDSSSNSASLTISGLKTEDEADYYCQSYD

SEQ ID NO: 75 - protein, 99 aa, Homo sapiens
MGDGNFMLTQPHSVSESPGKTVTISCTGSSGSIASNSVQRYQQRPGSAPTTVIYEDNRRPSGVPDRFSGSIDSSSNSASLIISGLQTEDEADYYCQSYD

SEQ ID NO: 76 - protein, 99 aa, Homo sapiens
MGDGNFMLTQPHSVSESPGKTVTISCTGRSGSIADNYVQWYQQRPGSVPTSVIFEDNQRPSGVPERFSGSIDSSSNSASLTISGLMTEDEADYHCQSYD

SEQ ID NO: 77 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 78 - protein, 100 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKKVEP

SEQ ID NO: 79 - DNA, 1403 bp, Artificial Sequence
  Fab polynucleotide sequence
atgggagacggtcagagcgtgttgacgcagccaccgtcggttagcgtgtctccgggccaaactgcttccatcacctgtagcggcgataagctgggtgacaagtatgcgtgttggtatcagcagaaaccgggtcaaagcccggttctggtcatctaccaagatagcaagcgtccgtcgggtatcccggagcgttttagcggcagcaattccggtaatacggcgaccctgaccatctccggcacgcaggctatggatgaggcagactactattgccaggcgtgggatnnnnnnggtnnnnnnaccgtggtctttggcacgggtaccaaagttaccgttctgggccagccgaaagcagcgccgagcgtgacgctgttcccgccgtccagcgaagaactgcaagcgaacaaagcgacgctggtgtgcctgattagcgacttttatccaggtgccgtcaccgtcgcgtggaaagcggacagctccccagtcaaagcgggcgttgaaaccacgaccccgagcaagcagtctaacaacaaatacgcggcctctagctacttgtccctgaccccggaacaatggaagagccaccgcagctacagctgtcaggtcacccatgagggtagcaccgtggaaaagacggtggcacctaccgagtgtagcgattacaaggacgacgacgataaggagaatacctgatgggtagcgaggtgcagctgctggaatccggtggtggcttggtgcaaccgggtggttcgttgcgtctgagctgtgccgccagcggtttcacctttagcagctatgcaatgtcctgggtgcgccaagcgcctggtaagggcttggagtgggtgtctgcaattagcggtagcggcggtagcacctattacgccgatagcgtcaagggtcgcttcactattagccgtgataacagcaaaaacacgctgtatctgcaaatgaattccctgcgtgcagaggacacggcggtctactactgcgcaaaannnnnnnnnggtnnnnnnnnngcattcgacatttggggccagggtaccatggttaccgttagcagcgccagcacgaagggcccgagcgttttcccgctggcgccgtgcagccgtagcacgtctggcggtacggctgctctgggctgcctggttaaggactatttcccggagccggttaccgtgagctggaactctggtgcgctgactagcggtgtccatacctttccggccgtgctgcagagcagcggtctgtacagcctgtctagcgttgtcaccgttcctagctctagcctgggcactcagacgtacacctgcaatgttaatcacaaaccgagcaataccaaggtcgataagaaagttgagccgaaaagctgttcgtctcagacccaccaccatcatcaccactaa

SEQ ID NO: 80 - protein, 466 aa, Artificial Sequence
  Amino acid sequence of Fab
MGDGQSVLTQPPSVSVSPGQTASITCSGDKLGDKYACWYQQKPGQSPVLVIYQDSKRPSGIPERFSGSNSGNTATLTISGTQAMDEADYYCQAWDXXGXXTVVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSDYKDDDDKENTMGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKXXXGXXXAFDIWGQGTMVTVSSASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKKVEPKSCSSQTHHHHHH

SEQ ID NO: 81 - protein, 233 aa, Artificial Sequence
  Fab
MGDGQSVLTQPPSVSVSPGQTASITCSGDKLGDKYACWYQQKPGQSPVLVIYQDSKRPSGIPERFSGSNSGNTATLTISGTQAMDEADYYCQAWDFNLGGCGDTVVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSDYKDDDDKENT

SEQ ID NO: 82 - protein, 236 aa, Artificial Sequence
  VH domain linked to IGHG3 CH1
MGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKHIDGPVAAFDIWGQGTMVTVSSASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKKVEPKSCSSQTHHHHHH

SEQ ID NO: 83 - DNA, 1412 bp, Artificial Sequence
  anti-mAG1 Fab polynucleotide sequence
atgggagacggtcagagcgtgttgacgcagccaccgtcggttagcgtgtctccgggccaaactgcttccatcacctgtagcggcgataagctgggtgacaagtatgcgtgttggtatcagcagaaaccgggtcaaagcccggttctggtcatctaccaagatagcaagcgtccgtcgggtatcccggagcgttttagcggcagcaattccggtaatacggcgaccctgaccatctccggcacgcaggctatggatgaggcagactactattgccaggcgtgggatttcaacctgggtggttgcggtgataccgtggtctttggcacgggtaccaaagttaccgttctgggccagccgaaagcagcgccgagcgtgacgctgttcccgccgtccagcgaagaactgcaagcgaacaaagcgacgctggtgtgcctgattagcgacttttatccaggtgccgtcaccgtcgcgtggaaagcggacagctccccagtcaaagcgggcgttgaaaccacgaccccgagcaagcagtctaacaacaaatacgcggcctctagctacttgtccctgaccccggaacaatggaagagccaccgcagctacagctgtcaggtcacccatgagggtagcaccgtggaaaagacggtggcacctaccgagtgtagcgattacaaggacgacgacgataaggagaatacctgatgggtagcgaggtgcagctgctggaatccggtggtggcttggtgcaaccgggtggttcgttgcgtctgagctgtgccgccagcggtttcacctttagcagctatgcaatgtcctgggtgcgccaagcgcctggtaagggcttggagtgggtgtctgcaattagcggtagcggcggtagcacctattacgccgatagcgtcaagggtcgcttcactattagccgtgataacagcaaaaacacgctgtatctgcaaatgaattccctgcgtgcagaggacacggcggtctactactgcgcaaaacacatcgatggtccggtggcggcattcgacatttggggccagggtaccatggttaccgttagcagcgccagcacgaagggcccgagcgttttcccgctggcgccgtgcagccgtagcacgtctggcggtacggctgctctgggctgcctggttaaggactatttcccggagccggttaccgtgagctggaactctggtgcgctgactagcggtgtccatacctttccggccgtgctgcagagcagcggtctgtacagcctgtctagcgttgtcaccgttcctagctctagcctgggcactcagacgtacacctgcaatgttaatcacaaaccgagcaataccaaggtcgataagaaagttgagccgaaaagctgttcgtctcagacccaccaccatcatcaccactaa

SEQ ID NO: 84 - protein, 919 aa, Artificial Sequence
  anti-mAG1 Fab::PG::SNAP::DBP fusion
MGDGQSVLTQPPSVSVSPGQTASITCSGDKLGDKYACWYQQKPGQSPVLVIYQDSKRPSGIPERFSGSNSGNTATLTISGTQAMDEADYYCQAWDFNLGGCGDTVVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSDYKDDDDKENTMGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKHIDGPVAAFDIWGQGTMVTVSSASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKKVEPKSCSSQTSLIEVEKPLYGVEVFVGETAHFEIELSEPDVHGQWKLKGQPLTASPDCEIIEDGKKHILILHNCQLGMTGEVSFQAANAKSAANLKVKELNSSSQTSGTRHHNSHNSSGTNSPPKVLRKGDRGDEVCQLQTLLNLCGYDVGKPDGIFGNNTFNQVVKFQKDNCLDSDGIVGKNTWAELFSKYSPPSMDKDCEMKRTTLDSPLGKLELSGCEQGLHEIKLLGKGTSAADAVEVPAPAAVLGGPEPLMQATAWLNAYFHQPEAIEEFPVPALHHPVFQQESFTRQVLWKLLKVVKFGEVISYQQLAALAGNPAATAAVKTALSGNPVPILIPCHRVVSSSGAVGGYEGGLAVKEWLLAHEGHRLGKPGLGPAGNSGKGAVNINAASQQELEALPGIGPAKAKAIAEYRAQNGAFKSVDDLIKVKGIGPAVLAKLKDQASVGAPAPKGPAKPVLPAVKK

SEQ ID NO: 85 - protein, 8 aa, Artificial Sequence
  FLAG epitope
DYKDDDDK

SEQ ID NO: 86 - protein, 20 aa, Artificial Sequence
  VL CDR3 + J region sequence
SSRNTVVFGTGTKVTVSSQT

SEQ ID NO: 87 - DNA, 1892 bp, Artificial Sequence
  IGLV6-57/IGHV3-23::CaM Fab sequence
tctagaatgggagacggtaattttatgctgactcagccccactctgtgtcggagtctccggggaagacggtaaccatctcctgcacccgcagcagtggcagcattgccagcaactatgtgcagtggtaccagcagcgcccgggcagtgcccccaccactgtgatctatgaggataaccaaagaccctctggggtccctgatcggttctctggctccatcgacagctcctccaactctgcctccctcaccatctctggactgaagactgaggacgaggctgactactactgtcagtcttatgattcatcgcgcaacaccgtggtctttggcacgggtaccaaagttaccgttctgggccagccgaaagcagcgccgagcgtgacgctgttcccgccgtccagcgaagaactgcaagcgaacaaagcgacgctggtgtgcctgattagcgacttttatccaggtgccgtcaccgtcgcgtggaaagcggacagctccccagtcaaagcgggcgttgaaaccacgaccccgagcaagcagtctaacaacaaatacgcggcctctagctacttgtccctgaccccggaacaatggaagagccaccgcagctacagctgtcaggtcacccatgagggtagcaccgtggaaaagacggtggcacctaccgagtgtagcgattacaaggacgacgacgataaggagaatacctgatgggtagcgaggtgcagctgctggaatccggtggtggcttggtgcaaccgggtggttcgttgcgtctgagctgtgccgccagcggtttcacctttagcagctatgcaatgtcctgggtgcgccaagcgcctggtaagggcttggagtgggtgtctgcaattagcggtagcggcggtagcacctattacgccgatagcgtcaagggtcgcttcactattagccgtgataacagcaaaaacacgctgtatctgcaaatgaattccctgcgtgcagaggacacggcggtctactactgcgcaaaacacatcgatggtccggtggcggcattcgacatttggggccagggtaccatggttaccgttagcagcgccagcacgaagggcccgagcgttttcccgctggcgccgtgcagccgtagcacgtctggcggtacggctgctctgggctgcctggttaaggactatttcccggagccggttaccgtgagctggaactctggtgcgctgactagcggtgtccatacctttccggccgtgctgcagagcagcggtctgtacagcctgtctagcgttgtcaccgttcctagctctagcctgggcactcagacgtacacctgcaatgttaatcacaaaccgagcaataccaaggtcgataagaaagttgagccgaaaagctgttcgagctcgtctcagaccagcgcggaccaactgaccgaagaacaaatcgcggagttcaaagaagcgttctccctgtttgacaaagatggcgatggtacgattaccaccaaagaactgggcaccgttatgcgcagcctgggccagaatccgacggaggcggagctgcaggacatgattaacgaggttgacgcggatggtaacggcaccattgactttccggagtttttgacgatgatggcacgtaagatgaaggatactgatagcgaagaagagatccgtgaggcatttcgtgtgttcgataaggatggtaatggttacatcagcgccgcagaactgcgccacgtgatgacgaatctgggtgagaagctgaccgacgaagaggtcgatgagatgattcgtgaggccgacatcgacggtgacggtcaggtcaactatgaagagttcgttcaaatgatgaccgctggttccaacagtgattacaaggacgatgatgacaaataagaattc

SEQ ID NO: 88 - protein, 392 aa, Artificial Sequence
  Fab heavy chain::Calmodulin fusion
MGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKHIDGPVAAFDIWGQGTMVTVSSASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKKVEPKSCSSSSQTSADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGSNSDYKDDDDK

SEQ ID NO: 89 - protein, 149 aa, Artificial Sequence
  gpD::CBP fusion
MTSKETFTHYQPQGNSDPAHTATAPGGLSAKAPAMTPLMLDTSSRKLVAWDGTTDGAAVGILAVAADQTSTTLTFYKSGTFRYEDVLWPEAASDETKKRTAFAGTAISIVGGSGGSSNDGTNSSRRWKKAFIAVSAANRFKKISSSNSS

(19) United States
(12) Patent Application Publication
(10) Pub. No.: US 2016/0002317 A1
(43) Pub. Date: Jan. 7, 2016

(54) Cytoplasmic Expression of Fab Proteins

(71) Applicant: AFFINITY BIOSCIENCES PTY LTD, Scoresby, Victoria (AU)

(72) Inventors: Matthew Beasley, Fitzroy North, Victoria (AU); Benjamin Kiefel, Vermont South, Victoria (AU); Keith Niven, Forest Hill, Victoria (AU)

(73) Assignee: Affinity Biosciences of Fab Proteins, Scoresby, Victoria (AU)

(21) Appl. No.: 14/768,678

(22) PCT Filed: Feb. 20, 2014
(86) PCT No.: PCT/IB2014/059107

§ 371 Date: Aug. 18, 2015

(30) Foreign Application Priority Data
Feb. 20, 2013 (AU) 2013900572

(51) Int. Cl. C07K 16/00; C12N 15/10

(52) U.S. Cl. CPC C07K 16/005; C12N 15/1093; C07K 2317/31; C07K 2317/55; C07K 2319/00; C07K 2317/94

(57) ABSTRACT

The present invention relates generally to antigen binding polypeptides, such as Fab fragments and derivatives thereof, that demonstrate high stability and solubility. The present invention also relates to polynucleotides encoding such polypeptides, to libraries of such polypeptides or polynucleotides, and to methods of using such polypeptides in research, diagnostic and therapeutic applications. For example, the polypeptides can be used in screening methods to identify a polypeptide that binds to a particular target molecule.

### FIELD OF THE INVENTION

The present invention relates generally to antigen binding polypeptides, such as Fab fragments and derivatives thereof, that demonstrate high stability and solubility. In particular, the present invention relates to Fab fragments and derivatives thereof comprising paired V_{L} and V_{H} domains that demonstrate soluble expression and folding in a reducing or intracellular environment. The present invention also relates to polynucleotides encoding such polypeptides, to libraries of such polypeptides or polynucleotides, and to methods of using such polypeptides in research, diagnostic and therapeutic applications. For example, the polypeptides can be used in screening methods to identify a polypeptide that binds to a particular target molecule.

### BACKGROUND OF THE INVENTION

The vertebrate antibody repertoire was formed by the duplication and diversification of ancestral genes of a heterodimer of two immunoglobulin (Ig) folds.

The diversity generated by the immune system relies not only on the germline gene families of Ig genes, but from the recombination of subdomain exons in vivo during B- and T-cell development to form numerous unique lineages with additional diversity at the exon boundaries that occur at surface-exposed loops of the Ig protein. This process of recombination is called V(D)J recombination, so called after the two variable light (V_{L}) and three variable heavy (V_{H}) exons that recombine to form the N-terminal antigen binding domains of the light chain and heavy chain of the antibody, respectively. However, as the duplicated genes diverged from their ancestral pair, the cumulated effect of mutations has resulted in a less-than-perfect interfacial fit between heterodimer units of the variable domains. Selection pressure is not applied to any one gene, but to the family as a whole. Thus, maximum diversity, which is a good thing for the immune system, can result in less-than-ideal folding stabilities for individual family members. Furthermore, the binding domains themselves may have different folding stabilities. The requirement to form a functional heterodimer from numerous diverged subunits is compensated for by the presence of conserved disulphide bonds between the β-sheets of the domains. However, the interface may still not be a stable fit, requiring a folding checkpoint in the ER.

As a result of the ‘consensus’ approach to a protein fit applied by the antibody variable domains, some pairings have a low folding stability and propensity for either poor expression in bacterial/mammalian hosts, and a propensity to aggregate. Furthermore, in almost all cases, there is a total requirement for the inter-sheet disulphide bonds to be formed within the V_{L} and V_{H} domains. This necessitates that for expression of antibody libraries in a bacterial host such as E. coli the antibody is expressed in the periplasm of the cell, an oxidizing space that has disulphide chaperones and isomerases, and often as a fusion between the V_{L} and V_{H} domains (single chain antibody; scFv). However, export to the periplasm requires the extrusion through the inner membrane, which is saturated at the levels desired for high expression of the antibody, resulting in far lower yields than cytoplasmic expression.

Although there are reports demonstrating intracellular antibodies that have been productively folded in the reducing environment of the cytoplasm without disulphide bonds, these reports have always been of a minimal antibody region, either the V_{L} and V_{H} domains heterodimerised and connected by a peptide linker (scFv), or a single domain antibody (V_{H}H).

Once either a scFv or VHH antibody with affinity to a target has been isolated it may be desirable to convert it back to a larger antibody format, such as a Fab (Fragment, antigen binding) or mAb (monoclonal Antibody). However, it is not uncommon to reduce affinity to the target with such a conversion, presumably due to conformational changes in the relative orientation of the V domains as they are reattached to constant domains.

While scFvs have a much cheaper cost of production, and face possible reduction in affinity on conversion to Fab or mAbs, the need for an extended serum half-life or the presence of effector domains may demand their production as Fab or mAbs. As the Fab region of the antibody is essentially a monovalent form of the larger bivalent mAb, if a Fab is used as a library scaffold for antibody diversity, rather than a scFv, it is more likely to retain binding affinity on mAb conversion. Furthermore, it is useful in its own right as a therapeutic as the immune effector domains of a mAb may not be desired for target binding in vivo. Additionally, two Fab regions may be linked as F(ab′)₂ structures, in which a flexible hinge region links two arm domains. If the Fab arms have affinity to different targets they may thereby form bispecific antibodies, capable of binding two targets simultaneously.

There is therefore a need for a cost-effective, high yield and homogenous method of Fab production. Ideally, this would be in E. coli as it is also the host used for expression of many of the antibody libraries. The production of Fab proteins in a soluble form in the E. coli cytoplasm enables a much lower cost of production, as well as enabling cytoplasmic screening technologies.

### SUMMARY OF THE INVENTION

The present inventors have now developed methods of making polypeptide libraries comprising certain antibody heavy and light chains that are particularly effective at producing soluble and functional Fab forms of antibodies in the reducing environment of the cytoplasm and in high yield. Furthermore, the Fab heterodimers produced by the present inventors retain the binding specificities of their progenitors, such as scFv.

Accordingly, in one aspect, the invention provides a Fab library comprising a plurality of different Fab fragments or derivatives thereof, which comprise:

i) an antibody heavy chain variable region (V_{H}) comprising a scaffold region which is at least 90% identical to the scaffold region of IGHV3-23 as set out in SEQ ID NO: 3; and

ii) an antibody light chain variable region (V_{L}) comprising a scaffold region which is at least 90% identical to the scaffold region of any one of IGLV1-40 (as set out in SEQ ID NO: 18), IGLV1-44 (as set out in SEQ ID NO: 21), IGLV1-47 (as set out in SEQ ID NO: 24), IGLV1-51 (as set out in SEQ ID NO: 15), IGLV3-1 (as set out in SEQ ID NO: 6), IGLV3-19 (as set out in SEQ ID NO: 27), IGLV3-21 (as set out in SEQ ID NO: 9), IGLV6-57 (as set out in SEQ ID NO: 12);

wherein the V_{H} and the V_{L} are capable of forming an antigen-binding site; and wherein at least two of the Fab fragments or derivatives thereof differ from one another in the sequence of amino acids present in one or more complementarity determining regions (CDRs) in the V_{H} and/or V_{L} variable regions.

Preferably, the sequence of amino acids in one or more of the CDRs of the V_{H} and/or V_{L} variable domains is random or semi-random or is derived from a human antibody.

In one embodiment of the Fab library of the invention, the V_{L} comprises a scaffold region which is at least 90% identical to the scaffold region of IGLV3-1 as set out in SEQ ID NO 6.

In another aspect, the invention provides a method of constructing a Fab library, the method comprising preparing a plurality of different Fab fragments or derivatives thereof, which comprise:

i) an antibody heavy chain variable region (V_{H}) comprising a scaffold region which is at least 90% identical to the scaffold region of IGHV3-23 as set out in SEQ ID NO: 3; and

ii) an antibody light chain variable region (V_{L}) comprising a scaffold region which is at least 90% identical to the scaffold region of any one of IGLV1-40 (as set out in SEQ ID NO: 18), IGLV1-44 (as set out in SEQ ID NO: 21), IGLV1-47 (as set out in SEQ ID NO: 24), IGLV1-51 (as set out in SEQ ID NO: 15), IGLV3-1 (as set out in SEQ ID NO: 6), IGLV3-19 (as set out in SEQ ID NO: 27), IGLV3-21 (as set out in SEQ ID NO: 9), IGLV6-57 (as set out in SEQ ID NO: 12); wherein the V_{H} and the V_{L} are capable of forming an antigen-binding site; and wherein at least two of the Fab fragments or derivatives thereof differ from one another in the sequence of amino acids present in one or more CDRs in the V_{H} and/or V_{L} variable regions.

In another aspect, the invention provides a polynucleotide library comprising a plurality of different polynucleotides, wherein each polynucleotide encodes a Fab fragment or derivative thereof comprising:

i) an antibody heavy chain variable region (V_{H}) comprising a scaffold region which is at least 90% identical to the scaffold region of IGHV3-23 as set out in SEQ ID NO: 3; and

ii) an antibody light chain variable region (V_{L}) comprising a scaffold region which is at least 90% identical to the scaffold region of any one of IGLV1-40 (as set out in SEQ ID NO: 18), IGLV1-44 (as set out in SEQ ID NO: 21), IGLV1-47 (as set out in SEQ ID NO: 24), IGLV1-51 (as set out in SEQ ID NO: 15), IGLV3-1 (as set out in SEQ ID NO: 6), IGLV3-19 (as set out in SEQ ID NO: 27), IGLV3-21 (as set out in SEQ ID NO: 9), IGLV6-57 (as set out in SEQ ID NO: 12);

wherein the V_{H} and the V_{L} are capable of forming an antigen-binding site; and wherein at least two of the polynucleotides differ from one another by encoding Fab fragments or derivatives thereof comprising one or more different CDRs in the V_{H} and/or V_{L} variable regions.

Preferably, the polynucleotides encode a sequence of amino acids in one or more of the CDRs of the V_{H} and/or V_{L} variable domains that is random or semi-random or is derived from a human antibody.

In another aspect, the invention provides a method of constructing a polynucleotide library, the method comprising preparing a plurality of different polynucleotides encoding a Fab fragment or derivative thereof, which comprises:

i) an antibody heavy chain variable region (V_{H}) comprising a scaffold region which is at least 90% identical to the scaffold region of IGHV3-23 as set out in SEQ ID NO: 3; and

ii) an antibody light chain variable region (V_{L}) comprising a scaffold region which is at least 90% identical to the scaffold region of any one of IGLV1-40 (as set out in SEQ ID NO: 18), IGLV1-44 (as set out in SEQ ID NO: 21), IGLV1-47 (as set out in SEQ ID NO: 24), IGLV1-51 (as set out in SEQ ID NO: 15), IGLV3-1 (as set out in SEQ ID NO: 6), IGLV3-19 (as set out in SEQ ID NO: 27), IGLV3-21 (as set out in SEQ ID NO: 9), IGLV6-57 (as set out in SEQ ID NO: 12);

wherein the V_{H} and the V_{L} are capable of forming an antigen-binding site; and wherein at least two of the polynucleotides differ from one another by encoding Fab fragments or derivatives thereof comprising one or more different CDRs in the V_{H} and/or V_{L} variable regions.

In another aspect, the invention provides an isolated and/or recombinant Fab fragment or derivative thereof comprising:

i) an antibody heavy chain variable region (V_{H}) comprising a scaffold region which is at least 90% identical to the scaffold region of IGHV3-23 as set out in SEQ ID NO: 3; and

ii) an antibody light chain variable region (V_{L}) comprising a scaffold region which is at least 90% identical to the scaffold region of any one of IGLV1-40 (as set out in SEQ ID NO: 18), IGLV1-44 (as set out in SEQ ID NO: 21), IGLV1-47 (as set out in SEQ ID NO: 24), IGLV1-51 (as set out in SEQ ID NO: 15), IGLV3-1 (as set out in SEQ ID NO: 6), IGLV3-19 (as set out in SEQ ID NO: 27), IGLV3-21 (as set out in SEQ ID NO: 9), IGLV6-57 (as set out in SEQ ID NO: 12);

wherein the V_{H} and the V_{L} are capable of forming an antigen-binding site.

In one embodiment, the V_{L} preferably comprises a scaffold region which is at least 90% identical to the scaffold region of IGLV3-1 as set out in SEQ ID NO 6.

In another embodiment, the Fab fragment or derivative thereof is a Fab′ fragment, a F(ab′) fragment. In yet another embodiment, the Fab fragment or derivative thereof is bispecific or multispecific.

In one embodiment, the Fab fragment or derivative thereof is a fusion polypeptide. In one particular embodiment, the Fab fragment or derivative thereof is a fusion to calmodulin.

Preferably, the scaffold region of the V_{H} and/or V_{L} variable regions in the Fab fragment or the derivative thereof of the invention is at least 95%, 96%, 97%, 98% or 99% identical to the scaffold region of any of the given sequences.

The Fab fragment or derivative thereof of the invention is preferably soluble under reducing conditions. In addition, the Fab fragment or derivative thereof of the invention is preferably soluble and capable of stably forming an antigen-binding site when produced under reducing conditions.

In one embodiment of the preceding aspects of the invention, the Fab fragments or derivatives thereof can be retained in a soluble fraction of a cell lysate at a level of at least 25%. In another embodiment, the Fab fragments or derivatives thereof can be retained in a soluble fraction of a cell lysate at a level of at least 50%, or at least 75%.

In another preferred embodiment, the Fab fragment or derivative thereof of the invention is conjugated to a compound. The compound may be selected from the group consisting of a radioisotope, a detectable label, a therapeutic compound, a colloid, a toxin, a nucleic acid, a peptide, a protein, a compound that increases the half life of the polypeptide in a subject, and mixtures thereof.

In yet another embodiment, the Fab fragment or derivative thereof is a fusion protein that is fused to at least one other peptide or polypeptide sequence.

In another aspect, the invention provides an isolated and/or exogenous polynucleotide encoding the Fab fragment or derivative thereof of the invention, or a heavy or light chain variable region thereof.

In another aspect, the invention provides a vector comprising the polynucleotide of the invention.

In another aspect, the invention provides a host cell comprising the Fab fragment or derivative thereof of the invention, the polynucleotide of the invention, or the vector of the invention.

In a further aspect, the invention provides a method of screening for a Fab fragment or derivative thereof that binds to a target molecule, the method comprising contacting a Fab fragment or derivative thereof of the invention with the target molecule, and determining whether the Fab fragment or derivative thereof binds to the target molecule. In such methods, it is preferred if a polynucleotide encoding the Fab fragment or derivative thereof is expressed in a host cell or in a cell-free expression system. When expressing the Fab fragment or derivative thereof within a cell, this expression may take place in the cytoplasm and/or periplasm of a host cell, such as a bacterial cell, a yeast cell or a mammalian cell.

In a preferred embodiment, the host cell is a bacterial cell and the method comprises:

a) culturing a bacterial cell comprising a polynucleotide encoding the Fab fragment or derivative thereof of the invention such that the Fab fragment or derivative thereof is produced,

b) permeabilising the bacterial cell, wherein the polynucleotide and the Fab fragment or derivative thereof is retained inside the permeabilised bacterial cell,

c) contacting the permeabilised bacterial cell with the target molecule such that it diffuses into the permeabilised bacterial cell, and

d) determining whether the Fab fragment or derivative thereof of the invention binds to the target molecule.

The screening methods of the invention can be performed using any of the Fab fragments or derivatives thereof described herein. Preferably, the screening methods of the invention comprise screening a library of the invention. Thus, the screening methods may comprise expressing a Fab fragment or derivative thereof or polynucleotide library of the invention and identifying Fab fragments or derivatives thereof within those libraries that bind to a target molecule. Preferably, such screening methods are performed under reducing conditions. For example, such methods can be performed in a host cell. In a preferred embodiment, such methods are performed in the cytoplasm of a host cell. Preferably, the host cell is a bacterial cell, such as a gram negative bacterial cell. In a preferred embodiment, the bacterial cell is an E. coli cell.

In a further aspect, the invention provides a host cell library comprising a plurality of host cells comprising a Fab fragment or derivative thereof of the invention, wherein at least one host cell comprises a Fab fragment or derivative thereof that differs from a Fab fragment or derivative thereof present in another host cell in the library in the sequence of amino acids present in one or more CDRs in the V_{H} and/or V_{L} variable domains. One or more host cells in the host cell library of the invention may comprise one or more polynucleotides encoding the Fab fragment or derivative thereof of the invention. For example, a host cell in the host cell library may contain one polynucleotide encoding the V_{H} and another polynucleotide encoding the V_{L}.

In one embodiment, the Fab fragment or derivative thereof is in the cytoplasm of the host cell.

In one particular embodiment, the host cell is selected from a bacterial cell, yeast or mammalian cell.

In another aspect, the invention provides a composition comprising the Fab fragment or derivative thereof, the polynucleotide and/or the vector of the invention, and a pharmaceutically acceptable carrier.

In another aspect, the invention provides a kit comprising the Fab fragment or derivative thereof of the invention, the polynucleotide of the invention and/or the vector of the invention, and an agent capable of permeabilising a bacterial cell.

In a further aspect, the invention provides the use of the Fab fragment or derivative thereof of the invention in therapeutic or diagnostic applications.

In yet another aspect, the invention provides a non-filamentous phage displaying a Fab fragment or derivative thereof of in the cytoplasm of a cell.

In one embodiment, the non-filamentous phage displays a Fab or derivative thereof of the invention in the cytoplasm of the cell.

In one embodiment, the phage is a lambdoid phage.

In another embodiment, the phage displays the Fab fragment or derivative thereof in the in the cytoplasm of a cell selected from a bacterial, yeast or mammalian cell. Preferably, the bacterial cell is a Gram negative bacterial cell. In one particular embodiment, the bacterial cell is an E. coli cell.

In one embodiment, the antibody heavy chain variable region and/or antibody light chain variable region is fused or linked to a coat protein of the non-filamentous phage.

In another embodiment, the phage is a lysis-defective phage.

In another aspect, the present invention provides a host cell comprising a non-filamentous phage displaying a Fab or derivative thereof. In one embodiment, the host cell comprises a non-filamentous phage displaying a Fab or derivative thereof according to the invention.

In one embodiment, the cell is selected from a bacterial, yeast or mammalian cell. In one particular embodiment, the bacterial cell is an E. coli cell.

In yet another aspect, the present invention provides a method for screening a polynucleotide library for nucleotide sequences encoding a Fab fragment or derivative thereof of the invention that binds a target molecule, the method comprising:

a) transforming a host cell with a polynucleotide encoding the Fab fragment or derivative thereof of the invention,

b) cultivating the transformed host cell under conditions suitable for expression and assembly of a non-filamentous phage particle comprising a coat protein fused or linked to the heavy chain variable region and/or antibody light chain variable region of the Fab fragment or derivative thereof; and

c) determining whether the Fab fragment or derivative thereof of the invention binds to the target molecule.

In embodiment, the host cell is a bacterial cell, yeast or mammalian cell.

In another embodiment, the host cell is permeabilised.

In yet another embodiment, the target molecule diffuses into the host cell.

In one particular embodiment, the host cell is a permeabilised Gram negative bacterial cell.

In aspects of the invention relating to a non-filamentous phage displaying a Fab or derivative thereof of the invention, or which require a non-filamentous phage particle comprising a coat protein fused or linked to the heavy chain variable region and/or antibody light chain variable region of the Fab fragment or derivative thereof of the invention, the phage may be any non-filamentous phage including a non-filamentous filamentous phage selected from phiX174, T1, T2, T3, T4, T5, T6, T7 bacteriophages, lambdoid bacteriophages, N15 phage, Mu phage, P2 phage, phage 186 and the P2 satellite phage, P4.

In one embodiment, the non-filamentous phage is a lambdoid phage selected from lambda phage, P22 phage, HK97, HK022, 933W, 434, 21, 82, and phi80 phage.

In yet another embodiment, the non-filamentous phage is a lysis-defective phage.

In the preceding aspects of the invention, the non-filamentous phage displays a Fab fragment or derivative thereof in the cytoplasm of a cell. In one particular embodiment, the Fab fragments or derivatives thereof can be retained in a soluble fraction of a cell lysate at a level of at least 25%. In another embodiment, the Fab fragments or derivatives thereof can be retained in a soluble fraction of a cell lysate at a level of at least 50%, or at least 75%.

As will be apparent, preferred features and characteristics of one aspect of the invention are applicable to any other aspects of the invention, mutatis mutandis.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting Examples and with reference to the accompanying figures.

### BRIEF DESCRIPTION OF THE ACCOMPANYING DRAWINGS

FIG. 1 shows the typical appearance of a well-expressed, soluble scFv clone (1A, and inset), along with a well-expressed, but insoluble scFv clone (1B, and inset).

FIG. 2 shows a multiple alignment of selected soluble clones that have high similarity, or total identity, to the VL genes IGLV3-1, IGLV3-21 and IGLV6-57.

FIG. 3 demonstrates the behaviour of two clones, one IGLV3-1 and one IGLV3-21, with expression at increasing temperatures.

FIG. 4 demonstrates the solubility of an IGLV3-1 clone when expressed in the E. coli cytosol at 25° C. The scFv::I27::FLAG fusion protein is entirely in the soluble (S) fraction.

FIG. 5 demonstrates the thermostability behaviour of the original clone (#8.93) with replacement of the λJ region for J1 or J2.

FIG. 6A demonstrates the solubility and high expression of 4 independent clones with the IGLV3-1 CDR3 diversified.

FIG. 6B demonstrates a sample of the entire population of clones with the IGHV3-23 CDR3 diversified.

FIG. 7 illustrates exemplary CDRs (in bold and/or underlined) in preferred variable regions described herein.

FIG. 8 illustrates an example of a polynucleotide sequence encoding an IGLV3-1::IGHV3-23 scaffold with variable CDR3 regions, and the corresponding, translated amino acid sequence. CDRs are underlined and in bold type. A peptide linker sequence is italicized.

FIG. 9 shows the SNAP ligand-labeled IGLV3-1::IGHV3-23 scFv library, demonstrating the high frequency of soluble library members.

FIG. 10 shows the isolation of mAG-binding scFvs from a RED screen. Clone 34 was positive for mAG binding. Clone 25 was negative.

FIG. 11 illustrates the soluble nature of the IGLV3-1::IGHV3-23 scFv scaffold with the entirety of the α-mAG scFv isolated from the RED screen cloned as a C-terminal His6 FLAG fusion protein present in the soluble fraction (S), with no protein in the insoluble fraction (P). Detection was using an α-FLAG monoclonal antibody.

FIG. 12 shows the binding of mAG by the α-mAG scFv His6FLAG fusion bound to IMAC Ni-sepharose.

FIG. 13 demonstrates the specificity of the α-mAG scFv interaction for mAG by a ‘pull-down’ of unpurified mAG from E. coli lysate. α-mAG scFv His6 FLAG was bound to IMAC Ni-sepharose resin with the addition of mAG in total E. coli cell lysate (lanes 6 and 7) resulting in the binding of a protein of the expected size of mAG (˜26 kD).

FIG. 14 shows a screen-grab (Top) from the FACS stage of the ‘doped’ mAG library screen using the encapsulated lysis-defective bacteriophage displaying the gpD::α-mAG scFv fusion protein. The mAG-positive cells containing encapsulated phage are in the right gate. The bacteriophage recovered from the FACS screen were induced for bacteriophage replication and gpD::α-mAG expression and labeled with mAG using the RED method (Bottom).

FIG. 15 shows mAG1 labeling of α-mAG1 Fab expressing cells. E. coli cells expressing the α-mAG1 Fab::PG::SNAP::DBP fusion protein were detergent permeabilised as described and co-labeled with the fluorescent mAG1 target (panel 1) and the SNAP surface 549 (panel 2) label. The peripheral cell labeling with both mAG1 and SNAP 549 indicates soluble, functional Fab expression.

FIG. 16 shows eGFP labeling of α-eGFP Fab expressing cells. E. coli cells expressing a α-eGFP Fab::PG::SNAP::DBP fusion protein were detergent permeabilised as described and co-labeled with the fluorescent eGFP target (lower panel 1) and the SNAP surface 549 (lower panel 2) label. The peripheral cell labeling with both mAG1 and SNAP 549 indicates soluble, functional Fab expression. The upper panels demonstrates a camelid α-eGFP::PG::SNAP::DBP control for eGFP binding.

FIG. 17 shows that germline IGLV genes identified as being cytoplasmically soluble when paired with germline IGHV3-23 were similarly soluble when expressed as Fabs in the E. coli cytoplasm. Both light and heavy chains were tagged with the FLAG epitope and detected in the soluble (S) and insoluble (P) cellular fractions by Western blot using anti-FLAG.

FIG. 18 shows the cytoplasmic solubility of an anti-mAG1 Fab fused via the heavy chain C-terminus to the vertebrate calmodulin gene. The fusion protein is detected via a FLAG epitope fused to the extreme C-terminus of the calmodulin gene. The insoluble cellular material was loaded into lane 1 (InS) and the soluble extract loaded into lane 2 (S).

### KEY TO THE SEQUENCE LISTING

SEQ ID NO: 1—polynucleotide sequence encoding IGHV3-23 (NCBI Ref. NT_{—}026437.12).
SEQ ID NO: 2—polynucleotide sequence encoding IGHV3-23, excluding introns.
SEQ ID NO: 3—amino acid sequence of IGHV3-23
SEQ ID NO: 4—polynucleotide sequence encoding IGLV3-1 (NCBI Ref. NT_{—}011520.12).
SEQ ID NO: 5—polynucleotide sequence encoding IGLV3-1, excluding introns.
SEQ ID NO: 6—amino acid sequence of IGLV3-1
SEQ ID NO: 7—polynucleotide sequence encoding IGLV3-21 (NCBI Ref. NT_{—}011520.12)
SEQ ID NO: 8—polynucleotide sequence encoding IGLV3-21, excluding introns.
SEQ ID NO: 9—amino acid sequence of IGLV3-21
SEQ ID NO: 10—polynucleotide sequence encoding IGLV6-57 (NCBI Reference: NW_{—}001838745.1)
SEQ ID NO: 11—polynucleotide sequence encoding IGLV6-57, excluding introns.
SEQ ID NO: 12—amino acid sequence of IGLV6-57
SEQ ID NO: 13—polynucleotide sequence encoding IGLV1-51 (NCBI Reference Sequence: NT_{—}011520.12)
SEQ ID NO: 14—polynucleotide sequence encoding IGLV1-51, excluding introns.
SEQ ID NO: 15—amino acid sequence of IGLV1-51
SEQ ID NO: 16—polynucleotide sequence encoding IGLV1-40 (NCBI Reference Sequence: NT_{—}011520.12)
SEQ ID NO: 17—polynucleotide sequence encoding IGLV1-40, excluding introns.
SEQ ID NO: 18—amino acid sequence of IGLV1-40
SEQ ID NO: 19—polynucleotide sequence encoding IGLV1-44 (NCBI Reference Sequence: NT_{—}011520.12)
SEQ ID NO: 20—polynucleotide sequence encoding IGLV1-44, excluding introns.
SEQ ID NO: 21—amino acid sequence of IGLV1-44
SEQ ID NO: 22—polynucleotide sequence encoding IGLV1-47 (NCBI Reference Sequence: NT_{—}011520.12)
SEQ ID NO: 23—polynucleotide sequence encoding IGLV1-47, excluding introns.
SEQ ID NO: 24—amino acid sequence of IGLV1-47
SEQ ID NO: 25—polynucleotide sequence encoding IGLV3-19 (NCBI Reference Sequence: NT_{—}011520.12)
SEQ ID NO: 26—polynucleotide sequence encoding IGLV3-19, excluding introns.
SEQ ID NO: 27—amino acid sequence of IGLV3-19
SEQ ID NO: 28—CDR variant sequence
SEQ ID NO: 29—Alternative CDR variant sequence
SEQ ID NO: 30—Lamda J region J1
SEQ ID NO: 31—Lamda J region J2
SEQ ID NO: 32—Lamda J region J3
SEQ ID NO: 33—Lamda J region J4
SEQ ID NO: 34—Lamda J region J5
SEQ ID NO: 35—Lamda J region J6
SEQ ID NO: 36—Lamda J region J7
SEQ ID NO: 37—Hybrid J region sequence
SEQ ID NO: 38—PCR primer
SEQ ID NO: 39—Translated sequence
SEQ ID NO: 40—PCR primer
SEQ ID NO: 41—Translated sequence
SEQ ID NO: 42—Polynucleotide sequence encoding an IGLV3-1::IGHV3-23 scaffold with variable CDR3 regions
SEQ ID NO: 43—Amino acid sequence encoded by the polynucleotide of SEQ ID NO: 42
SEQ ID NO: 44—Framework sequence of IGLV3-1 and the J region of IGHV3-23
SEQ ID NO: 45—Intervening sequence
SEQ ID NO: 46—CDR3 loop L1
SEQ ID NO: 47—CDR3 loop H1
SEQ ID NO: 48—CDR3 loop L2
SEQ ID NO: 49—CDR3 loop H2
SEQ ID NO: 50—CDR3 loop L3
SEQ ID NO: 51—CDR3 loop H3
SEQ ID NO: 52—CDR3 loop L4
SEQ ID NO: 53—CDR3 loop H4
SEQ ID NO: 54—CDR3 loop L5
SEQ ID NO: 55—CDR3 loop H5
SEQ ID NO: 56—CDR3 loop L6
SEQ ID NO: 57—CDR3 loop H6
SEQ ID NO: 58—CDR3 loop L8
SEQ ID NO: 59—CDR3 loop H8
SEQ ID NO: 60—CDR3 loop L9
SEQ ID NO: 61—CDR3 loop H9
SEQ ID NO: 62—CDR3 loop L10
SEQ ID NO: 63—CDR3 loop H10
SEQ ID NO: 64—Anti-mAG-BioHis6 scFv sequence
SEQ ID NO: 65—Wildtype human IGLV 3-1
SEQ ID NO: 66—Soluble clone 8.93
SEQ ID NO: 67—Soluble clone 8.184
SEQ ID NO: 68—Soluble clone 8.174
SEQ ID NO: 69—Soluble human IGLV 3-21 clone 8.186
SEQ ID NO: 70—Soluble human IGLV 3-21 clone 8.39
SEQ ID NO: 71—Wildtype human IGLV 3-21
SEQ ID NO: 72—Soluble human IGLV 3-21 clone 9.19
SEQ ID NO: 73—Wildtype human IGLV 6-57
SEQ ID NO: 74—Soluble clone 16.26
SEQ ID NO: 75—Soluble clone 16.1
SEQ ID NO: 76—Soluble clone 16.121
SEQ ID NO: 77—IGLC2 amino acid sequence
SEQ ID NO: 78—IGHG3 amino acid sequence
SEQ ID NO: 79—IGLV3-1::IGLC2, IGHV3-23::CH1 IGHG3 polynucleotide sequence
SEQ ID NO: 80—IGLV3-1::IGLC2, IGHV3-23::CH1 IGHG3 amino acid sequence
SEQ ID NO: 81—IGLV3-1::IGLC2, IGHV3-23::CH1 IGHG3 amino acid sequence
SEQ ID NO: 82—α-mAG1 V_{H}::CH1 IGHG3 amino acid sequence
SEQ ID NO: 83—α-mAG1 Fab polynucleotide sequence
SEQ ID NO: 84—α-mAG1 Fab::PG::SNAP::DBP fusion amino acid sequence
SEQ ID NO: 85—FLAG epitope
SEQ ID NO:86—V_{L} CDR3+J region sequence
SEQ ID NO:87—IGLV6-57/IGHV3-23::CaM Fab sequence
SEQ ID NO:88—Fab heavy chain::calmodulin fusion
SEQ ID NO:89—gpD::CBD fusion

### DETAILED DESCRIPTION

### General Techniques and Definitions

Unless specifically defined otherwise, all technical and scientific terms used herein shall be taken to have the same meaning as commonly understood by one of ordinary skill in the art (e.g., in protein chemistry, biochemistry, cell culture, molecular genetics, microbiolgy, and immunology).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, 3^{rd} edn, Cold Spring Harbour Laboratory Press (2001), R. Scopes, Protein Purification—Principals and Practice, 3^{rd} edn, Springer (1994), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The terms “polypeptide”, “protein” and “peptide” are generally used interchangeably herein. As used herein, the term “exogenous polypeptide” refers to a polypeptide encoded by an exogenous polynucleotide. The term “exogenous polynucleotide” as used herein refers to a polynucleotide which is foreign to the cell into which it has been introduced, or that the sequence is homologous to a sequence in the cell into which it is introduced but in a position within the host cell nucleic acid in which the polynucleotide is not normally found.

The skilled artisan will be aware that an antibody is generally considered to be a protein that comprises a variable region made up of a plurality of polypeptide chains, e.g., a light chain variable region (V_{L}) and a heavy chain variable region (V_{H}). An antibody may also comprise constant domains, which can be arranged into a constant region or constant fragment or fragment crystallisable (Fc). Antibodies can bind specifically to one or a few closely related antigens. Full-length antibodies generally comprise two heavy chains (˜50-70 kD) covalently linked and two light chains (˜23 kD each). A light chain generally comprises a variable region and a constant domain and in mammals is either a κ light chain or a λ light chain. A heavy chain generally comprises a variable region and one or two constant domain(s) linked by a hinge region to additional constant domain(s). Heavy chains of mammals are of one of the following types α, δ, ε, γ, or μ. Each light chain is also covalently linked to one of the heavy chains. For example, the two heavy chains and the heavy and light chains may be held together by inter-chain disulfide bonds and/or by non-covalent interactions. The number of inter-chain disulfide bonds (if present) can vary among different types of antibodies. Each chain has an N-terminal variable region (V_{H} or V_{L} wherein each are 110 amino acids in length) and one or more constant domains at the C-terminus. The constant domain of the light chain (C_{L} which is ˜110 amino acids in length) is often aligned with and disulfide bonded to the first constant domain of the heavy chain (C_{H} which is ˜330-440 amino acids in length). The light chain variable region is often aligned with the variable region of the heavy chain. The antibody heavy chain can comprise 2 or more additional C_{H} domains (such as, C_{H}2, C_{H}3 and the like) and can comprise a hinge region that can be identified between the C_{H}1 and Cm constant domains. Antibodies can be of any type (e.g., IgG, IgE, IgM, IgD, IgA, and IgY), class (e.g., IgG₁, IgG₂, IgG₃, IgG₄, IgA₁ and IgA₂) or subclass. Preferably, the antibody is a murine (mouse or rat) antibody or a primate (preferably human) antibody.

A “Fab fragment” consists of a monovalent antigen-binding fragment of an antibody, and can be produced, for example, by digestion of a whole antibody with the enzyme papain, to yield a fragment consisting of an intact light chain and a portion of a heavy chain or can be produced using recombinant means. A “Fab′ fragment” of an antibody can be obtained, for example, by treating a whole immunoglobulin with pepsin, followed by reduction, to yield a molecule consisting of an intact light chain and a portion of a heavy chain. Two Fab′ fragments are obtained per antibody treated in this manner. A Fab′ fragment can also be produced by recombinant means. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fab₂” fragment is a recombinant fragment comprising two Fab fragments linked using, for example a leucine zipper or a C_{H}3 domain.

As used herein, the term “Fab fragment or derivative thereof” includes reference to Fab, F(ab′)2, Fab′ and other Fab-like molecules, as well as to bi-specific and multispecific molecules. For example the Fab fragment or derivative thereof may be a fusion protein that is fused to a second polypeptide or linker domain. The second polypeptide to which the Fab fragment or derivative thereof is fused may be, for example, an antigen binding polypeptide or an enzyme. In one embodiment, the Fab fragment or derivative thereof comprises one or more fusion moieties which bind to one or more additional target antigens.

As used herein, the term “variable region” refers to the portions of the light and heavy chains of an antibody as defined herein that includes amino acid sequences of CDRs; i.e., CDR1, CDR2, and CDR3, and framework regions (FRs). V_{H} refers to the variable region of the heavy chain. V_{L} refers to the variable region of the light chain.

As used herein, the term “scaffold region” refers to all the variable region residues other than the CDR residues.

As used herein, the term “framework region” (FR) will be understood to mean a contiguous sequence of variable region residues other than the CDR residues. Thus, all of the FRs together make up the “scaffold region”. Each variable region of a naturally-occurring antibody typically has four FRs, identified as FR1, FR2, FR3 and FR4. If the CDRs are defined according to Kabat, exemplary light chain FR (LCFR) residues are positioned at about residues 1-23 (LCFR1), 35-49 (LCFR2), 57-88 (LCFR3), and 98-107 (LCFR4). Note that κLCFR1 does not comprise residue 10, which is included in κLCFR1. Exemplary heavy chain FR (HCFR) residues are positioned at about residues 1-30 (HCFR1), 36-49 (HCFR2), 66-94 (HCFR3), and 103-113 (HCFR4).

As used herein, the term “complementarity determining regions” (CDRs; i.e., CDR1, CDR2, and CDR3 or hypervariable region) refers to the amino acid residues of an immunoglobulin variable region the presence of which are necessary for antigen binding. Each variable region typically has three CDR regions identified as CDR1, CDR2 and CDR3. Each CDR may comprise amino acid residues from a “complementarity determining region” as defined by Kabat (1987 and/or 1991). For example, in a heavy chain variable region CDRH1 is between residues 31-35, CDRH2 is between residues 50-65 and CDRH3 is between residues 95-102. In a light chain CDRL1 is between residues 24-34, CDRL2 is between residues 50-56 and CDRL3 is between residues 89-97. These CDRs can also comprise numerous insertions, e.g., as described in Kabat (1987 and/or 1991).

The term “constant region” (CR or fragment crystalizable or Fc) as used herein, refers to a portion of an antibody comprising at least one constant domain and which is generally (though not necessarily) glycosylated and which binds to one or more receptors and/or components of the complement cascade (e.g., confers effector functions). The heavy chain constant region can be selected from any of the five isotypes: α, δ, ε, γ, or μ. Furthermore, heavy chains of various subclasses (such as the IgG subclasses of heavy chains) are responsible for different effector functions and thus, by choosing the desired heavy chain constant region, proteins with desired effector function can be produced. Preferred heavy chain constant regions are gamma 1 (IgG1), gamma 2 (IgG2) and gamma 3 (IgG3).

A “constant domain” is a domain in an antibody the sequence of which is highly similar in antibodies of the same type, e.g., IgG or IgM or IgE. A constant region of an antibody generally comprises a plurality of constant domains, e.g., the constant region of γ, α and δ heavy chains comprise three constant domains and the Fc of γ, α and δ heavy chains comprise two constant domains. A constant region of μ and ε heavy chains comprises four constant domains and the Fc region comprises two constant domains.

A “single chain Fv” or “scFv” is a recombinant molecule containing the variable region fragment (Fv) of an immunoglobulin in which the variable region of the light chain and the variable region of the heavy chain are covalently linked by a suitable, flexible polypeptide linker. A detailed discussion of exemplary Fv containing polypeptides falling within the scope of this term is provided herein below.

As used herein, the term “antigen binding site” shall be taken to mean a structure formed by a polypeptide that is capable of specifically binding to an antigen. The antigen binding site need not be a series of contiguous amino acids, or even amino acids in a single polypeptide chain. For example, in a Fv produced from two different polypeptide chains the antigen binding site is made up of a series of regions of a V_{L} and a V_{H} that interact with the antigen and that are generally, however not always in the one or more of the CDRs in each variable region.

Any amino acid positions assigned to CDRs and FRs herein are defined according to Kabat (1987 and 1991). The skilled artisan will be readily able to use other numbering systems in the performance of this invention, e.g., the hypervariable loop numbering system of Chothia and Lesk (1987 and/or 1989) and/or Al-Lazikani et al. (1997).

The skilled artisan will be aware that a “disulphide bond” is a covalent bond formed by coupling of thiol groups. The bond is also called an SS-bond or disulfide bridge. In polypeptides, a disulphide bond generally occurs between the thiol groups of two cysteine residues.

The skilled artisan will also be aware that the term “non-reducing conditions” includes conditions sufficient for oxidation of sulfhydryl (—SH) groups in a protein, e.g., permissive for disulphide bond formation. Accordingly, the term “reducing conditions” includes conditions which are not sufficient for oxidation of sulfhydryl (—SH) groups in a protein, e.g., not permissive for disulphide bond formation.

As used herein, the term “antigen” shall be understood to mean any composition of matter against which an antibody response can be raised. Exemplary antigens include proteins, peptides, polypeptides, carbohydrates, phosphate groups, phosphor-peptides or polypeptides, glyscosylated peptides or peptides, etc.

The description and definitions of variable regions and parts thereof, immunoglobulins, antibodies and fragments thereof herein may be further clarified by the discussion in Kabat (1987 and/or 1991), Bork et al., (1994) and/or Chothia and Lesk (1987 and 1989) or Al-Lazikani et al., (1997).

As used herein, the terms “conjugate”, “conjugated” or variations thereof are used broadly to refer to any form to covalent or non-covalent association between a compound useful in the methods disclosed herein and another agent.

The term “lysis-defective phage” as used herein does not include reference to a phage that does not normally have a lytic stage in its lifecycle, hence the skilled person will understand that it does not include reference to phage that are released from a bacterial cell by extrusion, for example filamentous phage such as M13, f1 or f2, or that are released from a bacterial cell by budding.

Examples of lytic phages that may be modified to remove the lytic stage from their life-cycle so as to produce a lysis-defective phage include phiX174, T1, T2, T3, T4, T5, T6 and T7 bacteriophages. Examples of lysogenic phage which may be modified so as to remove the lytic stage of their life-cycle include lambda phage, N15 phage, P22 phage, Mu phage, P2 phage, phage 186 and the P2 satellite phage, P4 (Lindqvist et al., 1993; Ziermann et al., 1994; Liu et al., 1997; and Briani et al., 2001).

The skilled person will understand that some temperate phages that are capable of packaging a polynucleotide in a bacterial cell require the presence of another phage, for example, a helper phage, in order to undergo polynucleotide packaging and/or for bacterial cell lysis to occur. An example of this relationship is the P2 phage and its satellite phage, P4. The requirement for the presence of a helper phage for polynucleotide packaging and/or for bacterial cell lysis is known as a helper-phage system. In a helper-phage system, the activity of a helper-phage, or of phage polypeptides (i.e. “activator proteins”), induces another phage to undergo polynucleotide packaging and/or cause bacterial cell lysis. Thus, the skilled person will understand that while a polynucleotide may be packaged into one phage (i.e. one phage in a helper-phage system), the activity of another phage (i.e. a helper-phage) may be required to lyse the bacterial cell in which both the phages are present. For use in some embodiments of the method of the present invention, the phage which would normally provide the lytic activity is modified so that it is no longer capable of lysing a bacterial cell. Accordingly, the term “lysis-defective phage” as used herein also refers to a phage into which a polynucleotide is packaged, wherein the phage would normally rely on a second phage to provide lytic activity, but in which the second phage has been modified so that it is no longer capable of lysing a Gram-negative bacterial cell.

The term “and/or”, e.g., “X and/or Y” shall be understood to mean either “X and Y” or “X or Y” and shall be taken to provide explicit support for both meanings or for either meaning.

The term “about” as used herein refers to a range of +/−5% of the specified value.

As will be understood from the following description, the present inventors have applied protein display methods to identify Fab fragments or derivatives thereof that can be expressed in soluble form in the cellular cytoplasm and that demonstrate particular effective levels of solubility, thermostability, and tolerance to CDR diversification.

### Retained Encapsulated Display (RED):

The present inventors have identified polypeptides that can be expressed in soluble form in the cellular cytoplasm and that demonstrate surprising levels of solubility, thermostability, and tolerance to CDR diversification using the method of Retained Encapsulated Display (RED). RED is a protein display platform for gram-negative bacteria that is described in WO 2011/075761 (the content of which is incorporated by reference in its entirety). In RED the protein to be displayed is expressed in either the periplasm or cytoplasm of the cell. The cellular membranes are then permeabilised with detergent or organic solvents while the cell wall is left intact. The display protein is retained by the cell wall, either through fusion to proteins that increase its molecular size to above the porosity limit for the cell wall (e.g. fusion to tetramer monomers), or through fusion to protein domains that bind either DNA, the cell wall itself, or both. The phenotype-genotype linkage required for a display system is provided through the co-retention of the plasmid and genomic DNA within the cell wall of the permeabilised cell.

### Polypeptides:

The human antibody repertoire contains both functional and pseudogene variable regions (summarized by Lefranc, 2000). These may be cloned as exons from either genomic DNA in non-immune lineages, or from mRNA sourced from immune cells that have undergone V(D)J recombination, in order to prepare a genetic construct which can be used to express the antibody. During such a process, the variable domains of the light and heavy chains may be cloned as either a monomeric scFv, or in arrangements that form bivalent or higher-order valencies. The constant regions may also be cloned downstream of the variable domains to create Fab or full-length antibodies.

In all forms, to attain the correct fold and maintain stability and solubility during the production of an antibody, the genetic constructs encoding the antibody must almost always be expressed under conditions such that intra-domain disulphide bonds may form between the β sheets (i.e., under non-reducing conditions). Thus, in mammalian cells, antibodies are inserted into the endoplasmic reticulum (ER) and Golgi for secretion or membrane insertion. If expressed in a bacterial host such as E. coli they must be directed to the periplasmic space where the disulphide bond chaperones DsbA, B and C reside. If an antibody is expressed in a non-oxidising environment, (such as in the cellular cytoplasm) the lack of stabilizing disulphide bonds results in misfolding and degradation or, if expressed at a high level in the E. coli cytoplasm, aggregation as a subcellular inclusion body.

The present inventors have determined that Fab fragments or derivatives thereof comprising certain antibody variable region scaffolds are capable of forming an antigen binding site even when the Fab fragments or derivatives thereof are expressed in a non-oxidising (reducing) environment.

Accordingly, the invention provides an isolated and/or recombinant Fab fragment or derivative thereof comprising an antibody heavy chain variable region (V_{H}) of the V_{H}3 family of immunoglobulin variable domains and an antibody light chain variable region (V_{L}) of the V_{L}λ1, 3 or 6 families of immunoglobulin variable domains, wherein the V_{H} and the V_{L} are capable of forming an antigen-binding site.

The Fab fragments or derivatives thereof of the invention preferably comprise a scaffold region of a V_{H} of the V_{H}3 family of immunoglobulin variable and/or a scaffold region of a V_{L} of the V_{L}λ1, 3 or 6 families of immunoglobulin variable domains. Thus, the Fab fragments or derivatives thereof of the invention preferably comprise all of the amino acid residues of any of the variable regions disclosed herein, excluding CDR residues. The CDR residues can readily be identified by the person skilled in the art, with reference to the discussion in Kabat (1987 and/or 1991), Bork et al., (1994) and/or Chothia and Lesk (1987 and 1989) or Al-Lazikani et al., (1997). Thus, the Fab fragments or derivatives thereof of the invention can comprise all of the FRs of any variable region disclosed herein. The Fab fragments or derivatives thereof may further comprise one or more of the CDRs of the variable regions disclosed herein. The Fab fragments or derivatives thereof may also comprise one or more CDRs which are not present in the variable regions disclosed herein. Thus, one or more CDRs from a different source can be inserted into the scaffold region of the variable regions disclosed herein. A further discussion of such possibilities is included herein, below.

In a preferred embodiment, Fab fragments or derivatives thereof of the invention comprise a scaffold region of a V_{H} of the V_{H}3 family of immunoglobulin variable and a V_{L} of the V_{L}λ1, 3 or 6 families of immunoglobulin variable domains. In further preferred embodiments, the Fab fragment or derivative thereof of the invention comprise a scaffold region of IGHV3-23 and a scaffold region of any one of IGLV1-40, IGLV1-44, IGLV1-47, IGLV1-51, IGLV3-1, IGLV3-19, IGLV3-21, and IGLV6-57. Most preferably, the Fab fragments or derivatives thereof of the invention comprise a scaffold region of IGHV3-23 and a scaffold region of IGLV3-1.

The Fab fragments or derivatives thereof of the invention may be defined in terms of their percentage identity to a reference sequence. This percentage identity may be calculated by any suitable method known in the art. Several algorithms for comparing aligned sequences are known, and can be used to determine the percentage identity of a polypeptide of the invention to a reference sequence. For example, amino acid and polynucleotide sequences can be compared manually or by using computer-based sequence comparison and identification tools that employ algorithms such as BLAST (Basic Local Alignment Search Tool; Altschul et al., 1993); see also www.ncbi.nlm.nih.gov/BLAST/), the Clustal method of alignment (Higgins and Sharp, 1989) and others, wherein appropriate parameters for each specific sequence comparison can be selected as would be understood by a person skilled in the art.

Preferably, the Fab fragment or derivative thereof of the invention is an isolated and/or recombinant polypeptide. The term “isolated” or “purified” as used herein is intended to mean a polypeptide that has generally been separated from the lipids, nucleic acids, other polypeptides and peptides, and other contaminating molecules with which it is associated in its native state. Preferably, the isolated polypeptide is at least 60% free, more preferably at least 75% free, and more preferably at least 90% free from other components with which it is naturally associated.

The term “recombinant” in the context of a polypeptide refers to the polypeptide when produced by a cell, or in a cell-free expression system, in an altered amount or at an altered rate compared to its native state. In one embodiment the cell is a cell that does not naturally produce the polypeptide. However, the cell may be a cell which comprises a non-endogenous gene that causes an altered, preferably increased, amount of the peptide to be produced. A recombinant polypeptide (i.e., a Fab fragment or derivative thereof) as described herein includes polypeptides which have not been separated from other components of the transgenic (recombinant) cell or cell-free expression system in which it is produced, and polypeptides produced in such cells or cell-free systems which are subsequently purified away from at least some other components.

The Fab fragment or derivative of the invention preferably comprises amino acid sequences which are derived from a murine (mouse or rat) antibody or a primate (preferably human) antibody. Thus, the variable regions and/or scaffold regions included in the polypeptides of the invention may be murine (mouse or rat) or primate (preferably, human) variable regions and/or scaffold regions.

Preferably, the Fab fragments or derivatives thereof of the invention are soluble. Methods for determining the solubility of a polypeptide are well known in the art, e.g., as described by J. Sambrook et al., Molecular Cloning: A Laboratory Manual, 3^{rd} edn, Cold Spring Harbour Laboratory Press (2001). The polypeptides may be determined to be soluble if, for example, they cannot be separated from a lysed and/or permeabilised cell fraction by physical separation (e.g. by centrifugation). In addition, the Fab fragments or derivatives thereof of the invention may be determined to be soluble if they do not form inclusion bodies in cellular cytoplasm. Thus, the Fab fragments or derivatives thereof may be considered to be soluble if, when they are expressed in a host cell, they are retained in a soluble fraction produced after lysis of the host cell by any suitable mechanical, detergent and/or enzymatic methods. Suitable mechanical methods include, for example, the use of sonication. Suitable detergent methods include, for example, the use of n-Octyl-β-D-Thioglucoside (8TGP). Suitable enzymatic methods include, for example, the use of lysozyme. Preferably, the Fab fragments or derivatives thereof of the invention can be retained in a soluble fraction of a cell lysate at a level of at least 25%, such as at least 50%, at least 75%, at least 90%, at least 95%, or at least 95%.

The Fab fragments or derivatives thereof of the invention are preferably capable of stably forming an antigen binding site. Thus, the Fab fragments or derivatives thereof are preferably capable of binding to a target antigen at a level which is sufficient to allow detection of the Fab-antigen complex. Such detection may take place under any suitable experimental conditions, such as at a temperature of at least 5° C., at least 10° C., at least 15° C., at least 20° C., at least 25° C., at least 30° C., at least 35° C., at least 40° C., at least 45° C. or at least 50° C.

### Conjugates

The Fab fragment or derivative thereof of the invention may be conjugated to one or more compounds using any suitable method known in the art. Examples of compounds to which a polypeptide (i.e. Fab fragment or derivative thereof) can be conjugated are selected from the group consisting of a radioisotope, a detectable label, a therapeutic compound, a colloid, a toxin, a nucleic acid, a peptide, a protein, a compound that increases the half life of the protein in a subject and mixtures thereof. Exemplary therapeutic agents include, but are not limited to an anti-angiogenic agent, an anti-neovascularization and/or other vascularization agent, an anti-proliferative agent, a pro-apoptotic agent, a chemotherapeutic agent or a therapeutic nucleic acid.

A toxin includes any agent that is detrimental to (e.g. kills) cells. For a description of these classes of drugs which are known in the art, and their mechanisms of action, see Goodman et al., Goodman and Gilman's The Pharmacological Basis of Therapeutics, 8th Ed., Macmillan Publishing Co., 1990. Additional techniques relevant to the preparation of immunoglobulin-immunotoxin conjugates are provided in for instance Vitetta (1993) and U.S. Pat. No. 5,194,594. Exemplary toxins include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232.

Suitable chemotherapeutic agents for forming immunoconjugates comprising polypeptides of the present invention include auristatins and maytansines, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-de-hydrotestosterone, glucocorticoids, procaine, tetracaine, lidocaine, propranolol, and puromycin, antimetabolites (such as methotrexate, 6-mercaptopurine, 6-thioguanine, cytarabine, fludarabin, 5-fluorouracil, decarbazine, hydroxyurea, asparaginase, gemcitabine, cladribine), alkylating agents (such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin), antibiotics (such as dactinomycin (formerly actinomycin), bleomycin, daunorubicin (formerly daunomycin), doxorubicin, idarubicin, mithramycin, mitomycin, mitoxantrone, plicamycin, anthramycin (AMC)).

Examples of suitable angiogenesis inhibitors (anti-angiogenic agents) include, but are not limited to, urokinase inhibitors, matrix metalloprotease inhibitors (such as marimastat, neovastat, BAY 12-9566, AG 3340, BMS-275291 and similar agents), inhibitors of endothelial cell migration and proliferation (such as TNP-470, squalamine, 2-methoxyestradiol, combretastatins, endostatin, angiostatin, penicillamine, SCH66336 (Schering-Plough Corp, Madison, N.J.), R115777 (Janssen Pharmaceutica, Inc, Titusville, N.J.) and similar agents), antagonists of angiogenic growth factors (such as such as ZD6474, SU6668, antibodies against angiogenic agents and/or their receptors (such as VEGF, bFGF, and angiopoietin-1), thalidomide, thalidomide analogs (such as CC-5013), Sugen 5416, SU5402, antiangiogenic ribozyme (such as angiozyme), interferon α (such as interferon α2a), suramin and similar agents), VEGF-R kinase inhibitors and other anti-angiogenic tyrosine kinase inhibitors (such as SU011248), inhibitors of endothelial-specific integrin/survival signaling (such as vitaxin and similar agents), copper antagonists/chelators (such as tetrathiomolybdate, captopril and similar agents), carboxyamido-triazole (CAI), ABT-627, CM101, interleukin-12 (IL-12), IM862, PNU145156E as well as nucleotide molecules inhibiting angiogenesis (such as antisense-VEGF-cDNA, cDNA coding for angiostatin, cDNA coding for p53 and cDNA coding for deficient VEGF receptor-2) and similar agents. Other examples of inhibitors of angiogenesis, neovascularization, and/or other vascularization are anti-angiogenic heparin derivatives and related molecules (e.g., heperinase III), temozolomide, NK4, macrophage migration inhibitory factor (MIF), cyclooxygenase-2 inhibitors, inhibitors of hypoxia-inducible factor 1, anti-angiogenic soy isoflavones, oltipraz, fumagillin and analogs thereof, somatostatin analogues, pentosan polysulfate, tecogalan sodium, dalteparin, tumstatin, thrombospondin, NM-3, combrestatin, canstatin, avastatin, antibodies against other relevant targets (such as anti-alpha-v/beta-3 integrin and anti-kininostatin mAbs) and similar agents.

In one example, a Fab fragment or derivative thereof as described herein according to any embodiment is conjugated or linked to another polypeptide, including another Fab fragment or derivative thereof of the invention or a polypeptide comprising an immunoglobulin variable region, such as an immunoglobulin or a polypeptide derived therefrom. Other proteins are not excluded. Additional proteins will be apparent to the skilled artisan and include, for example, an immunomodulator or a half-life extending protein or a peptide or other protein that binds to serum albumin, amongst others.

Exemplary immunomodulators include cytokines and chemokines. The term “cytokine” is a generic term for proteins or peptides released by one cell population which act on another cell as intercellular mediators. Examples of cytokines include lymphokines, monokines, growth factors and traditional polypeptide hormones.

Included among the cytokines are growth hormones such as human growth hormone, N-methionyl human growth hormone, and bovine growth hormone; parathyroid hormone, thyroxine, insulin, proinsulin, relaxin, prorelaxin, glycoprotein hormones such as follicle stimulating hormone (FSH), thyroid stimulating hormone (TSH) and luteinizing hormone (LH), hepatic growth factor; prostaglandin, fibroblast growth factor, prolactin, placental lactogen, OB protein, tumor necrosis factor-α and -β; mullerian-inhibiting substance, gonadotropin-associated peptide, inhibin, activin, vascular endothelial growth factor, integrin, thrombopoietin (TPO), nerve growth factors such as NGF-B, platelet-growth factor, transforming growth factors (TGFs) such as TGF-α and TGF-β, insulin-like growth factor-I or -II, erythropoietin (EPO), osteoinductive factors, interferons such as interferon-α, -β, or -γ; colony stimulating factors (CSFs) such as macrophage-CSF (M-CSF), granulocyte-macrophage-CSF (GM-CSF); and granulocyte-CSF (G-CSF), interleukins (ILs) such as IL-1, IL-1α, IL-2, IL-3, IL-4, IL-5, IL-6, IL-7, IL-8, IL-9, IL-10, IL-11, IL-12; IL-13, IL-14, IL-15, IL-16, IL-17, IL-18, IL-21 and LIF.

Chemokines generally act as chemoattractants to recruit immune effector cells to the site of chemokine expression. Chemokines include, but are not limited to, RANTES, MCAF, MIP1-alpha or MIP1-Beta. The skilled artisan will recognize that certain cytokines are also known to have chemoattractant effects and could also be classified under the term chemokines.

Exemplary serum albumin binding peptides or protein are described in US20060228364 or US20080260757.

A variety of radionuclides are available for the production of radioconjugated proteins. Examples include, but are not limited to, low energy radioactive nuclei (e.g., suitable for diagnostic purposes), such as ¹³C, ¹⁵N, ²H, ¹²⁵l, ¹²³l, ⁹⁹Tc, ⁴³K, ⁵²Fe, ⁶⁷Ga, ⁶⁸Ga, ¹¹¹In and the like. Preferably, the radionuclide is a gamma, photon, or positron-emitting radionuclide with a half-life suitable to permit activity or detection after the elapsed time between administration and localization to the imaging site. The present invention also encompasses high energy radioactive nuclei (e.g., for therapeutic purposes), such as ¹²⁵l, ¹³¹l, ¹²³, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re. These isotopes typically produce high energy α- or β-particles which have a short path length. Such radionuclides kill cells to which they are in close proximity, for example neoplastic cells to which the conjugate has attached or has entered. They have little or no effect on non-localized cells and are essentially non-immunogenic. Alternatively, high-energy isotopes may be generated by thermal irradiation of an otherwise stable isotope, for example as in boron neutron-capture therapy (Guan et al., 1998).

In another embodiment, the Fab fragment or derivative thereof is conjugated to a “receptor” (such as streptavidin) for utilization in cell pretargeting wherein the conjugate is administered to the patient, followed by removal of unbound conjugate from the circulation using a clearing agent and then administration of a “ligand” (e.g., biotin) that is conjugated to a therapeutic agent (e.g., a radionucleotide).

The Fab fragment or derivative thereof of the present invention can be modified to contain additional nonproteinaceous moieties that are known in the art and readily available. Preferably, the moieties suitable for derivatization of the Fab fragment or derivative thereof are water soluble polymers. Non-limiting examples of water soluble polymers include, but are not limited to, polyethylene glycol (PEG), polyvinyl alcohol (PVA), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol (PPG) homopolymers, prolypropylene oxide/ethylene oxide copolymers, polyoxyethylated polyols (e.g., glycerol; POG), polyvinyl alcohol, and mixtures thereof. Polyethylene glycol propionaldehyde may have advantages in manufacturing due to its stability in water.

The polymer molecules are typically characterized as having for example from about 2 to about 1000, or from about 2 to about 300 repeating units.

For example water-soluble polymers, including but not limited to PEG, poly(ethylene oxide) (PEO), polyoxyethylene (POE), polyvinyl alcohols, hydroxyethyl celluloses, or dextrans, are commonly conjugated to proteins to increase stability or size, etc., of the protein.

PEG, PEO or POE refers to an oligomer or polymer of ethylene oxide. In the case of PEG, these oligomers or polymers are produced by, e.g., anionic ring opening polymerization of ethylene oxide initiated by nucleophilic attack of a hydroxide ion on the epoxide ring. One of the more useful forms of PEG for protein modification is monomethoxy PEG (mPEG).

Particularly preferred compounds for conjugation to the polypeptide of the present invention are set out in Table 1.

TABLE 1
Preferred compounds for conjugation

| Group | Detail |
|---|---|
| Radioisotopes | ¹²³I, ¹²⁵I, ¹³⁰I, ¹³³I, ¹³⁵I, ⁴⁷Sc, ⁷²As, ⁷²Sc, ⁹⁰Y, ⁸⁸Y, |
| (either directly | ⁹⁷Ru, ¹⁰⁰Pd, ^{101m}Rh, ^{101m}Rh, ¹¹⁹Sb, ¹²⁸Ba, ¹⁹⁷Hg, |
| or indirectly) | ²¹¹At, ²¹²Bi, ¹⁵³Sm, ¹⁶⁹Eu, ²¹²Pb, ¹⁰⁹Pd, ¹¹¹In, ⁶⁷Gu, |
| | ⁶⁸Gu, ⁶⁷Cu, ⁷⁵Br, ⁷⁶Br, ⁷⁷Br, ^{99m}Tc, ¹¹C, ¹³N, ¹⁵O, |
| | ¹⁸I, ¹⁸⁸Rc, ²⁰³Pb, ⁶⁴Cu, ¹⁰⁵Rh, ¹⁹⁸Au, ¹⁹⁹Ag or ¹⁷⁷Lu |
| Half life | Polyethylene glycol |
| extenders | Glycerol |
| | Glucose |
| Fluorescent | Phycoerythrin (PE) |
| probes | Allophycocyanin (APC) |
| | Alexa Fluor 488 |
| | Cy5.5 |
| Biologics | Fluorescent proteins such as Renilla luciferase, GFP |
| | Immune modulators |
| | Toxins |
| | An Immunoglobulin |
| | Half life extenders such as albumin |
| Chemo- | Taxol |
| therapeutics | 5-Fluorouracil |
| | Doxorubicin |
| | Idarubicin |

In one example of the invention, a spacer moiety is included between the compound and the polypeptide to which it is conjugated. The spacer moieties of the invention may be cleavable or non-cleavable. For example, the cleavable spacer moiety may be a redox-cleavable spacer moiety, such that the spacer moiety is cleavable in environments with a lower redox potential, such the cytoplasm and other regions with higher concentrations of molecules with free sulfhydryl groups. Examples of spacer moieties that may be cleaved due to a change in redox potential include those containing disulfides. The cleaving stimulus can be provided upon intracellular uptake of the conjugated protein where the lower redox potential of the cytoplasm facilitates cleavage of the spacer moiety.

In another example, a decrease in pH causes cleavage of the spacer to thereby release of the compound into a target cell. A decrease in pH is implicated in many physiological and pathological processes, such as endosome trafficking, tumor growth, inflammation, and myocardial ischemia. The pH drops from a physiological 7.4 to 5-6 in endosomes or 4-5 in lysosomes. Examples of acid sensitive spacer moieties which may be used to target lysosomes or endosomes of cancer cells, include those with acid-cleavable bonds such as those found in acetals, ketals, orthoesters, hydrazones, trityls, cis-aconityls, or thiocarbamoyls (see for example, U.S. Pat. Nos. 4,569,789, 4,631,190, 5,306,809, and 5,665,358). Other exemplary acid-sensitive spacer moieties comprise dipeptide sequences Phe-Lys and Val-Lys.

Cleavable spacer moieties may be sensitive to biologically supplied cleaving agents that are associated with a particular target cell, for example, lysosomal or tumor-associated enzymes. Examples of linking moieties that can be cleaved enzymatically include, but are not limited to, peptides and esters. Exemplary enzyme cleavable linking moieties include those that are sensitive to tumor-associated proteases such as Cathepsin B or plasmin. Cathepsin B cleavable sites include the dipeptide sequences valine-citrulline, phenylalanine-lysine and/or valine-alanine.

### Protein Complexes

The Fab fragments or derivatives thereof of the invention are preferably conjugated to one or more compounds which render them particularly suitable for use in the RED assay referred to herein. For example, the Fab fragment or derivative thereof may be associated with at least a second polypeptide (referred to hereafter as “the second polypeptide”) to form a protein complex having a molecular size such that the protein complex is retained inside a permeabilised bacterial cell. The Fab fragment or derivative thereof may be associated with the second polypeptide by, for example, covalent bonds such as disulphide bridges, or by non-covalent association. “Non-covalent association” refers to molecular interactions that do not involve an interatomic bond. For example, non-covalent interactions involve ionic bonds, hydrogen bonds, hydrophobic interactions, and van der Waals forces. Non-covalent forces may be used to hold separate polypeptide chains together in proteins or in protein complexes. Thus, the Fab fragment or derivative thereof and second polypeptide may be expressed as separate polypeptides either from the same or different vectors, or one or both of the polypeptides may be expressed from DNA encoding the polypeptides that has been integrated into the bacterial cell genome.

Alternatively, the Fab fragment or derivative thereof and second polypeptide which are associated in a protein complex may be a fusion protein. As used herein, “fusion protein” refers to a hybrid protein, which consists of two or more polypeptides, or fragments thereof, resulting from the expression of a polynucleotide that encodes at least a portion of each of the two polypeptides.

### Protein Complexes Retained in the Permeabilised Bacterial Cell by Molecular Size

The second polypeptide may be any polypeptide having sufficient molecular size, i.e. sufficient molecular weight or molecular radius, such that at least some of the complex formed with the polypeptide being screened for a desired activity is incapable of diffusion from the permeabilised bacterial cell. Thus, the protein complex is retained within the bacterial cell following permeabilisation of the cell. The person skilled in the art will appreciate that the nature of the second polypeptide, including its molecular weight and whether it is a globular or rod (filamentous) protein, will determine its ability to prevent or inhibit diffusion of the protein complex through the bacterial cell wall. In one embodiment, the molecular weight of the second polypeptide is at least about 30 kDa, or at least about 40, 50, 60, 70, 80, 90, 100, 120, 130, 140, 150 or more kDa. In one embodiment, the second polypeptide is at least about 120 kDa.

In one embodiment, the second polypeptide forms multimers having a molecular size greater than the pore-exclusion size of the permeabilised bacterial cell. As used herein, the term “multimer” and grammatical variations thereof refer to formation of a multimeric complex between two or more distinct molecules. The multimer may comprise, for example, two or more molecules of the same protein (i.e. a homo-multimer) or a mixture of two or more different or non-identical proteins (i.e. a hetero-multimer). Proteins that form multimers suitable for use in the methods of the invention include those that form dimers, trimers, tetramers, pentamers, hexamers, and higher order multimers comprising seven or more subunits.

Multimeric proteins include homodimers, for example, PDGF receptor α, and β isoforms, erythropoietin receptor, MPL, and G-CSF receptor, heterodimers whose subunits each have ligand-binding and effector domains, for example, PDGF receptor isoform, and multimers having component subunits with disparate functions, for example, IL-2, IL-3, IL-4, IL-5, IL-6, IL-7, and GM-CSF receptors. Non-limiting examples of other multimeric proteins that may be utilized in the methods of the present invention include factors involved in the synthesis or replication of DNA, such as DNA polymerase proteins involved in the production of mRNA, such as TFIID and TFIIH; cell, nuclear and other membrane-associated proteins, such as hormone and other signal transduction receptors, active transport proteins and ion channels, multimeric proteins in the blood, including hemoglobin, fibrinogen and von Willabrand's Factor; proteins that form structures within the cell, such as actin, myosin, and tubulin and other cytoskeletal proteins; proteins that form structures in the extra cellular environment, such as collagen, elastin and fibronectin; proteins involved in intra- and extra-cellular transport, such as kinesin and dynein, the SNARE family of proteins (soluble NSF attachment protein receptor) and clathrin; proteins that help regulate chromatin structure, such as histones and protamines, Swi3p, Rsc8p and moira; multimeric transcription factors such as Fos, Jun and CBTF (CCAAT box transcription factor); multimeric enzymes such as acetylcholinesterase and alcohol dehydrogenase; chaperone proteins such as GroE, Gro EL (chaperonin 60) and Gro ES (chaperonin 10); anti-toxins, such as snake venom, botulism toxin, Streptococcus super antigens; lysins (enzymes from bacteriophage and viruses); as well as most allosteric proteins. In one embodiment, the multimeric protein is an E. coli protein. Non-limiting examples of E. coli proteins that form multimers include L-rhamnose isomerase (RhnA; for example NCBI accession CAA43002), β-galactosidase (β-gal; for example NCBI accession YP 001461520), betaine aldehyde dehydrogenase (BetB; for example NCBI accession AAA23506), glutamate-5-kinase (G5K; for example NCBI accession AAB08662), glutathione synthase (GshB; for example NCBI accession AP_{—}003504), and a medium chain aldehyde dehydrogenase (YdcW; for example NCBI accession AP_{—}002067).

In one embodiment, the Fab fragment or derivative thereof has a molecular size sufficient to retain the polypeptide within the bacterial cell wall. Thus, the person skilled in the art will appreciate that such a polypeptide need not necessarily be associated with a second polypeptide in order to retain the polypeptide within the permeabilised bacterial cell.

### DNA Binding Proteins

The present inventors have found that DNA is retained within a bacterial cell following permeabilisation. Thus, in one embodiment, the Fab fragment or derivative thereof is associated with a DNA-binding protein to form a protein complex that binds DNA and that is retained inside the bacterial cell. As used herein, “DNA-binding protein” refers to any protein comprising a DNA-binding domain comprising at least one motif that recognizes double-stranded or single-stranded DNA. As would be known to the person skilled in the art, DNA-binding domains include helix-turn-helix, zinc finger, leucine zipper, winged helix, winged helix turn helix, helix-loop-helix, immunoglobulin fold recognizing DNA, or B3 domains. Associating the polypeptide with a DNA-binding protein advantageously provides for enhanced recovery of DNA, for example a plasmid, encoding the polypeptide in the screening methods of the invention.

### Cell Wall Binding Proteins

The Fab fragment or derivative thereof may be associated with a bacterial cell wall-binding protein. The skilled person will understand that the choice of a cell wall-binding protein would depend on the host cell species, as different bacteria have different cell wall compositions. While bacteria have cell walls made up of peptidoglycan (PG), chemical modifications between species can affect cross-species binding. The skilled person will readily be able to determine cell wall-binding proteins suitable for use in a particular bacterial species.

Bacterial cell wall-binding proteins include proteins known to have a domain structure, whereby part of the polypeptide chain in the native structure is able to recognise and bind specific molecules or molecular conformations on the bacterial cell wall. Thus, the term “bacterial cell wall-binding protein” includes a protein domain which is part of the protein which specifically binds to the bacterial cell wall. Examples of bacterial cell wall-binding proteins include the cell wall hydrolases as coded by bacteriophages, cell wall hydrolases of bacteria and different autolysins. Further encompassed are receptor molecules coded by the DNA of bacteriophages and other viruses. Where the bacterial cell wall-binding protein is from hydrolytic enzymes of bacteriophage origin, which are capable of specific binding to bacteria, the cell wall-binding protein maintain their binding ability but preferably have no significant hydrolytic activity.

In one embodiment, the cell wall-binding protein binds non-covalently to the cell wall of E. coli. For example, for an E. coli host cell there are endogenous PG-binding proteins with a conserved ˜100 amino acid PG-binding domain occurring in PAL, OmpA, YiaD, YfiB, and MotB (Parsons et al., 2006). However, proteins from other organisms have been shown to be well expressed in E. coli and to bind the cell wall with high affinity, for example the ˜70 amino acid PG-binding domain from Pseudomonas φKZ phage (KzPG) (Briers et al., 2009). Thus a PG-binding domain from a protein that binds PG may be used as a bacterial cell wall-binding protein in the methods of the invention.

In an exemplary embodiment, the PG-binding domain may be fused to the polypeptide of the invention and expressed in the cytosol of the bacterial cell. Upon membrane permeabilisation, the PG-binding domain binds to the cell wall resulting in the retention of the polypeptide of interest within the permeabilised cell. To potentially further enhance retention of the polypeptide of interest within the cell, the skilled person will understand that the polypeptide may be associated with a DNA-binding protein in addition to a bacterial cell wall-binding protein.

Alternatively, the polypeptide may be associated with a protein that is capable of linking covalently to the bacterial cell wall. Preferably the protein comprises a periplasmic-targeting signal. Thus, the polypeptide is expressed in the cytosol of the bacterial cell, but targeted to the periplasm where it is linked to the cell wall before membrane permeabilisation.

By way of non-limiting example, the bacterial cell wall-binding protein that attaches to the cell wall covalently may be a lipoprotein capable of binding to the cell wall and which lacks a functional N-terminal signal sequence necessary for outer membrane attachment. For example, the lipoprotein may be E. coli LPP. LPP is an abundant E. coli protein that forms a trimeric coiled-coil. In its native form, one end is tethered to the outer membrane via lipidation and the other is covalently bound to the cell wall via a C-terminal lysine. The lipoprotein may further comprise a sequence which targets the lipoprotein to the periplasm, for example an OmpF periplasmic targeting sequence. In one embodiment, the lipoprotein is E. coli lipoprotein lacking a functional N-terminal signal sequence necessary for outer membrane attachment.

In light of the teaching of the present specification, the person skilled in the art will be able to identify or design proteins that attach covalently to the bacterial cell wall and that are suitable for use in the methods of the present invention.

In one embodiment of the invention, the polypeptide of the invention is a fusion polypeptide comprising a KzPG domain and one or more other domains selected from a spacer, SNAP and/or DBP. In one particular embodiment, the fusion polypeptide comprises one or more spacers and the KzPG, SNAP and DBP domains.

### Polynucleotides

The present invention also provides a polynucleotide encoding a Fab fragment or derivative thereof of the invention. Preferably, the polynucleotide is an isolated or recombinant polynucleotide.

The term “isolated polynucleotide” is intended to mean a polynucleotide which has generally been separated from the polynucleotide sequences with which it is associated or linked in its native state. Preferably, the isolated polynucleotide is at least 60% free, more preferably at least 75% free, and more preferably at least 90% free from other components with which it is naturally associated.

The term “recombinant” in the context of a polynucleotide refers to the polynucleotide when present in a cell, or in a cell-free expression system, in an altered amount compared to its native state. In one embodiment, the cell is a cell that does not naturally comprise the polynucleotide. However, the cell may be a cell which comprises a non-endogenous polynucleotide resulting in an altered, preferably increased, amount of production of the encoded polypeptide. A recombinant polynucleotide of the invention includes polynucleotides which have not been separated from other components of the transgenic (recombinant) cell, or cell-free expression system, in which it is present, and polynucleotides produced in such cells or cell-free systems which are subsequently purified away from at least some other components.

“Polynucleotide” refers to an oligonucleotide, a polynucleotide or any fragment thereof. It may be DNA or RNA of genomic or synthetic origin, double-stranded or single-stranded, and combined with carbohydrate, lipids, protein, or other materials to perform a particular activity defined herein. Furthermore, the term “polynucleotide” is used interchangeably herein with the terms “nucleic acid molecule”, “gene” and “mRNA”.

DNA encoding a Fab fragment or derivative thereof can be isolated using standard methods in the art. For example, primers can be designed to anneal to conserved regions within a variable region that flank the region of interest, and those primers can then be used to amplify the intervening nucleic acid, e.g., by PCR. Suitable methods and/or primers are known in the art and/or described, for example, in Borrebaeck (ed), 1995 and/or Froyen et al., (1995). Suitable sources of template DNA for such amplification methods can be derived from, for example, hybridomas, transfectomas and/or cells expressing proteins comprising a variable region, e.g., as described herein.

The polynucleotide of the invention can encode the entire Fab fragment or derivative thereof of the invention. Alternatively, the polynucleotide can encode a single heavy or light chain. Thus, two polynucleotides, each encoding one of the heavy or light chains, can be expressed in a single cell in order to produce the Fab fragment or derivative thereof of the invention.

The polynucleotides of the invention may be mutagenised in order to produce variety in the amino acid sequences of the CDRs and possibly also in the amino acid sequences of the scaffold regions. The person skilled in the art will be aware of suitable methods for this purpose.

The polynucleotide of the invention can also encode a protein conjugate which is or is capable of being conjugated to a Fab fragment or derivative thereof of the invention, as described herein.

### Fab Production

The Fab fragment or derivative thereof disclosed herein can be synthesised by any methods known in the art, such as by the production and recovery of recombinant polypeptides, and by the chemical synthesis of the polypeptides. Thus, the present invention also provides a method of producing the Fab fragments or derivatives thereof of the invention.

The polypeptides (i.e., the Fab fragments or derivatives thereof) of the invention can be produced under reducing, or non-reducing conditions. Preferably, the polypeptides are produced under reducing conditions, such as in the cytoplasm of a host cell.

In the case of a recombinant polypeptide, nucleic acid encoding same is preferably placed into one or more expression vectors, which are then transfected into host cells, for example E. coli cells, yeast cells, insect cells, or mammalian cells, such as simian COS cells, Chinese Hamster Ovary (CHO) cells, or myeloma cells that do not otherwise produce immunoglobulin protein, to obtain the synthesis of proteins in the recombinant host cells. Review articles on recombinant expression in bacteria of DNA encoding the immunoglobulin include Skerra et al., (1993) and Plückthun, (1992). Molecular cloning techniques to achieve these ends are known in the art and described, for example in Ausubel or Sambrook. A wide variety of cloning and in vitro amplification methods are suitable for the construction of recombinant nucleic acids. Methods of producing recombinant immunoglobulins are also known in the art. See U.S. Pat. No. 4,816,567; U.S. Pat. No. 5,225,539, U.S. Pat. No. 6,054,297, U.S. Pat. No. 7,566,771 or U.S. Pat. No. 5,585,089.

Following isolation, the nucleic acid encoding a polypeptide is preferably inserted into an expression construct or replicable vector for further cloning (amplification of the DNA) or for expression in a cell-free system or in cells. Preferably, the nucleic acid is operably linked to a promoter.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Preferred promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Cell free expression systems are also contemplated by the present invention. For example, a nucleic acid encoding a polypeptide can be operably linked to a suitable promoter, e.g., a T7 promoter, and the resulting expression construct exposed to conditions sufficient for transcription and translation. Typical expression vectors for in vitro expression or cell-free expression have been described and include, but are not limited to the TNT T7 and TNT T3 systems (Promega), the pEXP1-DEST and pEXP2-DEST vectors (Invitrogen).

Many vectors for expression in cells are available. The vector components generally include, but are not limited to, one or more of the following: a signal sequence, a sequence encoding protein of the present invention (e.g., derived from the information provided herein), an enhancer element, a promoter, and a transcription termination sequence. The skilled artisan will be aware of suitable sequences for expression of a protein. For example, exemplary signal sequences include prokaryotic secretion signals (e.g., pelB, alkaline phosphatase, penicillinase, Ipp, or heat-stable enterotoxin II), yeast secretion signals (e.g., invertase leader, a factor leader, or acid phosphatase leader) or mammalian secretion signals (e.g., herpes simplex gD signal).

In a preferred embodiment, the polynucleotide encoding the Fab fragment or derivative thereof of the invention is inserted into a vector which is particularly suitable for expression in the RED system described herein. Thus, the vector may be particularly suitable for expression within a bacterial cell. For example, the vector may comprise a site for inserting into the vector a polynucleotide encoding a polypeptide of the invention, and an open reading frame encoding a second polypeptide which associates with the Fab fragment or derivative thereof to form a protein complex that can be retained inside or can attach to the cell wall of a permeabilised bacterial cell. Suitable vectors are described in WO2011/075761.

Preferably, the vector is also capable of replicating within the bacterial cell independently of the host's genome. Suitable vectors include plasmids, viruses and cosmids as well as linear DNA elements, such as the linear phage N15 of E. coli, and/or extrachromosomal DNA that replicates independently of a bacterial cell genome.

The skilled person will be able to readily determine bacterial strains suitable for expressing polypeptides in the methods of the invention. Those skilled in the art would understand that Gram-negative bacteria are suitable for use in the methods of the invention, including, for example, Salmonella, E. coli, Shigella, Campylobacter, Fusobacterium, Bordetella, Pasteurella, Actinobacillus, Haemophilus and Histophilus. In a preferred embodiment, the Gram-negative bacteria is E. coli.

Exemplary promoters that may be included in the vector of the invention include those active in prokaryotes (e.g., phoA promoter, β-lactamase and lactose promoter systems, alkaline phosphatase, a tryptophan (trp) promoter system, and hybrid promoters such as the tac promoter). These promoters are useful for expression in prokaryotes including eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces. Preferably, the host is E. coli. One preferred E. coli cloning host is E. coli 294 (ATCC 31,446), although other strains such as E. coli B, E. coli X 1776 (ATCC 31,537), and E. coli W3110 (ATCC 27,325), DH5a or DH10B are suitable.

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Typical promoters suitable for expression in insect cells include, but are not limited to, the OPEI2 promoter, the insect actin promoter isolated from Bombyx muri, the Drosophila sp. dsh promoter (Marsh et al., 2000) and the inducible metallothionein promoter. Preferred insect cells for expression of recombinant proteins include an insect cell selected from the group comprising, BT1-TN-5B1-4 cells, and Spodoptera frugiperda cells (e.g., sf19 cells, sf21 cells). Suitable insects for the expression of the nucleic acid fragments include but are not limited to Drosophila sp. The use of S. frugiperda is also contemplated.

Means for introducing the isolated nucleic acid molecule or a gene construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD., USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used to produce the Fab fragment or derivative thereof of the invention may be cultured in a variety of media, depending on the cell type used. Commercially available media such as Ham's F10 (Sigma), Minimal Essential Medium ((MEM), (Sigma), RPM1-1640 (Sigma), and Dulbecco's Modified Eagle's Medium ((DMEM), Sigma) are suitable for culturing mammalian cells. Media for culturing other cell types discussed herein are known in the art.

### Scaffold Sequences:

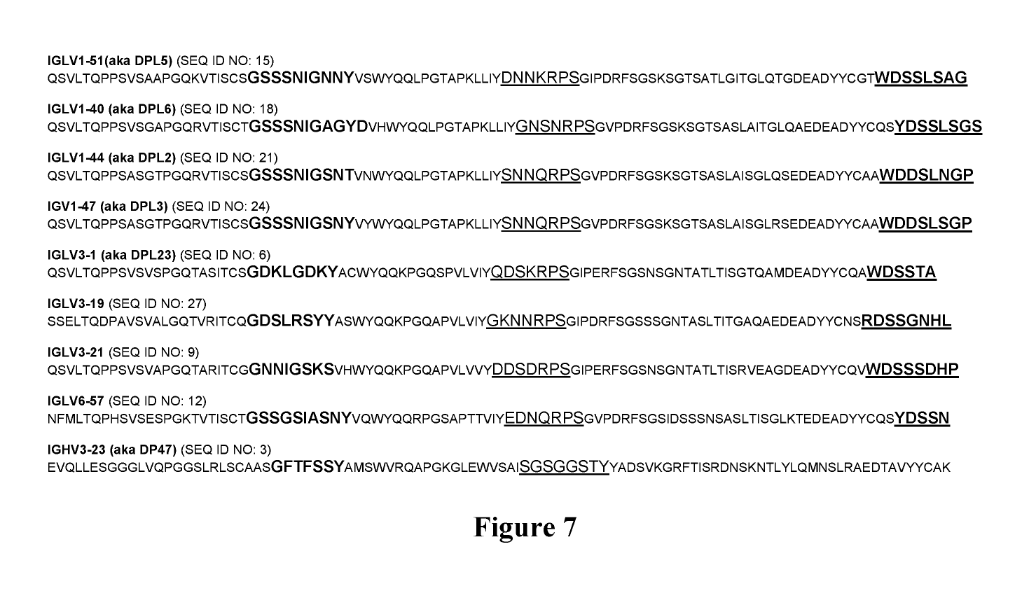

To date, sequences of the structural regions of intrabodies (antibody molecules whose sequences have been engineered or evolved for higher stability such that they may be productively folded in the cytoplasm), i.e. the non-CDR sequences, have differed substantially from the cognate germline genomic sequence. As disclosed herein, the inventors have screened for, and determined, sequences of human antibody variable regions that are identical, or closely related to, the cognate germline genomic sequence, and that allow correct folding and increased stability when expressed in a non-oxidising environment. Preferred sequences for use in the present invention are described below. For any of the variable region sequences described herein, it will be appreciated that the person skilled in the art will be able to identify the CDRs (e.g., many of which are identified on the NCBI database) and the remaining scaffold region. Particular examples of CDRs in each of the variable regions described herein are shown in FIG. 7.

### IGHV3-23

In a preferred embodiment, the polypeptide of the present invention comprises a heavy chain variable region (V_{H}) of the V_{H}3 family of immunoglobulin variable domains. Preferably, the V_{H} is IGHV3-23 (SEQ ID NO: 3).

IGHV3-23, also known as DP-47, belongs to the V_{H}3 family of human Ig variable domains. The V_{H}3 family has 43% (22/51) of the functional members of the V_{H} genes and IGHV3-23 has been cited as the most highly expressed gene in the VH repertoire (Stewart et al., 1993). It is also found at a high frequency in productive Ig rearrangements in B cells (Brezinschek et al., 1997). Because of its high frequency in native Ig repertoires, it has also been frequently isolated from phage display libraries of human V regions (Griffiths et al., 1994). It has also been used as a scaffold partner in synthetic libraries (Jirholt et al., 1998; Pini et al., 1998; Soderlind et al., 2000; Ge et al., 2010). IGHV3-23 was selected as the heavy chain variable region partner in the present inventors' study to identify a stable, soluble antibody variable region scaffold.

Preferably, the polypeptide of the invention comprises an antibody heavy chain variable region (V_{H}) comprising a scaffold region which is at least 90% identical to the scaffold region of IGHV3-23 as set out in SEQ ID NO: 3. For example, the scaffold region may be at least 95%, at least 96%, at least 97%, at least 98%, at least 99%, or 100% identical to the scaffold region of IGHV3-23 as set out in SEQ ID NO: 3. Preferably, the polypeptide of the invention comprises an antibody heavy chain variable region (V_{H}) comprising a scaffold region which is at least 96% identical to the scaffold region of IGHV3-23 as set out in SEQ ID NO: 3. The scaffold region comprises all of the variable region residues excluding the CDR residues. Thus, the polypeptide of the invention may comprise a scaffold region comprising amino acids 1-25, 33-51, 60-98 of SEQ ID NO: 3 (or a scaffold region whose amino acid sequence is at least 90%, at least 95%, at least 96%, at least 97%, at least 98%, at least 99%, or 100% identical to the sequence of these amino acids). Alternatively, the polypeptide of the invention may comprise a scaffold region comprising amino acids 1-25 and 33-98 of SEQ ID NO: 3 (or a scaffold region whose amino acid sequence is at least 90%, at least 95%, at least 96%, at least 97%, at least 98%, at least 99%, or 100% identical to the sequence of these amino acids). In another alternative, the polypeptide of the invention may comprise a scaffold region comprising amino acids 1-51 and 60-98 of SEQ ID NO: 3 (or a scaffold region whose amino acid sequence is at least 90%, at least 95%, at least 96%, at least 97%, at least 98%, at least 99%, or 100% identical to the sequence of these amino acids).

The polypeptides of the invention may comprise any CDR sequence or sequences. Thus, the polypeptides of the invention may comprise the CDR sequences of IGHV3-23 (i.e., amino acids 26-32, 52-59 of SEQ ID NO: 3). Alternatively, the polypeptides of the invention may comprise any other CDR sequence or sequences. Thus, the scaffold region of the V_{H}3 variable domain can serve as a template into which any given CDR sequences can be inserted. The CDR sequences may be randomly generated. Alternatively or in addition, the CDR sequences may be semi-randomly generated (by randomly assigning to each particular amino acid position in the CDR an amino acid residue selected from a subset of all possible amino acids, the subset being known to be necessary or particularly favoured at a given amino acid position in the CDR).

Alternatively, the CDR sequences may be derived from another antibody. Thus, CDRs from e.g. a human antibody can be grafted onto the V_{H}3 variable domain scaffold. It will be appreciated that the person skilled in the art can use various methods to ensure that a scaffold region as defined herein comprises one or more CDR sequences taken from a human antibody. Preferably, such methods include cloning one or more CDR-encoding sequences into a polynucleotide encoding a polypeptide of the invention, as described in more detail herein, below. The CDR-encoding sequences may additionally be varied by targeted or random mutagenesis, in order to provide a plurality of polypeptides comprising a plurality of different CDR sequences. Such methods can be applied to any one or combination of CDR1, CDR2 and CDR3.

Sequences of any one or more of the CDRs CDR1, CDR2 and CDR3 may be introduced into the variable domain scaffolds described herein, in any combination. Preferably, at least the sequence of a CDR3 is introduced into the V_{H}3 variable domain scaffold described herein.

In addition, the length of the CDR sequences introduced into the V_{H}3 variable domain scaffold described herein can be varied. For example, a CDR3 sequence of 15, 14, 13, 12, 11, 10, 9, 8, 7, 6, 5, 4, 3, 2 or 1 amino acids can be inserted into the scaffold.

### IGK and IGL Light Chain Partners to IGHV3-23

The inventors of the present invention, by applying only the criteria that the scFv fusion is soluble in their RED platform (in contrast to previously performed functional screens which required binding of the antibody to an antigen target in vivo and which therefore screened antibodies that were substantially mutated from their respective germline sequences) were able to screen naïve light chains that had not been mutated within the V region. Therefore, they were able to identify germline sequences that conferred solubility upon IGHV3-23 scFv fusions. This has the significant benefit of ensuring that an artificial scaffold library constructed of the V_{L} and V_{H} domains is identical in sequence to abundant human antibody proteins, thereby minimizing immune recognition and rejection on prolonged exposure to any derivatives.

Accordingly, the polypeptide of the invention preferably comprises an antibody light chain variable region (V_{L}) of the V_{L}λ1, 3 or 6 families of immunoglobulin variable domains combined with the human germline IGHV3-23 sequence. Preferred V_{L}λ1, 3 or 6 family members include IGLV1-40 (as set out in SEQ ID NO: 18), IGLV1-44 (as set out in SEQ ID NO: 21), IGLV1-47 (as set out in SEQ ID NO: 24), IGLV1-51 (as set out in SEQ ID NO: 15), IGLV3-1 (as set out in SEQ ID NO: 6), IGLV3-19 (as set out in SEQ ID NO: 27), IGLV3-21 (as set out in SEQ ID NO: 9) and IGLV6-57 (as set out in SEQ ID NO: 12). Thus, the polypeptide of the invention preferably comprises an antibody light chain variable region (V_{L}) comprising a scaffold region which is at least 90% identical to the scaffold region of any of IGLV1-40 (as set out in SEQ ID NO: 18), IGLV1-44 (as set out in SEQ ID NO: 21), IGLV1-47 (as set out in SEQ ID NO: 24), IGLV1-51 (as set out in SEQ ID NO: 15), IGLV3-1 (as set out in SEQ ID NO: 6), IGLV3-19 (as set out in SEQ ID NO: 27), IGLV3-21 (as set out in SEQ ID NO: 9) and IGLV6-57 (as set out in SEQ ID NO: 12). For example, the scaffold region may be at least 95%, at least 96%, at least 97%, at least 98%, at least 99%, or 100% identical to the scaffold region of any one of IGLV1-40 (as set out in SEQ ID NO: 18), IGLV1-44 (as set out in SEQ ID NO: 21), IGLV1-47 (as set out in SEQ ID NO: 24), IGLV1-51 (as set out in SEQ ID NO: 15), IGLV3-1 (as set out in SEQ ID NO: 6), IGLV3-19 (as set out in SEQ ID NO: 27), IGLV3-21 (as set out in SEQ ID NO: 9) and IGLV6-57 (as set out in SEQ ID NO: 12).

Most preferably the V_{L} partner of IGHV3-23 is IGLV3-1 (SEQ ID NO: 6). Thus, the polypeptide of the invention preferably comprises an antibody light chain variable region (V_{L}) comprising a scaffold region which is at least 90% identical to the scaffold region of IGLV3-1 (SEQ ID NO: 6). For example, the scaffold region may be at least 95%, at least 96%, at least 97%, at least 98%, at least 99%, or 100% identical to the scaffold region of IGLV3-1 (SEQ ID NO: 6). The scaffold region of IGLV3-1 may comprise amino acids 1-23, 32-48, 56-89 of SEQ ID NO: 6. Accordingly, the polypeptide of the invention may comprise an antibody light chain variable region (V_{L}) comprising a scaffold region comprising amino acids 1-23, 32-48, 56-89 of SEQ ID NO: 6 (or a scaffold region whose amino acid sequence is at least 90%, at least 95%, at least 96%, at least 97%, at least 98%, at least 99%, or 100% identical to the sequence of these amino acids).

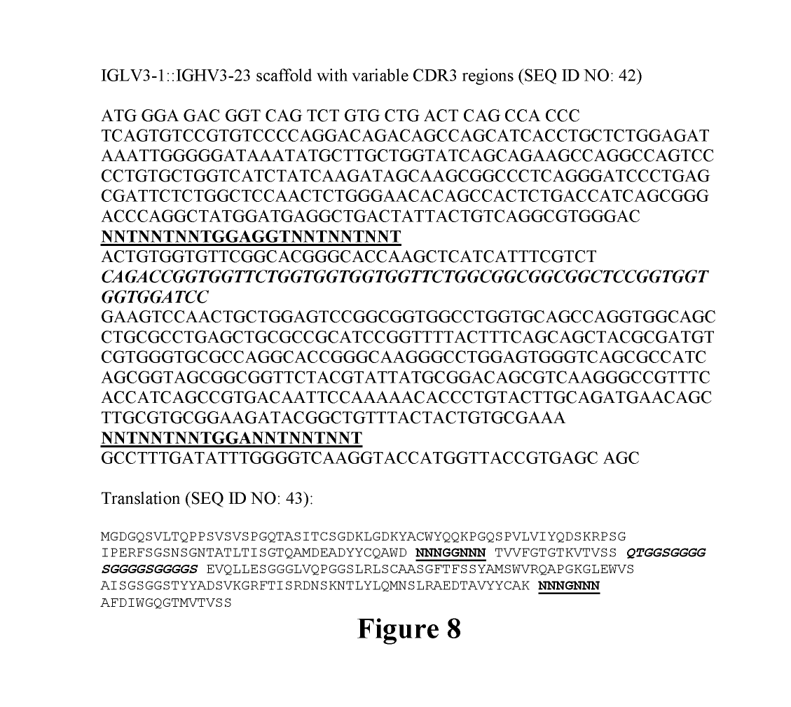

A preferred example of a polynucleotide sequence encoding an IGLV3-1::IGHV3-23 scaffold with variable CDR3 regions, and the corresponding, translated amino acid sequence is illustrated in FIG. 8.

Preferably, the polypeptide of the invention comprises the scaffold region of the V_{L} variable domain (e.g., the polypeptide of the invention may comprise the scaffold region of IGHV3-1, e.g., amino acids 1-23, 32-48, 56-89 of SEQ ID NO: 6). Again, the polypeptides of the invention may comprise any CDR sequence or sequences. Thus, the polypeptides of the invention may comprise the CDR sequences of any of IGLV1-40, IGLV1-44, IGLV1-47, IGLV1-51, IGLV3-1, IGLV3-19, IGLV3-21, and/or IGLV6-57. Alternatively, the polypeptides of the invention may comprise any other CDR sequence or sequences. Thus, the scaffold region of the V_{L} variable domain can serve as a template into which any given CDR sequences can be inserted, as described above in respect of the V_{H}3 variable domain scaffold. Thus, the CDR sequences may be randomly generated. Alternatively or in addition, the CDR sequences may be semi-randomly generated (by randomly assigning an amino acid residue selected from a subset of all possible amino acids to each particular amino acid position in the CDR, the subset being known to be particularly favoured at a given amino acid position in the CDR).

Alternatively, the CDR sequences may be derived from another antibody. Thus, CDRs from e.g. a human antibody can be grafted onto the V_{L} variable domain scaffold. It will be appreciated that various different methods are available to the person skilled in the art to ensure that a scaffold region as defined herein comprises one or more CDR sequences taken from a human antibody. In addition, the CDR sequences of a human antibody may be randomly mutagenised before insertion into the V_{L} variable domain scaffold described herein.

Any one or more of the sequences of CDR1, CDR2 and CDR3 may be inserted into the V_{L} variable domain scaffold described herein, in any combination. Preferably, at least the sequence of a CDR3 is inserted into the V_{L} variable domain scaffold described herein.

In addition, the length of the CDR sequences inserted into the V_{L} variable domain scaffold described herein can be varied. For example, a CDR3 sequence of 15, 14, 13, 12, 11, 10, 9, 8, 7, 6, 5, 4, 3, 2 or 1 amino acids can be inserted into the scaffold.

### Soluble Fab Scaffolds

The V_{L} partners to the V_{H} IGHV3-23 domain that were found to be stable and soluble in the cytoplasm were expressed as a scFv configuration with an 18 aa glycine/serine linker between the variable domains. However, the single polypeptide scFv, although relatively easy to produce, is infrequently used as a therapeutic agent. More commonly, the variable domains are transplanted into a more classic antibody framework structure with conversion into either Fab, F(ab)₂, or a full mAb.

The currently published literature for Fab conversion for scFv sequences is concordant in stating that Fab expression is either into the periplasm of a gram-negative bacterial host such as E. coli, or through the ER/golgi of a eukaryotic host. For example, the Current Protocols chapter “E. coli Expression and Purification of Fab Antibody Fragments” is a good summary of the method for Fab production (Kwong and Radar, 2009) dictating Fab secretion into the E. coli periplasm. Other publications list methods for the production of Fab protein in insect (Johansson et al., 2012) and yeast (Schoonoghe et al., 2012) cells.

The only published method allowing efficient expression of soluble and active Fabs in the cytoplasm of E. coli in the state of the art is expression in strains that have mutations in their thioredoxin reductase (trxB) and glutathione reductase (gor) genes (Levy et al., 2001; Venturi et al., 2002). These mutations enable disulphide bonds to be formed in the cytoplasm, thereby stabilizing the Ig domains of most Fabs, although proteins are most often hemi-disulphide bonded, resulting in mixed populations and lower yields of soluble protein. Furthermore, co-expression of chaperone proteins is also found to be beneficial to yield (Levy et al., 2001; Lobstein et al., 2012). These strains have been marketed commercially as the Origami™ (EMD Millipore) and SHuffle™ (Lobstein et al., 2012; New England Biolabs) strains.

However, although the trxB gor strains may be suitable for expression of single Fab species they have poor DNA transformability and also do not support growth of M13 phage, severely limiting their use for as scaffolds for phage display libraries.

In contrast, the present inventors have achieved particularly effective expression levels of soluble, functional and reduced Fab protein that is produced in the E. coli cytoplasm. This scaffold is exemplary for constructing and screening cytoplasmically-expressed Fab libraries.

The IGLV3-1::IGHV3-23 scFv scaffold that was identified from the cytoplasmic display screen as being one of a number of stable and soluble IGHV3-23 pairings was converted into a bicistronic Fab by replacement of the glycine/serine linker with the downstream IGLC2 domain (SEQ ID NO: 77), followed by a translationally-coupled IGHV3-23 and the CH1 domain from IGHG3 (SEQ ID NO: 78). The DNA and protein sequence of this fusion construct is listed in SEQ ID NO: 79 and SEQ ID NO: 80, respectively. Although the fusion to the IGLC2 and IGHG3 CH1 domains were exemplified, it would be recognized by those skilled in the art that these domains are highly homologous to other genes in the immune repertoire (for example, IGLC2 and IGLC3 are identical at the amino acid level) and could be functionally substituted for other highly homologous members to retain their cytoplasmically soluble character as a full Fab.

In demonstrating that the conversion from scFv to Fab maintained not only its solubility but also its affinity to the target antigen, we transferred the CDR3s of a scFv that bound the fluorescent protein mAG1, from the scFv to the corresponding Fab. This gene was cloned into the cytoplasmic RED display system and was found to show a characteristic soluble appearance microscopically as well as to bind the mAG1 protein (Example 9) Similarly, 3 scFv isolates that bound eGFP were converted to Fab format. These, too, were soluble and bound their fluorescent target (Example 10).

Thus, 4 of 4 scFv to Fab conversions (1 α-mAG1, 3 α-eGFPs) retained their affinity to their antigen target, indicating that the orientation of the V_{L} and V_{H} domains in the IGLV3-1::IGHV3-23 scFv scaffolds were largely unaltered by the addition of the IGLC2 and IGHG3 CH1 domains during conversion to the Fab format.

Thus, we have demonstrated that Fab proteins derived from our IGL3-1::IGHV3-23 scFv scaffold are expressed in soluble form in the cytoplasm, in high yield, and retain the binding affinity of the scFvs to their target antigens.

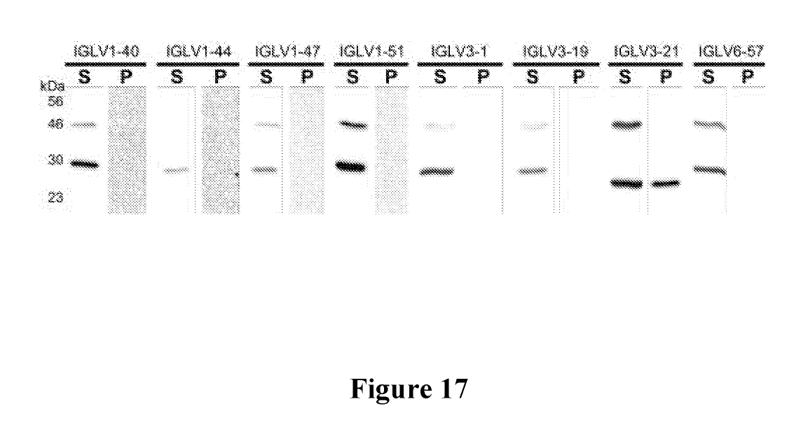

To further extend the exemplification of soluble Fab sequences we converted the remaining germline::V_{H} scFvs identified by the inventors as being cytoplasmically soluble, to wit, pairings of IGVL1-40, IGVL1-44, IGVL1-47, IGVL1-51, IGVL3-19, IGVL3-21 and IGVL6-57 with IGVH3-23, into the Fab format. Example 11 and the corresponding FIG. 17 demonstrate that the conversion successfully produced cytoplasmically soluble Fabs for all pairings.

Although the conversion from scFv to Fab was successfully demonstrated for just those stable germline V_{L}:V_{H} pairings that we identified, it would be recognized by those of skill in the art that this result is likely to be extended to other pairs that are also similarly cytoplasmically stable and soluble.

### Synthetic Fab Libraries

A library of sequences encoding Fab fragments or derivatives thereof may be cloned and expressed in a variety of protein display platforms to select for affinity proteins against a desired target. Thus, the invention provides a library comprising a plurality of Fab fragments or derivatives thereof of the invention. In a preferred embodiment, the libraries may be prepared by identifying a “parent” polypeptide and/or polynucleotide sequence and altering that sequence to create a plurality of variant sequences to form the library. The alteration may be performed by any suitable means, for example, by site directed mutagenesis, or random mutagenesis. Suitable methods of library construction are known in the art.

As indicated above, the variable domains can be cloned directly from a biological source, such that both the structural sequences and the CDRs are present as formed by V(D)J recombination. Alternatively, the Fab library may be partly, or fully, synthetic, with the CDR and the structural regions assembled de novo. For example, a single artificial scaffold representing a pairing of commonly expressed, or particularly stable, antibody genes might be recoded for optimized expression in a host organism. The entire scaffold and CDRs may even be assembled in a single reaction using overlapping oligonucleotides, such as described by Ge et al., (2010).

Methods for building diversity in the antigen-binding CDRs have been fully described by the prior art. They include—sourcing the CDRs from mRNA, from either naïve or pre-immunised immune cells; designing and synthesizing CDRs through analysis of collated antibody sequences; designing and synthesizing CDRs with a weighted amino acid distribution based on collated antibody sequences; adopting a randomized, non-biased CDR region.

Each Ig domain, V_{L} and V_{H}, has three CDR regions, CDR1, CDR2 and CDR3, that are of varying lengths and have different frequencies of interfacial contacts with the antigen. The most variant CDR in vivo for both V_{L} and V_{H} is CDR3, whose loop is formed by recombination between the exon junction of the V-J domains (V_{L}) or the V-D-J domains (V_{H}). This is representative of the naïve immune system. However, once a B-cell has been stimulated for expansion then somatic hypermutation often acts to diversify CDRs 1 and 2 as well.

However, for a cloned Fab library of variable domains built into a single, or a few, V_{L} and V_{H} scaffolds it is common for CDR diversity to be limited to CDR3, with amino acid composition and loop length variation accounting for target binding.

In the instance of the stable polypeptides described by the invention this allows the entirety of the Fab, other than the naturally varying CDR3 loop region, to be identical, or nearly so, to the germline sequence of the cognate antibody genes. This allows screening for affinity proteins that closely resemble the human naïve antibody repertoire, thereby minimizing sequence divergence of an engineered scaffold that might trigger patient immune recognition. Thus, in the present invention, a Fab library may comprise polypeptides differing from one another only in the CDR3 sequence.

An artificial Fab library may be built utilising a single scaffold, or it may be constituted by a plurality of scaffolds. Thus, the library of the present invention may comprise one or more Fab fragments or derivatives thereof having a particular combination of heavy and light chain variable region scaffolds disclosed herein and one or more Fab fragments or derivatives thereof having another, different combination of heavy and light chain variable region scaffolds disclosed herein.

For example, the library may comprise:
one or more Fab fragments or derivatives thereof comprising an antibody heavy chain variable region (V_{H}) comprising a scaffold region which is at least 90% identical to the scaffold region of IGHV3-23 as set out in SEQ ID NO: 3, and an antibody light chain variable region (V_{L}) comprising a scaffold region which is at least 90% identical to the scaffold region of IGLV3-1 (as set out in SEQ ID NO: 6), wherein the V_{H} and the V_{L} are capable of forming an antigen-binding site; and
one or more Fab fragments or derivatives comprising an antibody heavy chain variable region (V_{H}) comprising a scaffold region which is at least 90% identical to the scaffold region of IGHV3-23 as set out in SEQ ID NO: 3, and an antibody light chain variable region (V_{L}) comprising a scaffold region which is at least 90% identical to the scaffold region of any one or more of IGLV1-40 (as set out in SEQ ID NO: 18), IGLV1-44 (as set out in SEQ ID NO: 21), IGLV1-47 (as set out in SEQ ID NO: 24), IGLV1-51 (as set out in SEQ ID NO: 15), IGLV3-19 (as set out in SEQ ID NO: 27), IGLV3-21 (as set out in SEQ ID NO: 9) or IGLV6-57 (as set out in SEQ ID NO: 12), wherein the V_{H} and the V_{L} are capable of forming an antigen-binding site.

Thus, the Fab library of the present invention may comprise one or more Fab fragments or derivatives thereof having any combination of heavy and light chain variable region scaffolds disclosed herein.

A plurality of scaffolds may represent the two broad classes of human Ig genes, namely a heavy chain pairing with the κ and λ lambda light chain classes, or they may be a mixture of a single light chain scaffold with multiple heavy chains, or a mixture of light chain scaffold and a single heavy chain. A plurality of scaffolds could also be drawn from a single member that is the most stable representative of the different subclasses, or could be a combination of only the most stable of scaffolds, belonging to any class.

In the instance of the invention, a Fab library is preferably composed of scaffold regions that are identical, or nearly identical (for example, at least 90, 95, 96, 97, 98 or 99% identical), to the scaffold region of the human IGHV3-23 gene, operably linked to a sequence that is identical, or nearly identical (for example, at least 90, 95, 96, 97, 98 or 99% identical), to the scaffold region of the human genes for IGLV1-40, IGLV1-44, IGLV1-47, IGLV1-51, IGLV3-1, IGLV3-19, IGLV3-21 or IGLV6-53. The library may constitute a single scaffold pairing of a heavy and light chain gene or may be a scaffold pairing of the IGHV3-23 gene with one or more of the aforementioned light chain genes. A library constructed from these scaffolds is demonstrated by the inventors to have superior and desirable stability and solubility properties in the E. coli cytoplasm. In effect, it is a superior intrabody library whose variation from the ideal sequence homology to their cognate germline genes exists only in the CDR loop regions.

It would be recognised by the person skilled in the art that the method of screening for cytoplasmic soluble Fab fragments or derivatives thereof that have VL partners for the VH gene, IGHV3-23, could also be applied for screening for soluble partners of other variable regions, either VL or VH. Furthermore, the method of screening for cytoplasmic soluble Fab fragments or derivatives thereof could be iterated using variants of a single scaffold to find mutations that increase their stability and solubility. For example, any of the scaffold pairs that have been identified (IGHV3-23 with; IGLV1-40, IGLV1-44, IGLV1-47, IGLV1-51, IGLV3-1, IGLV3-19, IGLV3-21 or IGLV6-57) could be used as the template for a further library of variants on a single scaffold with the intention of conducting the cytoplasmic screen at a temperature where the parental scaffold would have poor solubility.

It would also be recognized by experienced practitioners in the art that a Fab library could also be constituted by aforementioned Fab fragments or derivatives thereof present at less than 100% abundance. A library composed of 50% Fab fragments or derivatives thereof described by the invention; or 25% Fab fragments or derivatives thereof described by the invention; or 10% Fab fragments or derivatives thereof described by the invention, would still function to yield affinity proteins of desired stability properties. Thus, the Fab library of the invention may comprise polypeptides other than the Fab fragments or derivatives thereof of the present invention.

Furthermore, although the inventors have surprisingly found that a Fab scaffold that is identical or near identical in sequence to the human germline genes for the V_{H} and V_{L} domains described has superior and desirable stability and solubility properties in the E. coli cytoplasm, it would be recognized by experienced practitioners in the art that these sequences could be obtained to be more polymorphic than reported, yet still function as affinity proteins with desired stability properties. Therefore, the present invention provides a scaffold region with a sequence that diverges from the scaffold region sequences disclosed herein by up to 10%, or 5%, and that still functions to yield affinity proteins with desired solubility and/or stability properties. Thus, the polypeptides of the invention can comprise scaffold region sequences that are at least 90%, 95%, 96%, 97%, 98% or 99% identical to any of the scaffold region sequences disclosed herein.

The present invention provides both a Fab library and a polynucleotide library (for example, a DNA library). DNA libraries are a collection of recombinant vectors containing DNA inserts (DNA fragments) that encode the polypeptide. The origin of the DNA inserts can be genomic, cDNA, synthetic or semi-synthetic.

The cloning and construction of DNA libraries encoding polypeptides of the invention can be performed using methods known in the art. For example, Lutz and Patrick (2004) have reviewed methods of generating library variability and strategies for gene recombination for use in protein engineering. For screening of displayed polypeptide variants, the strategies used for surface-displayed libraries could be adopted and adapted for the methods of the present invention (Becker et al., 2004; Kenrick et al., 2007; Miller et al., 2006; Daugherty et al., 2000).

A library of nucleic acids can be introduced into a plurality of bacterial cells resulting in the expression of a member of the library in each of the bacterial cells. In addition to being expressed, the Fab fragments or derivatives thereof are retained within the permeabilised bacterial cell, or attached to the cell wall, in order to evaluate their function or characteristic. Nucleic acid libraries of a Fab fragment or derivatives thereof, for example, can be generated through a variety of methods including through the introduction of mutations such as point mutations, deletions, and insertions, or through recombination events. Methods for the generation of libraries of variants are known in the art and include error-prone PCR, synthesis of DNA in DNA repair-compromised bacteria, and chemical modification of DNA. Methods for the generation of libraries through recombination are known in the art and include gene shuffling, assembly of DNA in highly recombinogenic bacteria, synthetic nucleic acid library assembly, etc., or any combination thereof. In this way a library of polynucleotides encoding polypeptides can be introduced into a plurality of bacterial cells resulting in the expression of one or members of the library in each of the bacterial cells.

In some embodiments, a library comprises two or more variants of a Fab fragment or derivative thereof wherein each variant comprises a unique polypeptide with a minor change in amino acid sequence, for example, in a CDR sequence. A library can have at least 2, at least 5, at least 10, at least 50, at least 100, at least 1000, at least 10,000, at least 100,000, at least 1,000,000, at least 10⁷ or more members.

### Cytoplasmic Fabs as a Library Scaffold

In one embodiment of the invention, a cytoplasmically-soluble Fab is used as the base scaffold for a diversified library for protein display. Amino acid diversity may be introduced into the variable domain CDR regions, similarly to a scFv library. The assembled Fab protein, with clonal diversity, may be used in cellular display platforms, such as described in WO 2011/075761. Alternatively, it may be linked to the surface of lambdoid phage, which are assembled in the cytoplasm and released on cell lysis, and used in a lambdoid phage display platform, such as described in International Patent Application No. PCT/AU2012/000761. This has been exemplified by Example 12 in which a cytoplasmically soluble Fab is linked to the capsid of a lambda phage via fusion to the vertebrate calmodulin gene. The genotype-phenotype linkage was completed by the co-expression of a lambda capsid head protein, gpD, fusion to an ultra-high affinity calmodulin peptide ligand. Example 12 demonstrates that the Fab display on lambda enabled high capture frequency of the phage (10%) which was >280-fold higher than the background binding.

### Intrabodies and Fab Fragment Derivatives

The cytoplasmically-stable Fab sequences of the invention have utility for use as intrabodies as they may target antigens that are present only in the cytoplasm. They could be delivered intracellularly either by gene delivery (e.g. viral mediated transfection) or as protein transfected using liposomal delivery or by fusion to protein transduction domains (PTDs).

Having a source of low-cost, soluble Fab protein, especially as the output of a rapid in vitro screen, also facilitates the production of bispecific antibodies. A bispecific antibody is one that has the capability to bind two different targets, or target regions, due either to two different Ig binding domains, or one Ig domain and another affinity domain or peptide. Similarly, trispecific, and higher order, binding antibodies are capable of being designed and produced.

In one embodiment, a bispecific antibody is produced to mimic the F(ab)₂ antibodies produced by proteolytic cleavage of the Fc domain.

Two Fabs, each targeting a different antigen, may be linked at their C-term either chemically or by association via peptide domains. By way of non-limiting example, unnatural amino acid incorporation via novel and co-evolved tRNA-aaRS pairs has been demonstrated to incorporate unnatural amino acids into recombinant proteins in E. coli (reviewed by Young and Schultz, 2010). This technology could be used to express two different Fabs, one with a C-terminal amino acid azide, and the other a C-terminal amino acid alkyne. The Fabs could be purified from the cytoplasmic extract and coupled in vitro to form a F(ab)₂ using the cycloaddition ‘click’ chemistry reaction.

In another embodiment of the invention, two cytoplasmically soluble Fabs could be linked at their C-terminus by the association of peptide domains. This association could be stable yet non-covalent, such as formed by the fusion to the two heterodimerising leucine zipper domains from Fos and Jun, for example. To cite another example, this association could be formed by fusion to calmodulin and to a high-affinity calmodulin binding peptide (Montigiani et al., 1996). A stable, covalent linkage could also be formed by the fusion to two peptide domains that associate to form a covalent linkage. For example, fusion of each Fab to one part of the split domain named Spycatcher capable of reassociation and rapid self-ligation (Zakeri et al, 2012).

In yet another embodiment of the invention, the cytoplasmically soluble Fab scaffolds identified could be made into a bispecific antibody by fusion at one C-terminus to a scFv or to a domain antibody. The fusion at the C-terminus could be to either of the light or heavy chain sequences of the Fab. The scFv or single domain antibodies could be of either human origin, or from species known to naturally produce single-domain antibodies such as sharks or camelids, or ‘humanized’ derivatives of the same.

### Screening Methods

The Fab libraries disclosed herein can be used to screen for a fab fragment or derivative thereof that binds to a target molecule. It will be appreciated that Fab fragments or derivatives thereof may be screened for or selected in the context of a library of cells each expressing a different polypeptide or polypeptide variant, or in the context of a single type of cell expressing a single Fab fragment or derivative thereof. The term “target molecule” refers to a molecule that binds to and/or is modified by the Fab fragment or derivative thereof and may be for example an antigen, an enzyme, an antibody, a receptor, etc. Thus, “target molecule” can be used to refer to a substrate such as an enzymatic substrate or a molecule that is being evaluated for binding (e.g., a ligand, eptiope, antigen, multimerization partner such as a homo or hetero dimeric partner, etc., or any combination thereof).

Thus, the invention provides a method of screening for a Fab fragment or derivative thereof that binds to a target polypeptide, the method comprising contacting a Fab fragment or derivative thereof with the target polypeptide, and determining whether the Fab fragment or derivative thereof binds to the target polypeptide. Preferably, a plurality of Fab fragments or derivatives thereof are used in such methods.

A number of suitable screening methods are known in the art, which can be used in accordance with the present invention. For example, the method may comprise a protein display method. The earliest method of protein display is phage display (Smith, 1985), in which the protein of interest is fused to one of the outer-coat proteins of the phage where it may be present along with wild-type copies of the protein. For example, a display platform based on the M13 filamentous phage using fusions to the pIII protein can be used.

Other suitable display methods include ‘in vitro’ display methods where the Fab fragment or derivative thereof is expressed using a cellular translation extract, and the coupling between the Fab fragment or derivative thereof and the coding nucleic acid is achieved through physical linkage (e.g. ribosome display, mRNA display) or through attachment to a common scaffold or encapsulation within a membrane, such as in in vitro compartmentalization (IVC) where the mRNA is translated within a micelle suspension that may also include a microbead (magnetic or sepharose) capture system for both mRNA and protein.

Another suitable method of polypeptide display is microbial surface display which involves the targeted location of expressed polypeptides to the exterior of a microbial cell, either gram-negative, gram-positive eubacteria or yeast. The polypeptides are fused to anchor domains that attach them to the cell surface. The anchor domains may have motifs dictating lipidation or covalent attachment to the cell wall, or they may be a fusion to an integral membrane protein within an exposed loop region.

The present application demonstrates that the Fab fragments and derivatives thereof and polynucleotides of the invention are particularly effective when used in screening methods comprising cell-free expression systems. The use of the Fab fragments or derivatives thereof and polynucleotides of the present invention in such expression systems greatly accelerates the screening process for polypeptides demonstrating high expression, high solubility and high affinity for a target polypeptide. The advantages result from the high solubility, stability and expression demonstrated by the Fab fragments or derivatives thereof, particularly under reducing conditions.

Thus, the Fab fragments or derivative thereofs and polynucleotides of the present invention are particularly suitable for use in a screening method comprising any of the cell-free or in vitro expression systems described herein and others known in the art. For example, Fab fragments or derivative thereofs and polynucleotides of the present invention are particularly suitable for use in a screening method comprising ribosome display, mRNA display, cis-display (wherein an expressed polypeptide remains conjugated to its encoding polynucleotide sequence), or other such methods known in the art.

In addition, the present application demonstrates that the Fab fragments or derivative thereofs and polynucleotides of the invention are particularly effective when used in screening methods based on protein display methods. For example, the Fab fragments or derivative thereofs and polynucleotides of the invention are particularly effective when used in screening methods comprising phage display (e.g., lytic lambda phage, M13 filamentous phage, lysis defective phage, and others known in the art). In one example, the Fab fragments or derivative thereofs and polynucleotides of the invention are surprisingly effective when used in a screening method comprising lambda phage.

In addition, the Fab fragments or derivative thereofs and polynucleotides of the present invention can be used in a screening method based on a lysis defective phage (e.g., as described in International Patent Application No. PCT/AU2012/000761; the content of which is incorporated by reference in its entirety) in combination with the RED system described herein and in WO 2011/075761.

### Kits

The necessary components for performing the methods of the invention may conveniently be provided in the form of a kit. As will be understood to a person skilled in the art, the various components in the kit may be supplied in individual containers or aliquots, or the solution components may be combined in different combinations and at different concentrations to achieve optimal performance of the methods of the invention. It is within the knowledge of the skilled addressee to determine which components of the kit may be combined such that the components are maintained in a stable form prior to use.

In one embodiment, the kit of the invention will at a minimum contain a vector which comprises a site for inserting into the vector a polynucleotide encoding a Fab fragment or derivative thereof of the invention, and an open reading frame encoding a second polypeptide which associates with the first polypeptide to form a protein complex that can be retained inside or can attach to the cell wall of a permeabilised bacterial cell. Preferably, the kit also contains an agent for permeabilising a bacterial cell. In one embodiment, the kit further comprises bacterial cells, preferably Gram-negative bacterial cells. Other additional components may be included with the kit, or other components supplied by the end user, if required.

### Uses

The Fab fragments or derivative thereofs of the present invention are useful in a variety of applications, including research, diagnostic and therapeutic applications. Depending on the antigen to which the polypeptide binds it may be useful for delivering a compound to a cell, e.g., to kill the cell or prevent growth and/or for imaging and/or for in vitro assays. In one example, the Fab fragment or derivative thereof is useful for both imaging and delivering a cytotoxic agent to a cell, i.e., it is conjugated to a detectable label and a cytotoxic agent or a composition comprises a mixture of proteins some of which are conjugated to a cytotoxic agent and some of which are conjugated to a detectable label.

The Fab fragments or derivative thereofs described herein can also act as inhibitors to inhibit (which can be reducing or preventing) (a) binding (e.g., of a ligand, an inhibitor) to a receptor, (b) a receptor signalling function, and/or (c) a stimulatory function. Polypeptides which act as inhibitors of receptor function can block ligand binding directly or indirectly (e.g., by causing a conformational change). The Fab fragments or derivative thereofs described herein may be particularly suitable for applications involving a binding interaction that takes place within a host cell, given the stability and size of preferred polypeptides described herein.

In one embodiment, the invention provides a composition comprising Fab fragment or derivative thereof of the invention. In one particular embodiment, the composition is a pharmaceutical composition comprising a pharmaceutically acceptable carrier.

Compositions for administration will commonly comprise a solution of the Fab fragments or derivative thereofs of the present invention dissolved in a pharmaceutically acceptable carrier, preferably an aqueous carrier. A variety of aqueous carriers can be used, e.g., buffered saline and the like. Other exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like.

The invention will now be further described with reference to the following, non-limiting examples.

### Examples

### Example 1

### Cloning of Human V_{L} Sub-Libraries into a IGHV3-23 Display Vector

To screen for human light chain partners for IGHV3-23 that would be well expressed and soluble in the E. coli cytoplasm, the inventors cloned all 10λ, and 5κ functional light chain families as scFv fusions to IGHV3-23. The scFv library was cloned into an expression construct that was arabinose-inducible and was further fused to downstream domains that conferred cell-wall binding (peptidoglycan (PG) binding domain), an expression reporter domain (SNAP; New England Biolabs), and a DNA binding domain (DBP), in that order. These downstream domains enable retention of the scFv moiety when the outer and inner bacterial host cell membranes are permeabilised by detergent or organic solvents by anchoring the fusion protein to both DNA and the cell wall.

The λ, and κ light chain families were amplified from cDNA prepared from human peripheral blood mononuclear cells (PBMCs). The κ and λ light chain subfamilies were amplified in 7 and 11 PCR reactions respectively. Each sublibrary was first screened separately to broadly characterise the percentage of the library that contained apparently soluble members. Sublibraries that contained an appreciable percentage of soluble members (>1%) were then screened as individual clones.

The oligonucleotide primers were based on the sequences described by Hust and Dubel (2010) with modifications to the ends for cloning via BsmBI. Further changes were made to the reverse primer sequences that were originally designed against the C1 constant domain of the light chain. This was considered to include unnecessary sequence, and degenerate primers were designed against the J regions for the light chain.

The V_{L} genes for λ and κ light chain sublibraries were amplified in two rounds of PCR using Vent DNA polymerase (New England Biolabs). Each sublibrary was cloned separately into the RED display vector using BsmBI (New England Biolabs). Each library was estimated to produce approximately 20-40,000 colonies.

### Example 2

### Screening of Human scFv Fusions Using Retained Encapsulated Display (RED)

As an initial screen of solubility, each library plate was scraped and suspended in 10 mL of LB/glycerol (10%). A fraction of the suspension (˜50 uL) was grown in 1 mL of LB media (10 g tryptone, 5 g yeast extract, 10 g NaCl per litre) at 37° C. for 1 hour and then induced with 0.2% arabinose and grown for a further 2 hours at 25° C. At this point the cells were permeabilised by resuspension of the bacterial pellet in 0.5% n-Octyl-β-D-Thioglucoside (8TGP) in LB media for 10 minutes at 25° C. The permeabilised cells were washed once by pelleting and resuspension in LB media before the induced scFv fusion protein was labeled by the addition of SNAP-surface 488 reagent (S9124S; New England Biolabs) and incubation at 25° C. for 20 minutes. The labeled cells were then washed again by pelleting and resuspended in PBS before a sample was mounted for viewing by fluorescence microscopy.

Microscopic examination showed that although all libraries had some cells within each field of view that appeared to be well expressed and soluble, only the sublibraries representing the Vλ1, Vλ3 and Vλ6 clades were found to have >1% of cells that had a soluble morphology. Thus, the sublibraries for all except for the Vλ1, Vλ3 and Vλ6 were not considered to have a high enough frequency of soluble clones and were not screened further.

Sublibraries Vλ1, Vλ3 and Vλ6 were plated at dilutions that produced single clones and screened for solubility individually. Thus, the Vλ1, Vλ3 and Vλ6 sublibraries were plated at dilutions allowing clean picks of single colonies, which were then induced for expression, and prepared for microscopy, as described above.

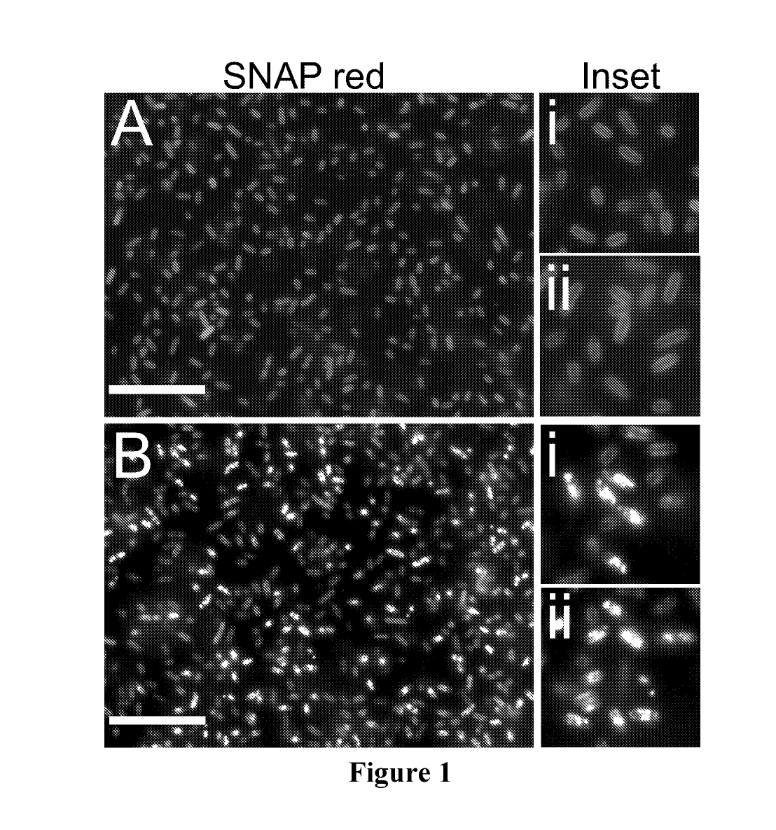

FIG. 1 demonstrates the typical appearance of a well-expressed, soluble scFv clone (1A, and inset), along with a well-expressed, but insoluble scFv clone (1B, and inset). The cells are labelled with SNAP fluorophore following permeabilisation as described above. The distinctive clumping of an insoluble clone constrasts with the more diffuse and peripheral localisation of the soluble clone.

scFv clones that demonstrated soluble expression were then grown overnight at 37° C. under 100 μg/mL ampicillin selection, a plasmid preparation performed using standard methods (Sambrook et al., Molecular Cloning: A Laboratory Manual, 3^{rd} edn, Cold Spring Harbour Laboratory Press (2001) and the DNA was sequenced with a primer in the upstream promoter region of the expression construct. Sequence files were then analysed against the human genomic GenBank database using the NCBI BLAST algorithm.

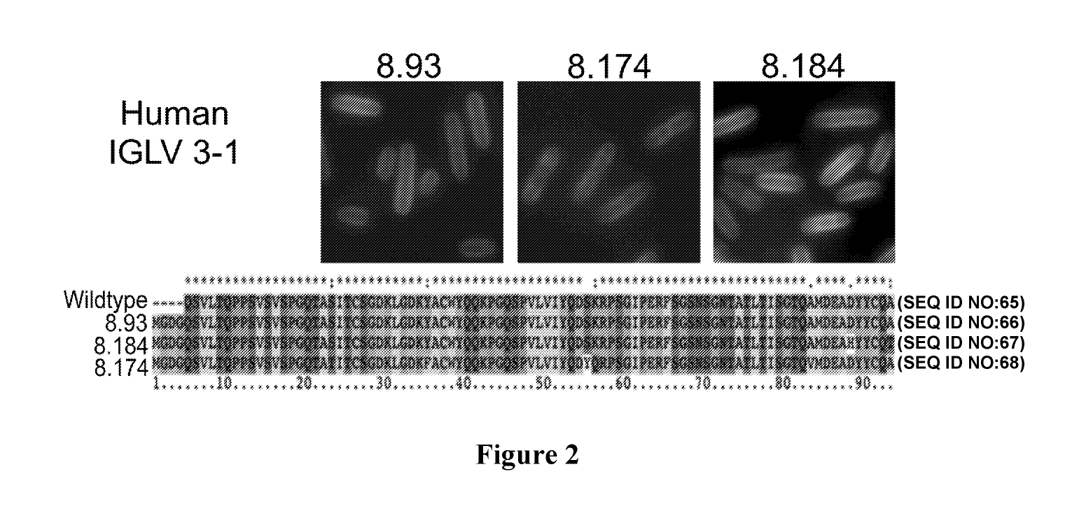

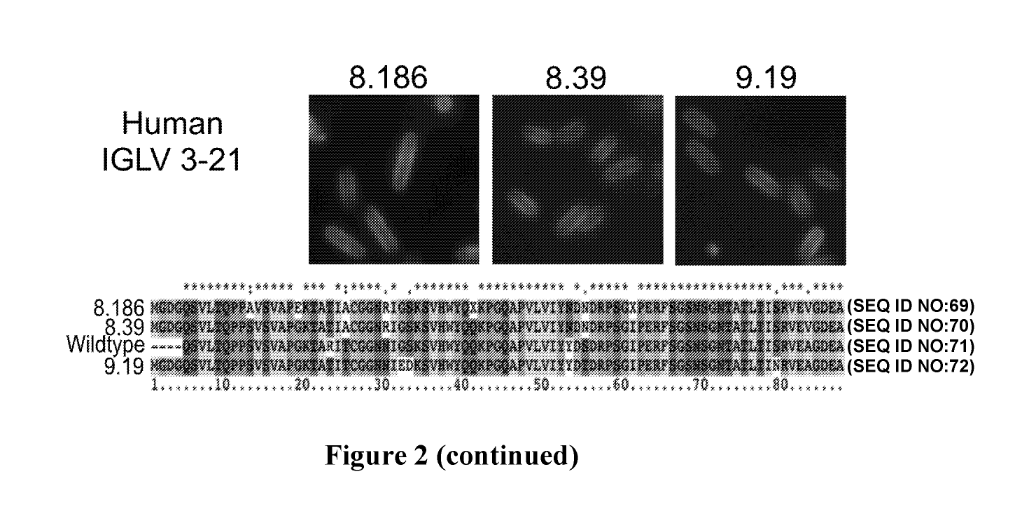

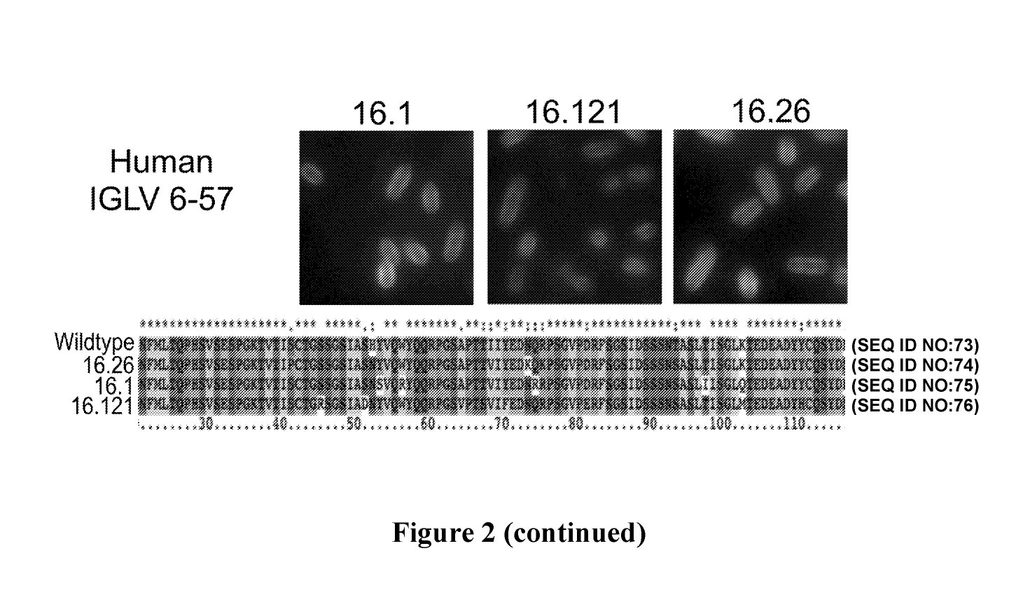

FIG. 2 represents a multiple alignment of selected soluble clones that have high similarity, or total identity, to the V_{L} genes IGLV3-1, IGLV3-21 and IGLV6-57. The multiple alignment was prepared using CLUSTAL X. The images of soluble clone (listed by isolate number) above the alignment correspond to the aligned sequences below.

Clones were checked by isolation and sequencing.

The screen of 779 sublibrary members for soluble individuals yielded 11 clones of IGLV1-40 (SEQ ID NO: 18); 2 clones of IGLV1-44 (SEQ ID NO: 21); 3 clones of IGLV1-47 (SEQ ID NO: 24); 3 clones of IGLV1-51 (SEQ ID NO: 15); 25 clones of IGLV3-1 (SEQ ID NO: 6); 2 clones of IGLV3-19 (SEQ ID NO: 27); 4 clones of IGLV3-21 (SEQ ID NO: 9); 18 clones of IGLV6-57 (SEQ ID NO: 12). Analysis of the sequences of soluble scFv clones showed that there were apparently naïve sequences for members IGLV1-40, IGLV1-51, IGLV3-1, and IGLV3-19 that had not been affinity selected or matured in vivo during B-cell presentation. Furthermore, certain IGLV6-57 clones had high solubility with only 1 (2 clones) or 2 (1 clone) amino acid changes from the translation of the germline sequence, for a total identity of 99% and 98%, respectively.

Therefore, in contrast to the results of prior screening for soluble and stable human antibodies in the cytoplasm of yeast by Tse et al., (2002) who found soluble antibodies that comprised a V_{H}3 domain were entirely paired with VL_{κ} 1 and 4 partners, the inventors found that VL_{κ} subfamilies had poor apparent solubility as a class, with >99% of the VL_{κ} sublibraries clones specifically paired with the IGHV3-23 domain either poorly expressed in E. coli or showing signs of misfolding. Tissot et al. (WO 03/097697) also conducted a Y2H screen for soluble human scFvs, and reported that their soluble scFvs were sequences most closely related to members of the VH1a, VH1b or VH3, clades combined with sequences most closely related to members of the VLκ1 or VLλ1 or VLλ3 clades. However, their optimal configuration was VLλ3 paired with VH1b.

However, as both Tse et al., (2002) and Tissot (WO 03/097697) were applying a functional screen (i.e., binding of the antibody to an antigen target in vivo) as a further requirement for solubility their output antibodies that have a positive Y2H signal required both 1) solubility; and, 2) target binding, and therefore by necessity, were substantially mutated from their germline sequence.

The majority of the V_{L} members isolated in the screen for soluble fusions to the IGHV3-23 domain were IGLV3-1, also known as DPL23. Although some clones had numerous mutations a significant number were identical to the IGLV3-1 germline V sequence (SEQ ID NO: 4), indicating that the germline sequence is inherently stable and soluble in the cytoplasm when partnered with IGHV3-23.

IGLV3-1 has a moderately high expression in the human immune system, representing 15% of the λ light chains (Knappik et al., 2000), but is not the most abundantly expressed λ, member (DPL11). In the published literature it is uncharacterized, lacking any specific citations, and no reported structures with high identity. Although artificial scFv scaffold libraries using IGHV3-23 had been made before, the V_{L} partners were chosen mainly on the basis of their relative expression levels in vivo, i.e. highly expressed DPK22 (Pini et al., 1998; and Ge et al., 2010), DPL3 (aka. IGLV1-47) (Kobayashi et al., 1997; Soderlind et al., 2000) and DPL16 (aka. IGLV3-19) (Viti et al., 2000).

The only published report of a global analysis of the thermostability of the human variable domain repertoire was performed on the Morphosys HuCAL™ library by Ewert et al., (2003). In their article titled “Biophysical Properties of Human Antibody Variable Domains”, the authors examined both the stability of individual domains, as well as the stability of domain pairings (V_{L}::V_{H}), when expressed in the E. coli periplasm.

The V_{H}3 consensus, to which IGHV3-23 is related, was declared the most stable for thermodynamic stability and solubility of the heavy chain variable regions, whilst the Vκ3 consensus was the most stable light chain variable region.

The V_{H}::V_{L} combinations that produced the most stable pairings were those formed between H3::κ3, H1b::κ3, H5::κ3 and H3::κ1. It is noteworthy that none of the most stable pairings included the V_{L}3 family. Furthermore, constructing our scFv library using a V_{H}3 partner (IGHV3-23) was not, in of itself, sufficient to confer stability on the fusion protein when expressed in the E. coli cytoplasm as the vast majority of clones in most sublibraries were either misfolded or poorly expressed.

In summary, on the basis of the prior art, the combination of use of domains IGHV3-23 and IGLV3-1 as a scFv fusion could not be predicted to possess enhanced stability and solubility when expressed in a reducing environment, such as the E. coli cytoplasm.

### Example 3

### Thermostability Testing of scFv Clones

Following the initial screen of the V_{L} sublibraries with induction and expression at 25° C., the scFv clones were subjected to a further screen to grade the clones and families for thermostability.

Each clone was induced at temperatures of 26° C., 28° C., 30° C., 32° C., 34° C., 36° C. and 38° C. for 90 minutes before permeabilising and labelling with SNAP, as described for Example 2. Clones were scored for solubility using fluorescence microscopy, as described for Example 2.

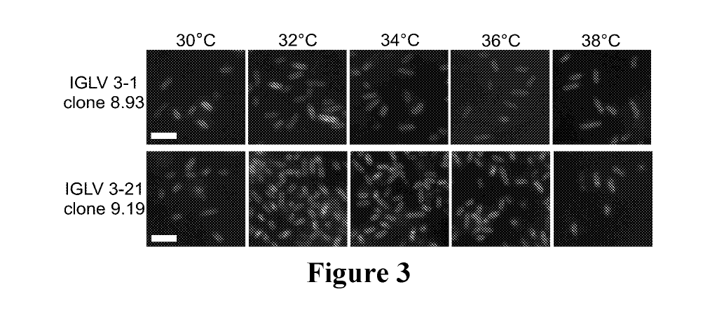

FIG. 3 demonstrates the behaviour of two clones, one IGLV3-1 and one IGLV3-21, with expression at increasing temperatures. The scFv fusion proteins remain soluble until at least 36° C. for the IGLV3-1 clone, although the IGLV3-21 clone shows signs of misfolding between 32 and 34° C.

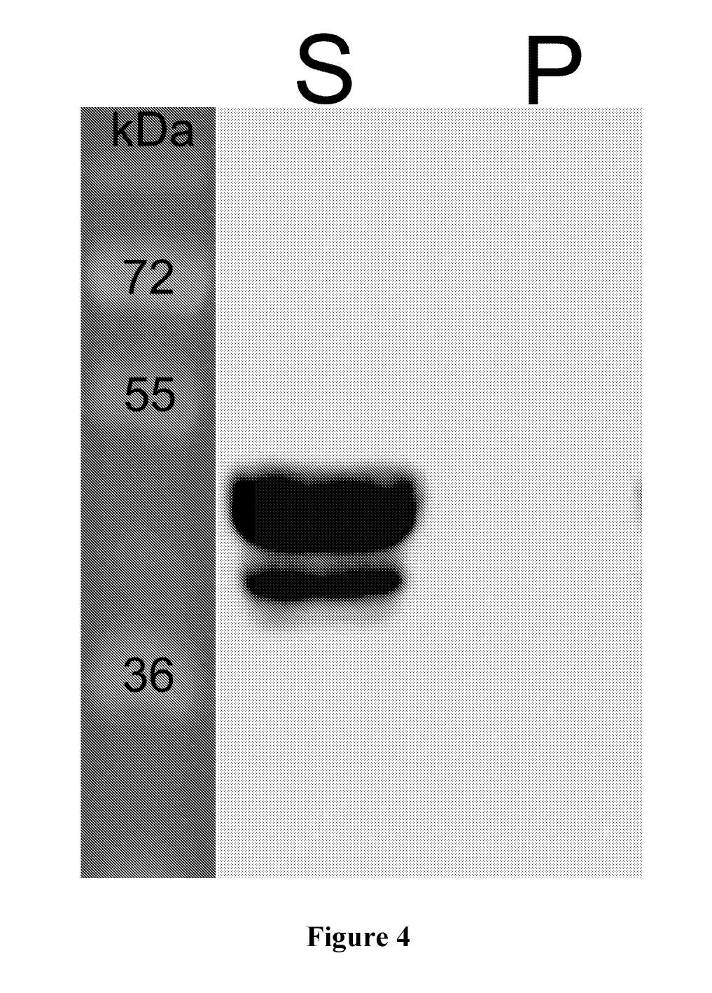

This expression temperature gradient showed that scFv clones related, or identical to, IGLV3-1 and IGLV6-57 were judged as having the best solubility as a class, although individual clones of the other λ, genes also demonstrated varying degrees of solubility. FIG. 4 demonstrates the solubility of representative clones of each species of V_{L} domain isolated from the screen.

That the apparent solubility of the scFvs by microscopy was not artifactual was confirmed by subcloning the scFv fragment, along with the downstream 127 domain from human Titin, into an expression construct with a C-terminal FLAG epitope. The scFv::I27::FLAG fusion protein was induced with arabinose at temperatures ranging from 26° C. to 38° C. and the E. coli cells lysed using ultrasonication. Soluble proteins were separated from insoluble debris and protein aggregates by centrifugation (14K 1 min).

FIG. 4 demonstrates the excellent solubility of an IGLV3-1 clone when expressed in the E. coli cytosol at 25° C. The scFv::I27::FLAG fusion protein is entirely in the soluble fraction. It does demonstrate some N-terminal cleavage of a minor fraction of the total protein, although this was eliminated when the protein was extracted under denaturing conditions suggesting that it was caused by the interaction of periplasmic proteases that were released with the permeabilisation by the 8TGP detergent.

Thus, due to the high frequency of recovery of IGLV3-1 from the solubility screen in E. coli, it was further characterized for the necessary traits for an exemplary scaffold of a soluble scFv library—stability in the cytoplasm at temperatures close to 37° C., and tolerance for a diversified CDR3 loop. The IGLV3-1 was tested for thermostability in the E. coli cytoplasm at a temperature range from 28° C. to 38° C. It was found to be highly soluble to 36° C. when coupled to the light chain J1 and J2 regions, as well as J regions that were formed using the degenerate oligonucleotides as primers during PCR of the PBMC cDNA. At 36° C. and above, it demonstrated a degree of misfolding. This was confirmed by both immunofluorescence and by Western blotting of FLAG-tagged scFv.

### Example 4

### IGLV3-1 J Domain Exchange

The degenerate oligonucleotidea that were used to amplify the VL domains had to prime the 7 different λJ regions in the human genome. As such, the clones isolated from the screen had hybrid λJ regions that represented a non-canonical sequence that may have decreased their folding stability.

TABLE 2
Human λ J regions

| Lambda J | |
|---|---|
| region | Amino acid sequence |
| J1 | VFGTGTKVTVs (SEQ ID NO: 30) |
| J2 | VFGGGTKLTVs (SEQ ID NO: 31) |
| J3 | VFGGGTKLTVs (SEQ ID NO: 32) |
| J4 | VFGGGTQLIIs (SEQ ID NO: 33) |
| J5 | VFGEGTELTVs (SEQ ID NO: 34) |
| J6 | VFGSGTKVTVs (SEQ ID NO: 35) |
| J7 | VFGGGTQLTAs (SEQ ID NO: 36) |

Comparison of the J regions of the most stable of the IGLV3-1 clones that had the germline sequence of the framework regions showed the highest similarity to J regions 1 and 2/3. Therefore, the hybrid J region (“VFGTGTKLIIS” (SEQ ID NO: 37)) was replaced with the germline λJ1 or J2/3 sequences (Table 2) to test whether the thermostability of the IGLV3-1 scaffold would be further improved. The variants were tested at temperatures between 30° C., 32° C., 34° C., 36° C. and 38° C. Subjectively, it was felt that λJ1 gave slightly better folding and solubility than J2/3 or the original hybrid J region of the clone tested.

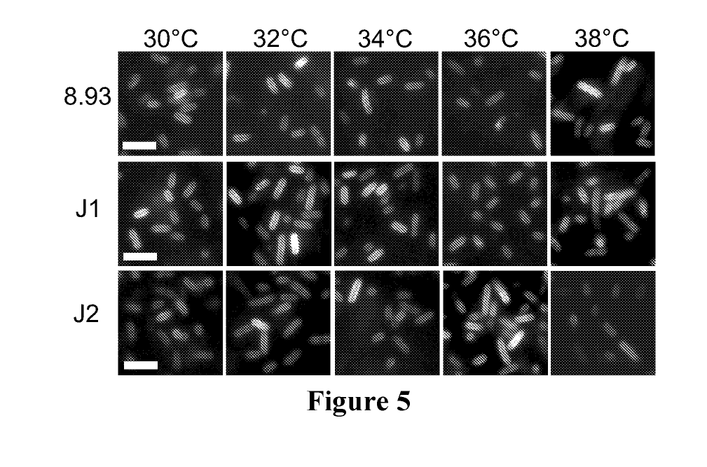

FIG. 5 demonstrates the thermostability behaviour of the original clone (#8.93) with replacement of the λJ region for J1 or J2.

### Example 5

### Tolerance of IGLV3-1 and IGHV3-23 to CDR3 Diversification

For an scFv to be useful as a framework for an affinity library, it needs to be tolerant of substitutions in the CDR3 region. This is especially true for scFvs that are expressed in a reducing environment, such as the E. coli cytosol, where the stabilising intra-domain disulphide bonds are absent.

To test the stability of the preferred scFv scaffold, IGLV3-1::IGHV3-23, the CDR3 region of each domain was diversified separately. Thus, both the IGLV3-1 and IGHV3-23 genes were tested for their tolerance to CDR3 diversification. FIG. 2 shows the region around CDR3 for both sequences, as well as the proposed diversification. The IGLV3-1 CDR3 of 2 amino acids was replaced with a “-NNNGGNNN-” (SEQ ID NO: 28) region (where ‘N’ is an amino acid other than Trp, Gln, Lys, Glu, Met). Similarly, the IGHV3-23 CDR3 of 12 amino acids was replaced with a “-NNNGNNN-” (SEQ ID NO: 29) region. Each domain was tested separately for solubility and expression as a pooled library of clones. In addition, randomly picked individual clones were also tested and sequenced to confirm the expected diversity.

The IGLV3-1 CDR3 was replaced from residue 91 onwards by modifying the IGLV3-1 domain by PCR using the (reverse) oligonucleotide sequence:

(SEQ ID NO: 38)
GATCAGGGTCTGAGACGAGACCGTCACTTTCGTACCGGTGCCGAAC
ACCACAGTANNANNANNTCCGCCANNANNANNGTCCCACGCCTGAC
AGTAATAGTCAGC

The (rev/comp) translation of this sequence gives (where “N” is any amino acid other than Trp, Gln, Lys, Glu, Met):

(SEQ ID NO: 39)
...ADYYCQAWD(91) NNNGGNNN TVVFGTGTKVTVSS

The IGLV3-1 CDR3 replacement was resoundingly successful. The appearance of the population with protein induction at 30° C. was very soluble clones with good expression. 40 clones were analysed individually and 36 were ranked as excellent for solubility and expression. The 4 failed clones, and 16 others with good solubility and expression, were sequenced across the VL domain. It was confirmed that the four failed clones failed due to frameshifts in the long oligonucleotide primer used to amplify up the gene. All other clones examined that had the correct reading frame had a random mixture of amino acids, and demonstrate that the germline IGLV3-1 framework is very tolerant of CDR3 diversification.

Thus, for IGLV3-1, the solubility of a diversified CDR3 library when expressed at 30° C. in the E. coli cytoplasm was surprisingly high. Approximately 90% of clones were soluble with high expression. The 10% clones with low or no expression, or were misfolded, were sequenced and shown to be frameshifted, predominantly in the region of the reverse primer that was by necessity ˜100 bases long. Base deletions are a common error when building synthetic libraries using long oligonucleotides and other groups have developed pre-screening strategies based on antibiotic selection to enrich for in-frame alleles (e.g. Ge et al., 2010).

The CDR3 region of the VH domain, IGHV3-23 was replaced using a reverse oligonucleotide similarly to the method described above from residue 98 onwards by modifying the IGHV3-23 domain by PCR using the (reverse) oligonucleotide sequence:

(SEQ ID NO: 40)
GATCAGGGTCTGAGACCCGCTGCTCACGGTAACCATGGTACCTTGA
CCCCAAATATCAAACGCANNANNANNGCCANNANNANNTTTCGCAC
AGTAGTAAACAGC

The (rev/comp) translation of this sequence gives (where “N” is any amino acid other than Trp, Gln, Lys, Glu, Met):

(SEQ ID NO: 41)
VYYCAK(98) NNNGNNN AFDIWGQGTMVT

The IGHV3-23 proved just as robust to CDR3 diversification as the IGLV3-1 domain, with 80% of tested clones showing soluble, high expression, and the 20% that were poorly expressed were explicable upon sequencing due to conservative mismatches in the framework, or more commonly, single base pair deletions in the region of the long oligonucleotide primer, thereby changing the frame of protein translation.

Thus, for IGHV3-23, the solubility of a diversified CDR3 library when expressed at 30° C. in the E. coli cytoplasm was, again, surprisingly high (˜80%). Again, frameshifting of the fusion protein was responsible for many negatives. The shortening of the CDR3 loop from 12 to 7 amino acids also improved the solubility of this library compared to the parental clone.

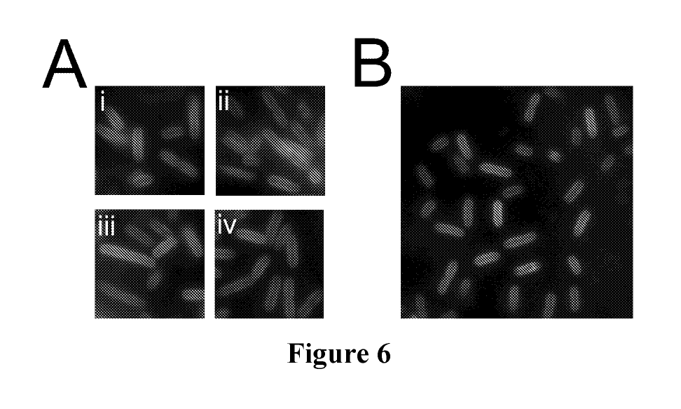

FIG. 6A demonstrates the solubility and high expression of 4 independent clones with the IGLV3-1 CDR3 diversified. FIG. 6B demonstrates a sample of the entire population of clones with the IGHV3-23 CDR3 diversified. Therefore, in summary, the IGLV3-1::IGHV3-23 framework is an exemplary scaffold for constructing an affinity library, being identical to the human germline sequence and remaining robustly soluble with replacement of the CDR3 loops with diversified sequence. Furthermore, combining the scaffold with the RED protein display method in the E. coli cytoplasm enables concurrent screening for both affinity protein stability, expression and binding to the target molecule. The scaffold is highly stable and soluble in the reducing environment of the E. coli cytoplasm where it lacks the stabilizing intra-domain disulphide bonds that are an essential requirement for folding and stability of almost all other scFv proteins. This scaffold will enable low-cost production of affinity reagents in the E. coli cytoplasm for research, therapeutic or diagnostic uses, as well as the use of such reagents in the cytoplasm of mammalian cells for targeting endogenous proteins.

### Example 6

### Construction of a Diversified IGLV3-1::IGHV3-23 scFv Library

The IGLV3-1::IGHV3-23 scaffold was diversified using the strategy described for Example 5 to introduce the amino acid sequences ‘NNNGGNNN’ (SEQ ID NO: 29) and ‘NNNGNNN’ (SEQ ID NO: 30) into the CDR3 regions of VL and VH, respectively.

The diversity was introduced by first creating a base scaffold that consisted of the framework sequence of IGLV3-1 and the J region for IGHV3-23 as follows:

### Framework Sequence:

(SEQ ID NO: 44)
ATG GGA GAC GGT CAG TCT GTG CTG ACT CAG CCA CCC
TCAGTGTCCGTGTCCCCAGGACAGACAGCCAGCATCACCTGCTCTGG
AGATAAATTGGGGGATAAATATGCTTGCTGGTATCAGCAGAAGCCAG
GCCAGTCCCCTGTGCTGGTCATCTATCAAGATAGCAAGCGGCCCTCA
GGGATCCCTGAGCGATTCTCTGGCTCCAACTCTGGGAACACAGCCAC
TCTGACCATCAGCGGGACCCAGGCTATGGATGAGGCTGACTATTACT
GTCAGGCG TGG GAC tgagacctagacggtctct gcg TTT
GAT ATT TGG GGT CAA GGT ACC ATG GTT ACC GTG
AGC AGC TCG TCT CaG ACC.

This framework was cloned into the RED cytoplasmic expression vector with the PG and DNA binding domain elements via flanking BsmBI sites. The intervening sequence (the VL J region and IGHV3-23 framework) (SEQ ID NO: 89) was encoded on a separate plasmid that served as template for a PCR using degenerate primers that contained the CDR3 diversity of both the VL and VH regions at the 5′ and 3′ ends, respectively. These primer sequences had terminal BsaI restriction sites that enabled seamless cloning of the PCR product into appropriately orientated BsaI sites in the scaffold.

### Intervening Sequence:

(SEQ ID NO: 45)
ACT GTG GTG TTC GGC acc ggt acg aaa gtg acT gtc
TCA TCT CAG ACCGGTGGTTCTGGTGGTGGTGGTTCTGGCGGCGG
CGGCTCCGGTGGTGGTGGATCCGAAGTCCAACTGCTGGAGTCCGGCG
GTGGCCTGGTGCAGCCAGGTGGCAGCCTGCGCCTGAGCTGCGCCGCA
TCCGGTTTTACTTTCAGCAGCTACGCGATGTCGTGGGTGCGCCAGGC
ACCGGGCAAGGGCCTGGAGTGGGTCAGCGCCATCAGCGGTAGCGGCG
GTTCTACGTATTATGCGGACAGCGTCAAGGGCCGTTTCACCATCAGC
CGTGACAATTCCAAAAACACCCTGTACTTGCAGATGAACAGCTTGCG
TGCGGAAGATACGGCTGTTTACTACTGTGCGAAA

10 μg of the base scaffold vector was cut with BsaI. The cut vector was precipitated using Sureclean (Bioline). The insert, containing the CDR3 diversity regions, was PCR generated from the core framework as template using primers SEQ ID Nos: 90 and 91. 2 μg of insert PCR was gel-purified before digestion with BsaI. The PCR digest was precipitated using Sureclean. Equimolar amounts of digested vector and PCR insert were ligated using T4 DNA ligase. The ligation was heat-killed and serially electroporated into Argentum E. coli cells (Alchemy Bio). Electroporated cells were spread onto 15 cm LB+carbenicillin (40 μg/mL)+glucose (0.1%) agar plates. The total library size was >1×10⁸ independent clones.

The quality of the library build was assessed by expression of the diversified scFv's. As formerly noted, expression of soluble, partially soluble or insoluble fusion partners can be directly assessed by the appearance of the scFv in the RED display system using the peptidoglycan (PG) binding domain and a chromogenic expression reporter such as SNAP (New England Biolabs). A soluble fusion protein is notably evenly distributed around the perimeter of the cell as it is free to diffuse and bind to the cell wall once the membranes have been permeabilised (e.g. FIG. 1A). In comparison, an insoluble fusion protein forms a densely staining aggregate that does not migrate to the cell wall (e.g. FIG. 1B). A partially soluble fusion has some characteristics of each. We had previously found an excellent correlation between the appearance of a fusion protein, as described above, and the quantity appearing in the soluble/insoluble fractions in Western blots such as the soluble scFv in FIG. 4.

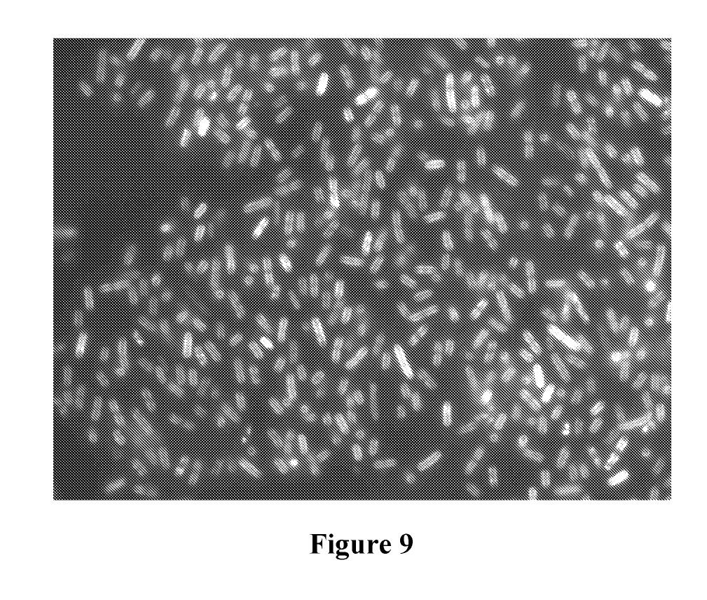

By this empirical standard, our diversified scFv library was composed of ˜90% soluble and well-expressed members, which indicated the tolerance of the IGLV3-1::IGHV3-23 scaffold for the inserted CDR3 diversity. FIG. 9 is a SNAP labeled image of a sample of the expressed library.

To further confirm that the library was composed of randomized V_{L} and V_{H} CDR3 regions, 10 independent clones were sequenced. The sequencing showed that the composition of the CDR3 loops (shown in Table 3) was that expected for a codon diversity created by the ‘NNT’ nucleotide triplet used for degeneracy, i.e. the absence of stop codons, and the amino acids W, Q, M, K, and E.

TABLE 3
Sample CDR3 loop sequences in randomised library

| Clone | VL CDR3 | VH CDR3 |
|---|---|---|
| 1 | PFGGGGYV | PPHGAPA |
| | (SEQ ID NO: 46) | (SEQ ID NO: 47) |
| 2 | LCIGGVAS | HNSGNNF |
| | (SEQ ID NO: 48) | (SEQ ID NO: 49) |
| 3 | FVSGGIST | FNFGNAY |
| | (SEQ ID NO: 50) | (SEQ ID NO: 51) |
| 4 | INSGGASF | XXXGTNY |
| | (SEQ ID NO: 52) | (SEQ ID NO: 53) |
| 5 | SRAGGCNG | FDYGHCI |
| | (SEQ ID NO: 54) | (SEQ ID NO: 55) |
| 6 | TNRGGVCA | TAAGVPF |
| | (SEQ ID NO: 56) | (SEQ ID NO: 57) |
| 7 | Mixed clone | Mixed clone |
| 8 | FSTGGCAF | AICGATA |
| | (SEQ ID NO: 58) | (SEQ ID NO: 59) |
| 9 | FXGGGDGT | PYRGSFF |
| | (SEQ ID NO: 60) | (SEQ ID NO: 61) |
| 10 | IIPGGLYA | PVIGSNT |
| | (SEQ ID NO: 52) | (SEQ ID NO: 63) |

### Example 7

### Screening of the IGVL3-1::IGVH3-23 Library for Binding to mAG1 target

The diversified library was screened for clones that bound to a target protein, mAG. Azami-Green (AG) is a distant ortholog of the Aequorea victoria green fluorescent protein (GFP). Although of low sequence identity (5%), it is similarly green fluorescent with an absorption peak at 492 nm and emission peak at 510 nm. A monomeric form (mAG) was reported by Karasawa et al., (2003) and was re-coded for optimal expression in E. coli by DNA2.0 (USA). A C-terminal E. coli BirA biotinylation motif and 6×His tag was included to aid in purification and mAG matrix attachment.

10¹⁰ cells of the diversified library, representing a ˜100-fold redundancy were induced for RED display as described in Example 2 and in WO 2011/075761. The permeabilised cells were suspended in 50 mL PBS and were labeled with purified mAG that had been pre-bound to MACS streptavidin-conjugated microbeads (130-048-102, Miltenyi Biotec). The cells and microbeads were gently agitated overnight at 4° C. They were then loaded onto 3×LS columns (130-042-401, Miltenyi Biotec) that were fixed to a magnetic support. Each column was washed with 50 mL of PBS. The cells were eluted in 10 mL PBS, pooled and pelleted. Plasmid DNA encoding the library in the RED display vector was isolated from the cell pellet by the alkaline lysis. The plasmid was then electroporated back into Argentum cells and the induction, binding and column purification was repeated. After four iterations of the affinity screen, a low abundance of RED permeabilised cells were observed by fluorescence microscopy to be binding to the mAG protein. At the fifth iteration the permeabilised cells were sorted for mAG binding by FACS. Cells were labeled for FACS using SNAP ligand, to normalise the fusion protein expression, and mAG. FACS of the cell population during the collection of 4,428 mAG-positive events from 2.46×10⁸ total events showed an abundance of approximately 1 binding event in 10⁵ cells. The scFv from the mAG-positive cells that were the FACS output were recovered by PCR using oligonucleotide primers flanking the scFv sequence and the product was re-cloned back into the RED display vector. Analysis of the final screen output for cells that were positive for mAG binding showed that ˜40% (23/60) of the clones were mAG1-positive. Thus, the FACS stage was capable of an ˜10⁵-fold enrichment of positive cells from the library background.

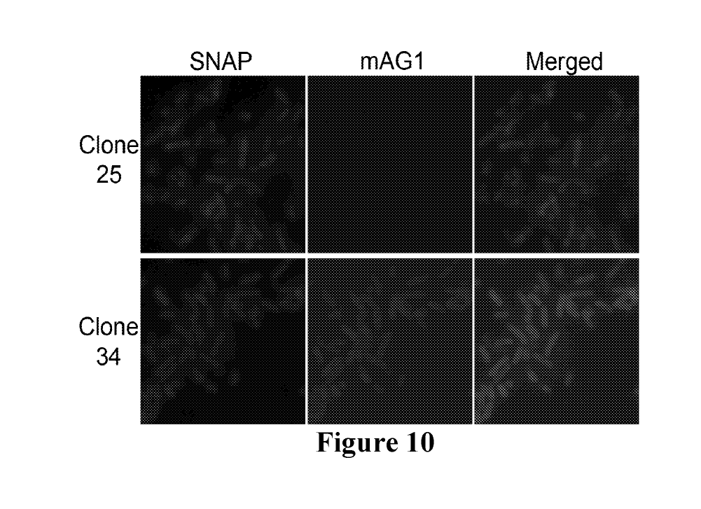

FIG. 10 shows the binding of mAG to RED permeabilised cells for clones that was negative (clone 25) and positive (clone 34) for mAG binding.

20 mAG-positive clones were sequenced and it was found that all 20 were identical. The protein sequence of the mAG-binding IGLV3-1::IGHV3-23 clone is listed in SEQ ID NO: 64 below (with CDR3 sequences in bold and enlarged font, and peptide linker underlined). The VL CDR3 was found to be ‘FNLGGCGD’ and the VH CDR3 ‘HIDGPVA’ which conforms with the designed diversity.

Anti-mAG binding scFv:
(SEQ ID NO: 64)
MGDGQSVLTQPPSVSVSPGQTASITCSGDKLGDKYACWYQQKPGQS
PVLVIYQDSKRPSGIPERFSGSNSGNTATLTISGTQAMDEADYYCQ
AWDFNLGGCGDTVVFGTGTKVTVSSQTGGSGGGGSGGGGSGGGGSE
VQLLESGGGLVQPGGSLRLSCAASGFTFSSYAMSWVRQAPGKGLEW
VSAISGSGGSTYYADSVKGRFTISRDNSKNTLYLQMNSLRAEDTAV
YYCAKHIDGPVAAFDIWGQGTMVTVSSSSQTSILVA

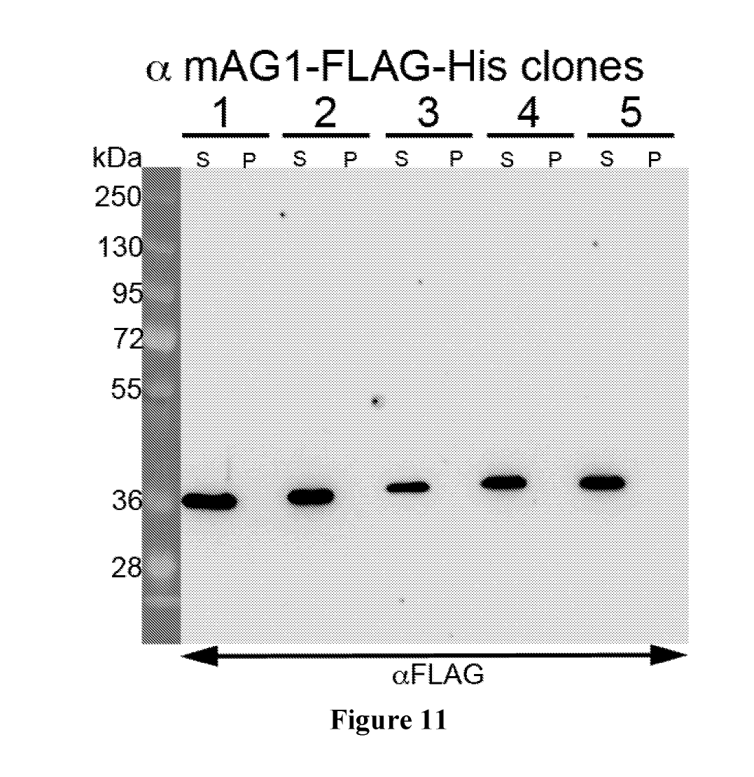

To determine the properties of the α-mAG scFv, the gene was cloned into an expression vector with a C-terminal 6×His and a FLAG epitope tag. scFv expression was induced with arabinose and the cells permeabilised with 0.5% 8TGP to release soluble scFv into the supernatant. The insoluble cellular material was pelleted and samples of both extracts were boiled with SDS-PAGE loading dye and electrophoresed on a 15% SDS-PAGE gel. The resolved proteins were transferred to nitrocellulose membrane and probed with an α-FLAG mouse monoclonal antibody. FIG. 11 demonstrates that the α-mAG scFv was almost exclusively in the soluble fraction.

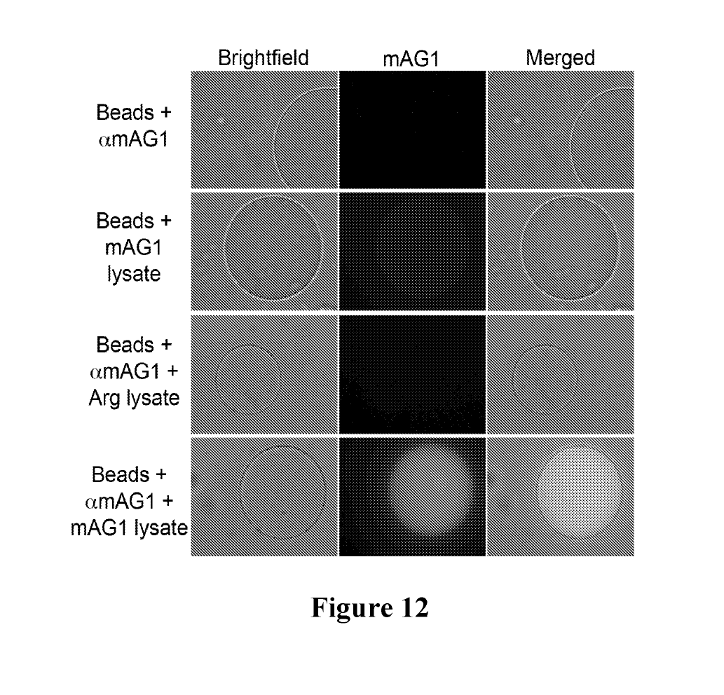

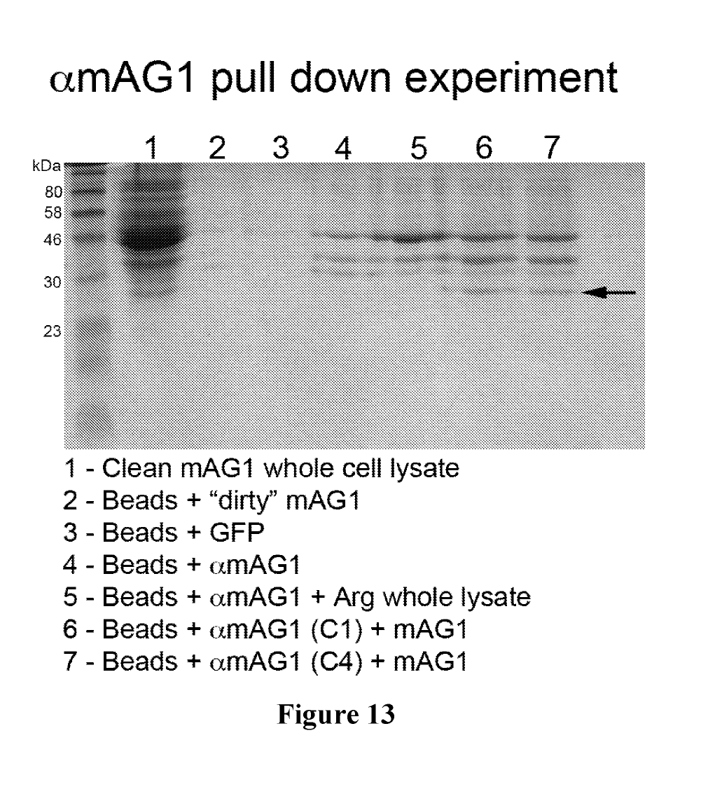

To demonstrate that the α-mAG scFv was specific to mAG protein, and not merely a ‘sticky’ antibody, α-mAG scFv permeabilised cells were labeled with the structurally and functionally mAG related protein, EGFP. These cells, while binding mAG, did not bind EGFP (data not shown). To further evaluate the specificity of the α-mAG scFv, the α-mAG scFv His6-FLAG protein was also bound to IMAC Ni-sepharose resin. A crude cell lysate of ‘clean’ mAG protein (with the His6 and FLAG tags removed) was mixed with the resin. Unbound proteins were washed free. Fluorescence microscopy images in FIG. 12 demonstrate that the resin beads with attached α-mAG scFv bound mAG, whereas control beads did not. The bound proteins were eluted with imidazole and electrophoresed on a SDS-PAGE gel. Coomassie staining of the gel (FIG. 13) demonstrated a band in the α-mAG scFv sample that was of the correct size to be mAG protein with no other bands specific to the mAG cell lysate evident.

Thus, the present invention can be used successfully to generate a library of scFv polypeptides containing randomised CDR3 loops and screened to identify scFvs showing specific binding activity.

### Example 8

### Lambda Phage Display Using the α-mAG IGLV3-1::IGHV3-23 scFv

To demonstrate the utility of a scaffold that exhibits enhanced stability and productive folding in the reducing environment of the cytoplasm, the α-mAG IGLV3-1::IGHV3-23 scFv was cloned as a C-terminal fusion to the lambda bacteriophage gpD capsid protein. Lambda bacteriophage has long been reported as an exemplary vehicle for protein display as it has a number of advantages over filamentous phage. The lambda capsid protein, gpD, is present in ˜400 copies per phage head, and is a robust and tolerant display partner, allowing >80% of the gpD loaded per capsid to be recombinant fusion proteins while maintaining infectious viability (Vaccaro et al., 2006). Furthermore, it is tolerant of fusions to either its N- or C-terminal end. Therefore, a lambda bacteriophage, or equivalently packaged vector, has a multivalent display of the library protein, compared to the nominally single molecule display of filamentous phage. This multivalent display can result in phenomenal capture efficiencies of the phage from a binding solution—up to almost 100% capture (Mikawa et al., 1996). Additionally, the assembly of lambda bacteriophage libraries is facilitated by the commercial availability of kits that enable high efficiency packaging of lambda (up to 2×10⁹ pfu/μg).

However, lambda bacteriophage has not enjoyed the popularity of use of filamentous phage for antibody display due to the singular fact that lambda, and related phage such as P2/P4, P22, T7 and T4, have a lytic lifestyle that results from their assemblage in the cytoplasm. As the great majority of antibody scaffolds are not productively folded without oxidized interdomain disulphide bridges, this has largely precluded the use of lambda bacteriophage for antibody display.

Our identification of a number of IGLV partners for the IGHV3-23 domain that form a cytoplasmically stable scFv scaffold has enabled us to demonstrate the exemplary application of lambda display for antibody screening. The α-mAG scFv was cloned as a C-terminal fusion to the lambda gpD capsid protein with the expression of the fusion protein under control of the arabinose-inducible araBAD promoter and araC regulator. This unit was cloned into the lambda bacteriophage genome similarly to other lambda display platforms (Mikawa et al., 1996; Sternberg and Hoess, 1995; Minenkova et al., 2003), with the notable exception that the lambda genome used was genetically c/857 D⁺ RS⁻. The deletion of the RS genes, which constitute the lambda endolysin (R) and porin (5) genes necessary for cellular lysis, was described in International Patent Application No. PCT/AU2012/000761 for the use of lysis-defective bacteriophage in lambdoid display. A lysis-defective phage vector used for lambdoid display enables the packaging of an infective bacteriophage particle within the cytoplasm. These particles continue to accumulate within the cell, with their capsid fusion protein tethered on their surface at high density, until growth is halted by the researcher processing the host bacterial cells for cytoplasmic RED display. The resultant preparation may thereby be screened for fusion protein antigen binding by FACS. To release the bacteriophage particles that are encapsulated within the permeabilised cell that have been positively sorted by FACS for antigen binding merely requires the addition of a lysozyme. A highly active lysozyme preparation may be purchased commercially for this task (e.g. Ready-Lyse from Epicentre). To complete the recovery of the affinity-selected clones the infectious bacteriophage particles may be infected into host E. coli cells and grown as lysogens. Thus, it should be appreciated by practitioners of the art that the use of lysis-defective phage, in conjunction with a cytoplasmically stable human antibody scaffold, enables high capture frequencies of polyvalent library clones in the free-bacteriophage format, with the final screen being conducted by FACS. Importantly, this change in screening format occurs without any requirement for reformatting of the library expression construct. Thus, this is a screening system that has dual capability for both highly-parallel screening (free bacteriophage panning) with low clonal selectivity and a screen with high clonal selectivity but low throughput (FACS of encapsulated bacteriophage).

To demonstrate the benefits of lambda phage display using the polypeptides of the present invention, the model α-mAG scFv fusion (as one of many suitable examples of the polypeptides of the present invention) was cloned as a C-terminal fusion to the lambda capsid gpD gene.

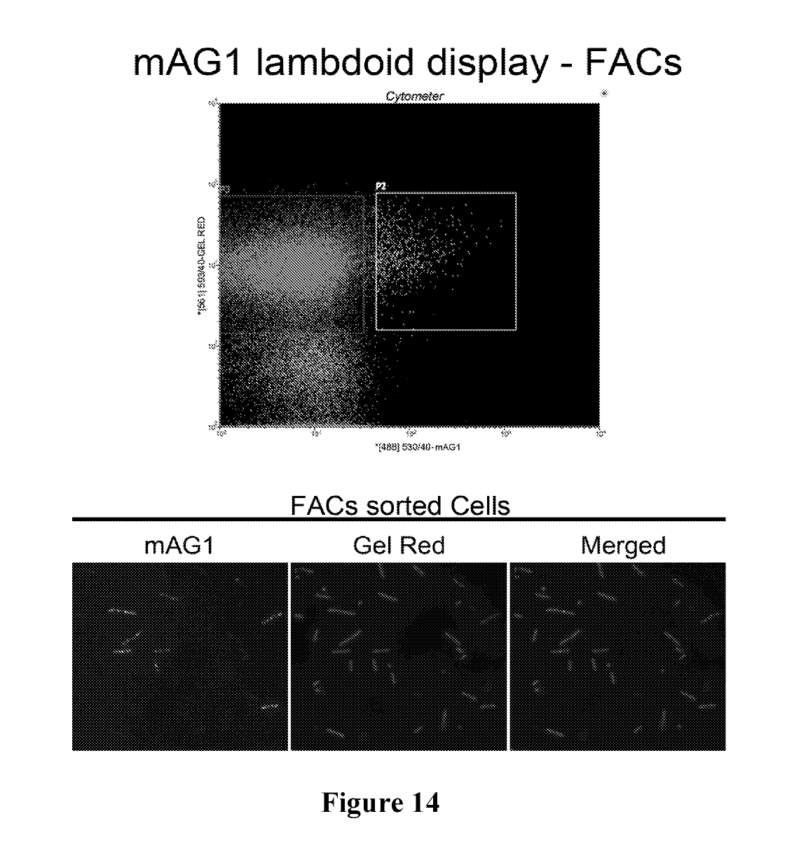

The gpD::α-mAG scFv fusion was then cloned into the lambda display vector under the control of the araBAD promoter. The host cells were induced for lambda packaging by heating the lysogen clone at 42° C. for 15 minutes (the lambda genetic background was cI857 gpD⁺ RS⁻ with the temperature sensitive cI repressor). The fusion protein was induced with 0.2% arabinose immediately following thermal induction. The culture was grown, with aeration, at 32° C. for a further 75 minutes. The cells were pelleted and resuspended in ⅓^{rd} culture volume of LB media+0.5% 8TGP and incubated at 25° C. for 10 minutes to permeabilise the cells by the RED method for screening. To release the phage, 1/10,000^{th} culture volume of Ready-Lyse (Epicentre) lysozyme was added to the suspension. A drop of chloroform was added to inactivate any surviving cells and the bacteriophage particles released were titred for lysogen forming units (cfu/mL). Two bacteriophage stocks were made—one with the construct with the cloned gpD::α-mAG scFv fusion, the other an empty construct. The gpD::α-mAG scFv fusion was diluted to 1 clone in 10⁹ of empty construct, to simulate a starting scFv library density of only a few positive clones. This ‘doped’ library was then panned against biotinylated mAG bound to a streptavidin bead support. The panning was conducted according to methods commonly used for phage panning known to practitioners of the art. Two rounds of panning were conducted with the final round being recovered into the host E. coli strain as lysogens. The third round of screening was conducted by FACS. The lysogen cells were treated as described above for heat-induction of bacteriophage along with arabinose-induction of the gpD::α-mAG scFv fusion. However, instead of releasing the bacteriophage particles with lysozyme treatment, the permeabilised cells were instead incubated with mAG protein. The permeabilised cells were washed once, resuspended in TBS+10 mM MgSO4 and then sorted for mAG binding (i.e. mAG-positive cells would be labeled green) by FACS. FIG. 14 (TOP) shows a screen-grab of the FACS sort in operation demonstrating the incidence of mAG-positive cells. The final incidence of mAG-positive cells post-FACS, assessed by fluorescence microscopy (FIG. 14, BOTTOM), was 20%.

Thus, it has been demonstrated that lambda capsid display, in conjunction with the stable and soluble scFv scaffolds of the present invention, can robustly isolate binding clones from a relatively high starting dilution (1 in 10⁹). Furthermore, when combined with a lysis-defective bacteriophage and treated by the method taught by RED, enables a further magnification of the beneficial properties to include the capability of FACS screening without recloning of the library members.

The combination of these methods greatly accelerate the screening process for antibody clones with ideal properties (high expression, high solubility and high affinity).

### Example 9

### Construction and Expression of an α-mAG1 Fab

The α-mAG1 scFv described in Example 7 was converted into a Fab by linking the V_{L} domain to the IGLC2 (SEQ ID NO: 81) domain to form the light chain protein. Similarly, the V_{H} domain was linked to the IGHG3 CH1 (SEQ ID NO:82) domain to form the heavy chain protein. The heterodimer was expressed as a bicistronic operon by combining an overlapping TGA stop codon of the light chain with an initiation ATG for the heavy chain. The last 10 coding bases of the light chain overlapped with a ribosome binding site for initiation at the heavy chain ATG and also included the FLAG epitope (DYKDDDDK; SEQ ID NO:85) for detection of light chain expression. The downstream heavy chain ORF was frame-shifted from the upstream light chain by two bases. The DNA sequence of the α-mAG1 Fab is listed in SEQ ID NO: 83.

To detect expression of the heterodimeric Fab, and binding to the target antigen, the bicistronic cassette was cloned into the RED cellular display platform. The C-terminus of the Fab heavy chain was fused to a cell-wall (PG) binding domain, SNAP expression reporter and a DNA binding domain (DBP). All elements were under the regulation of the araC promoter. The protein sequence of the α-mAG1 Fab::PG::SNAP::DBP fusion is listed in SEQ ID NO: 84.

It was expected that the protein complex formed, if correctly expressed, would be a Fab heterodimer fused at one end to domains that would tether the Fab to the cell wall of permeabilised E. coli cells. Soluble versus insoluble expression is indicated by the intracellular localization of the SNAP reporter domain, and the co-localisation, in this instance, of the fluorescent reporter protein, mAG1. Soluble protein is found predominantly around the periphery of the cell, bound to the cell wall, whereas insoluble protein forms intracellular aggregates.

The expression of the α-mAG1 Fab::PG::SNAP::DBP fusion protein was induced with arabinose at a temperature of 30° C. for 2 hours. The cells were centrifuged and both inner and outer membranes permeabilised with the detergent 8TGP as described in Example 2. Cells were co-labelled with the SNAP ligand, SNAP surface 549 (New England Biolabs) and mAG1 protein.

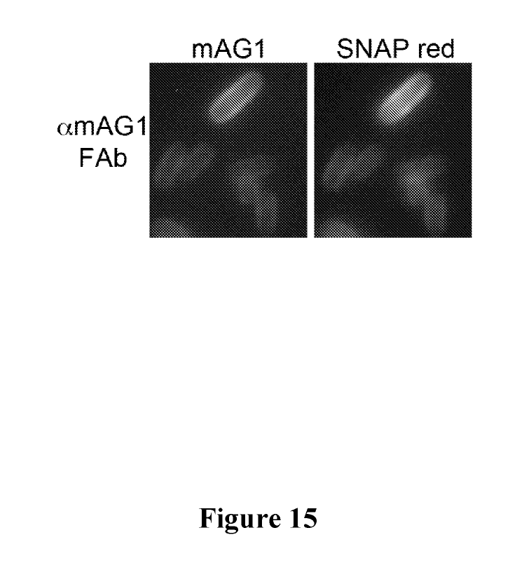

As FIG. 15 demonstrates, the cellular distribution of both the SNAP ligand and mAG1 protein were observed by fluorescence microscopy to be solely around the periphery of the cell, indicating that the Fab heterodimer, and the attached domains, were expressed and highly soluble.

### Example 10

### Construction and Expression of α-eGFP Fabs

An IGLV3-1::IGVH3-23 scFv library of 10¹⁰ independent clones with diversity in both CDR3 regions was constructed as a C-terminal fusion to the lambda bacteriophage gpD coat protein. The library was built using a bacteriophage that was deficient in the R and S endolysin genes for the method of protein display screening using lysis-deficient lambdoid bacteriophage, as described in International Patent Application No. PCT/AU2012/000761.

The library was panned against a protein target, the green fluorescent protein, eGFP. Selected scFv clones that displayed high affinity to eGFP were sequenced. These were converted to a Fab format using the same cloning strategy detailed for the α-mAG1.

Three independent α-eGFP clones with divergent CDR loops were converted from scFv to Fab format to investigate how robust the Fab conversion was for retaining affinity to the target antigen.

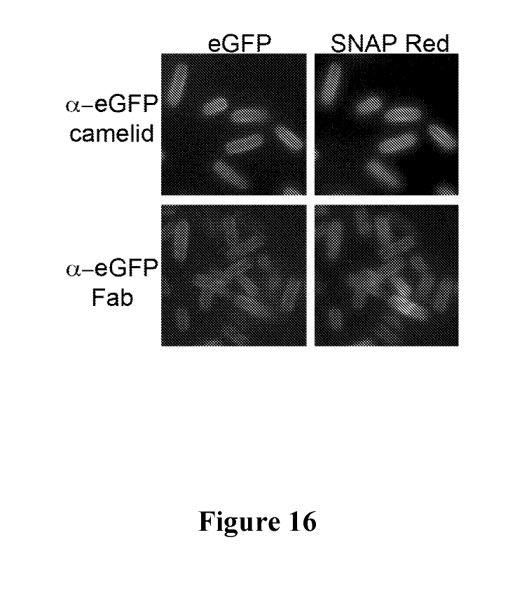

All three clones were expressed as soluble Fab proteins in the RED cell display platform, as evidenced by the cell wall binding of the α-eGFP Fab::PG::SNAP::DBP fusion protein. FIG. 16 demonstrates the binding of eGFP of one such clone. Furthermore, all three clones also retained binding to their eGFP target. Thus, 4 of 4 scFv to Fab conversions (1 α-mAG1, 3 α-eGFPs) retained their affinity to their antigen target, indicating that the orientation of the V_{L} and V_{H} domains in the scFv scaffolds were largely unaltered by the addition of the IGLC2 and IGHG3 CH1 domains during conversion to the Fab format.

### Example 11

### Conversion of Soluble V_{L}/V_{H} Scaffolds to Fab Format and Solubility Testing

The IGLV1-40, IGLV1-44, IGLV1-47, IGLV1-51, IGLV3-1, IGLV3-19, IGLV3-21, and IGLV6-57 V_{L} germline sequences identified as cytoplasmically soluble pairings with the IGHV3-23 germline were fused to a uniform V_{L} CDR3+J region sequence of SSRNTVVFGTGTKVTVSSQT (SEQ ID NO:86). The V_{L} and V_{H} regions were then cloned upstream of the CL and CH1 domains as described in Example 9. The heavy chain gene was further fused to a human calmodulin sequence. Both light and heavy chains were tagged with the FLAG tag epitope (DYKDDDDK; SEQ ID NO:85). The Fab genes were cloned downstream from an arabinose-inducible promoter. An example of the bicistronic Fab::Calmodulin fusion is listed as SEQ ID NO: 87 for the IGLV6-57 fusion.

Expression of the various Fabs in the E. coli cytoplasm was induced by arabinose and the cells were permeabilised with 0.5% 8TGP as described by Example 2. The soluble and insoluble (pellet) fractions were analysed by Western blot using anti-FLAG antibody for detection. FIG. 17 demonstrates that, for 7 of 8 of the Fabs with differing germline VL regions, both light and heavy chains were detected solely in the soluble fraction. For the IGLV3-21::IGLV3-23 pairing the light chain was found predominantly in the soluble fraction, although a significant amount was also insoluble. Both the heavy and light chains of the Fab were found at the expected sizes (˜30 kD for the light chain and ˜45 kD for the heavy chain+calmodulin fusion), although the IGLV3-21 light chain ran slightly smaller than its orthologs.

### Example 12

### Lambda Phage Display of a Soluble Fab

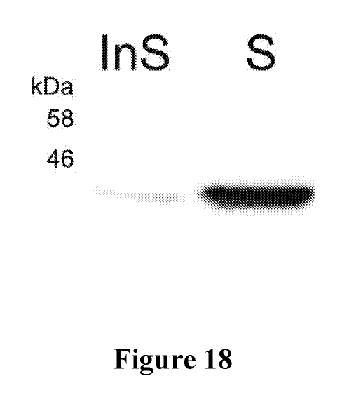

An exemplary use of cytoplasmically-soluble Fab proteins described by the invention would be for the construction of phage display libraries using bacteriophages that are assembled in the cytoplasm. i.e. lytic or lysogenic bacteriophages such as the lambdoid bacteriophages. This utility obviously precludes the use of Fab proteins that are not soluble in the cytoplasm. To demonstrate that the Fab sequences described by the invention could be used in a lambdoid display system the α-mAG1 Fab described in Example 9 was fused to the vertebrate calmodulin gene. Use of calmodulin as a fusion partner for protein association and purification was described in U.S. Pat. No. 6,117,976. A high-affinity peptide (a mutant of the sMLCK M13 peptide) for calmodulin was described by Montigiani et al (1996). With an extraordinary Kd of 2.2×10-12 M and a koff of 2.2×10-6 (s-1), this enables a stable association of the Fab and the lambdoid bacteriophage, which is persistent enough to enable affinity screening. In the lambdoid display example presented, the calmodulin gene was fused directly to the C-terminus of the Fab heavy chain (SEQ ID) whilst the M13 CBP peptide was fused to the C-terminus of the lambda capsid protein, gpD (SEQ ID). Both proteins were temporally co-expressed in the cell during prophage induction as described by Example 8. FIG. 18 demonstrates that the Fab:: calmodulin fusion retains cytoplasmic solubility, with the heavy chain::calmodulin fusion only being found in the soluble protein fraction.

To demonstrate capture of the phage displaying the α-mAG1 Fab, 5.5×107 phage either induced or uninduced (control) for α-mAG1 Fab::calmodulin expression were added to MACS streptavidin beads (130-048-102, Miltenyi Biotec) prebound with biotinylated mAG1 protein. The phage were incubated with MACS beads for 1 hour at 4° C. before being bound, washed and eluted from a MACS M column (130-042-801, Miltenyi Biotec). From the Fab-induced phage, the column output was 5.7×106, a capture efficiency of ˜10%, compared to an output of only 2.0×104 for the uninduced phage. This represents a 280-fold increase in capture efficiency over background.

It will be appreciated by persons skilled in the art that numerous variations and/or modifications may be made to the invention as shown in the specific embodiments without departing from the scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Al-Lazikani et al. (1997) J Mol Biol 273:927-948.
Altschul et al. (1993) J. Mol. Biol. 215: 403410.
Becker et al. (2004) Curr. Opin. Biotech. 15:323-329.
Bork et al. (1994) J. Mol. Biol. 242: 309-320.
Borrebaeck (ed) (1995) Antibody Engineering. Oxford University Press.
Brezinschek et al. (1997) J. Clin. Invest. 99:2488-2501.
Briers et al. (2009) Biochem. Biophys. Res. Comm. 383:187-191.
Chothia and Lesk (1987) J. Mol Biol. 196:901-917.
Chothia et al. (1989) Nature 342:877-883.
Daugherty et al. (2000) J. Immunol. Methods 243:211-227.
Ewert et al. (2003) JMB 325: 531-553.
Froyen et al. (1995) Mol. Immunol. 37: 515-521.
Ge et al. (2010) Biotech Bioeng 106, 347-57.
Griffiths et al. (1994) EMBO J 13:3245-3260.
Guan et al. (1998) Proc. Natl. Acad. Sci. USA, 95: 13206-10.
Higgins and Sharp (1989) CABIOS. 5: 151-153.
Hust and Dubel (2010) Antibody Eng. Chapter 5: Antibody Engineering, Vol 1; Springer.
Jirholt et al. (1998) Gene 215, 471-476.
Johansson et al. (2012) Methods Mol Biol. 907:359-70.
Kabat (1987 and 1991) Sequences of Proteins of Immunological Interest. National Institutes of Health.
Karasawa et al. (2003) JBC 278:34167-34171.
Kenrick et al. (2007) Curr. Prot. Cyt. 4.6.1-4.6.27.
Knappik et al. (2000) JMB 296:57-86.
Kobayashi et al. (1997) Biotechniques 23:500-503.
Kwong and Radar (2009) Curr Protocols Protein Science; Unit 6.10. Wiley Interscience.
Lefranc (2000) Curr. Prot. Imm. 1-37.
Levy et al. (2001) Prot Exp Pur, 23:338-347.
Lobstein et al. (2012) Microb Cell Fact, 11:56.
Lutz and Patrick (2004) Curr. Opin. Biot. 15:291-297.
Marsh et al. (2000) Hum. Mol. Genet. 9:13-25.
Mikawa et al. (1996) JMB, 262:21-30.
Miller et al. (2006) Nat. Meth. 3:561-570.
Minenkova et al. (2003) Int J Can 106:534-544.
Montigiani et al. (1996) JMB 258:6-13.
Parsons et. al. (2006) Biochem. 45:2122-2128.
Pini et al. (1998) JBC 273:21769-21776.
Plückthun (1992) Immunol. Revs., 130:151-188.
Schoonooghe et al. (2012) Methods Mol Bio. 907:325-40.
Skerra et al (1993) Curr. Opinion in Immunol. 5:256-262.
Smith (1985) Science 228:1315-1317.
Soderlind et al. (2000) Nat. Biotech. 18:852-856.
Sternberg and Hoess (1995) PNAS 92:1609-1613.
Stewart et al. (1993) J. Exp. Med. 177:409-418.
Tse et al. (2002) JMB 317:85-94.
Vaccaro et al. (2006) J. Imm. Methods. 310:149-158.
Venturi et al. (2002) JMB 315:1-8.
Vitetta et al. (1993) Immunol. Today 14: 252-259.
Viti et al. (2000) Meth. Enzy. 326:480-505.
Young and Schultz (2010) JBC 285:11039-44.
Zakeri et al. (2012) PNAS 109:E690-7.

1.-47. (canceled)

48. A polynucleotide library comprising a plurality of different polynucleotides, wherein each polynucleotide encodes a Fab fragment or derivative thereof comprising:
a. an antibody heavy chain variable region (VH) comprising a scaffold region which is at least 90% identical to the scaffold region of IGHV3-23 as set out in SEQ ID NO: 3; and
b. an antibody light chain variable region (VL) comprising a scaffold region which is at least 90% identical to the scaffold region of any one of IGLV1-40 (as set out in SEQ ID NO: 18), IGLV1-44 (as set out in SEQ ID NO: 21), IGLV1-47 (as set out in SEQ ID NO: 24), IGLV1-51 (as set out in SEQ ID NO: 15), IGLV3-1 (as set out in SEQ ID NO: 6), IGLV3-19 (as set out in SEQ ID NO: 27), IGLV3-2I (as set out in SEQ ID NO: 9), IGLV6-57 (as set out in SEQ ID NO: 12); wherein the VH and the VL are capable of forming an antigen-binding site,
wherein at least two of the polynucleotides differ from one another by encoding Fab fragments or derivatives thereof comprising one or more different CDRs in the VH and/or VL variable regions.

49. A method of constructing a polynucleotide library, the method comprising preparing a plurality of different polynucleotides encoding a Fab fragment or derivative thereof, which comprises:
a. an antibody heavy chain variable region (VH) comprising a scaffold region which is at least 90% identical to the scaffold region of IGHV3-23 as set out in SEQ ID NO: 3; and
b. an antibody light chain variable region (VL) comprising a scaffold region which is at least 90% identical to the scaffold region of any one of IGLV1-40 (as set out in SEQ ID NO: 18), IGLV 1-44 (as set out in SEQ ID NO: 21), IGLV 1-47 (as set out in SEQ ID NO: 24), IGLV1-51 (as set out in SEQ ID NO: 15), IGLV3-1 (as set out in SEQ ID NO: 6), IGLV3-19 (as set out in SEQ ID NO: 27), IGLV3-2I (as set out in SEQ ID NO: 9), IGLV6-57 (as set out in SEQ ID NO: 12); wherein the V_{H} and the V_{L} are capable of forming an antigen-binding site,
wherein at least two of the polynucleotides differ from one another by encoding polypeptides comprising one or more different CDRs in the V_{H} and/or V_{L} variable regions.

50. An isolated and/or recombinant Fab fragment or derivative thereof comprising:
a. an antibody heavy chain variable region (VH) comprising a scaffold region which is at least 90% identical to the scaffold region of IGHV3-23 as set out in SEQ ID NO: 3; and
b. an antibody light chain variable region (VL) comprising a scaffold region which is at least 90% identical to the scaffold region of any one of IGLV1-40 (as set out in SEQ ID NO: 18), IGLV1-44 (as set out in SEQ ID NO: 21), IGLV1-47 (as set out in SEQ ID NO: 24), IGLV1-51 (as set out in SEQ ID NO: 15), IGLV3-1 (as set out in SEQ ID NO: 6), IGLV3-19 (as set out in SEQ ID NO: 27), IGLV3-2I (as set out in SEQ ID NO: 9), IGLV6-57 (as set out in SEQ ID NO: 12); wherein the V_{H} and the V_{L} are capable of forming an antigen-binding site.

51. The Fab fragment or derivative thereof of claim 50, which is bi-specific or multi-specific.

52. The Fab fragment or derivative thereof of claim 50, which is a fusion polypeptide.

53. The Fab fragment or derivative thereof of claim 50, which is soluble under reducing conditions and capable of stably forming an antigen-binding site when produced under reducing condition.

54. The Fab fragment or derivative thereof of claim 50, which is conjugated to a compound or a linker.

55. A method of screening for a Fab fragment or derivative thereof that binds to a target molecule, the method comprising contacting a Fab fragment or derivative thereof of claim 50 with the target molecule, and determining whether the Fab fragment or derivative thereof of claim 50 binds to the target molecule.

56. The method of claim 55, wherein a polynucleotide encoding the Fab fragment or derivative thereof of claim 50 is expressed in a host cell or in a cell-free expression system to produce the Fab fragment or derivative thereof of claim 50.

57. The method of claim 56, wherein the polynucleotide is expressed in the cytoplasm and/or periplasm of a host cell.

58. The method of claim 57, wherein the host cell is a bacterial cell and the method comprises:
a. culturing a bacterial cell comprising a polynucleotide encoding the Fab fragment or derivative thereof of claim 50 such that the polypeptide is produced,
b. permeabilising the bacterial cell, wherein the polynucleotide and the Fab fragment or derivative thereof is retained inside the permeabilised bacterial cell,
c. contacting the permeabilised bacterial cell with the target molecule such that it diffuses into the permeabilised bacterial cell, and
d. determining whether the Fab fragment or derivative thereof of claim 50 binds to the target molecule.

59. A host cell library comprising a plurality of host cells comprising a Fab fragment or derivative thereof of claim 50, wherein at least one host cell comprises a Fab fragment or derivative thereof that differs from a Fab fragment or derivative thereof present in another host cell in the library in the sequence of amino acids present in one or more CDRs in the V_{H} and/or V_{L} variable domains.

60. The host cell library of claim 59, wherein the Fab fragment or derivative thereof is in the cytoplasm of the host cell.

61. A composition comprising the Fab fragment or derivative thereof of claim 50 and a pharmaceutically acceptable carrier.

62. A non-filamentous phage displaying a Fab or derivative thereof of claim 50 in the cytoplasm of a cell.

63. The non-filamentous phage of claim 62, wherein the phage is a lambdoid phage.

64. The non-filamentous phage of claim 62, wherein the phage is a lysis-defective phage.

65. A method for screening a polynucleotide library for nucleotide sequences encoding a Fab fragment or derivative thereof of claim 50 that bind a target molecule, the method comprising:
a. transforming a host cell with a polynucleotide encoding the Fab fragment or derivative thereof of claim 50,
b. cultivating the transformed host cell under conditions suitable for expression and assembly of a non-filamentous phage particle comprising a coat protein fused or linked to the heavy chain variable region and/or antibody light chain variable region of the Fab fragment or derivative thereof, and
c. determining whether the Fab fragment or derivative thereof of claim 50 binds to the target molecule.
